SEQUENCE LISTING (8 sequences)

SEQ ID NO: 1 - DNA, 5617 bp, Homo sapiens
agtcccgcgaccgaagcagggcgcgcagcagcgctgagtgccccggaacgtgcgtcgcgcccccagtgtccgtcgcgtccgccgcgccccgggcggggatggggcggccagactgagcgccgcacccgccatccagacccgccggccctagccgcagtccctccagccgtggccccagcgcgcacgggcgatggcgaaggcgacgtccggtgccgcggggctgcgtctgctgttgctgctgctgctgccgctgctaggcaaagtggcattgggcctctacttctcgagggatgcttactgggagaagctgtatgtggaccaggcagccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggcatgtcagacccgaactggcctggagagagtcctgtaccactcacgagagctgatggcactaacactgggtttccaagatatccaaatgatagtgtatatgctaactggatgctttcaccctcagcggcaaaattaatggacacgtttgatagttaacatttctttgtgaaaggtaatggactcacaaggggaagaaacatgctgagaatggaaagtctaccggccctttctttgtgaacgtcacattggccgagccgtgttcagttcccaggtggcagactcgtttttggtagtttgttttaacttccaaggtggttttacttctgatagccggtgattttccctcctagcagacatgccacaccgggtaagagctctgagtcttagtggttaagcattcctttctcttcagtgcccagcagcacccagtgttggtctgtgtccatcagtgaccaccaacattctgtgttcacatgtgtgggtccaacacttactacctggtgtatgaaattggacctgaactgttggatttttctagttgccgccaaacaaggcaaaaaaatttaaacatgaagcacacacacaaaaaaggcagtaggaaaaatgctggccctgatgacctgtccttattcagaatgagagactgcggggggggcctgggggtagtgtcaatgcccctccagggctggaggggaagaggggccccgaggatgggcctgggctcagcattcgagatcttgagaatgattttttttaaatcatgcaacctttccttaggaagacatttggttttcatcatgattaagatgattcctagatttagcacaatggagagattccatgccatctttactatgtggatggtggtatcagggaagagggctcacaagacacatttgtcccccgggcccaccacatcatcctcacgtgttcggtactgagcagccactacccctgatgagaacagtatgaagaaagggggctgttggagtcccagaattgctgacagcagaggctttgctgctgtgaatcccacctgccaccagcctgcagcacaccccacagccaagtagaggcgaaagcagtggctcatcctacctgttaggagcaggtagggcttgtactcactttaatttgaatcttatcaacttactcataaagggacaggctagctagctgtgttagaagtagcaatgacaatgaccaaggactgctacacctctgattacaattctgatgtgaaaaagatggtgtttggctcttatagagcctgtgtgaaaggcccatggatcagctcttcctgtgtttgtaatttaatgctgctacaagatgtttctgtttcttagattctgaccatgactcataagcttcttgtcattcttcattgcttgtttgtggtcacagatgcacaacactcctccagtcttgtgggggcagcttttgggaagtctcagcagctcttctggctgtgttgtcagcactgtaacttcgcagaaaagagtcggattaccaaaacactgcctgctcttcagacttaaagcactgataggacttaaaatagtctcattcaaatactgtattttatataggcatttcacaaaaacagcaaaattgtggcattttgtgaggccaaggcttggatgcgtgtgtaatagagccttgtggtgtgtgcgcacacacccagagggagagtttgaaaaatgcttattggacacgtaacctggctctaatttgggctgtttttcagatacactgtgataagttcttttacaaatatctatagacatggtaaacttttggttttcagatatgcttaatgatagtcttactaaatgcagaaataagaataaactttctcaaattattaaaaatgcctacacagtaagtgtgaattgctgcaacaggtttgttctcaggagggtaagaactccaggtctaaacagctgacccagtgatggggaatttatccttgaccaatttatccttgaccaataacctaattgtctattcctgagttataaaagtccccatccttattagctctactggaattttcatacacgtaaatgcagaagttactaagtattaagtattactgagtattaagtagtaatctgtcagttattaaaatttgtaaaatctatttatgaaaggtcattaaaccagatcatgttcctttttttgtaatcaaggtgactaagaaaatcagttgtgtaaataaaatcatgtatcataaaa

SEQ ID NO: 2 - protein, 1114 aa, Homo sapiens
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGMSDPNWPGESPVPLTRADGTNTGFPRYPNDSVYANWMLSPSAAKLMDTFDS

SEQ ID NO: 3 - protein, 1114 aa, Artificial Sequence
  Exemplary RET protein with V804X mutation
  MISC_FEATURE (804)..(804): X is any amino acid other than valine or glutamic acid
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIXEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGMSDPNWPGESPVPLTRADGTNTGFPRYPNDSVYANWMLSPSAAKLMDTFDS

SEQ ID NO: 4 - protein, 1114 aa, Artificial Sequence
  Exemplary RET protein with Y806X mutation
  MISC_FEATURE (806)..(806): X is any amino acid other than tyrosine
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEXAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGMSDPNWPGESPVPLTRADGTNTGFPRYPNDSVYANWMLSPSAAKLMDTFDS

SEQ ID NO: 5 - protein, 1114 aa, Artificial Sequence
  Exemplary RET protein with A807X mutation
  MISC_FEATURE (807)..(807): X is any amino acid other than alanine
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYXKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGMSDPNWPGESPVPLTRADGTNTGFPRYPNDSVYANWMLSPSAAKLMDTFDS

SEQ ID NO: 6 - protein, 1114 aa, Artificial Sequence
  Exemplary RET protein with K808X mutation
  MISC_FEATURE (808)..(808): X is any amino acid other than lysine
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAXYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGMSDPNWPGESPVPLTRADGTNTGFPRYPNDSVYANWMLSPSAAKLMDTFDS

SEQ ID NO: 7 - protein, 1114 aa, Artificial Sequence; identical to SEQ ID NO: 2
  Exemplary RET protein with Y809X mutation
  MISC_FEATURE (809)..(809): X is any amino acid other than tyrosine

SEQ ID NO: 8 - protein, 1114 aa, Artificial Sequence
  Exemplary RET protein with G810X mutation
  MISC_FEATURE (810)..(810): X is any amino acid other than glycine
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYXSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGMSDPNWPGESPVPLTRADGTNTGFPRYPNDSVYANWMLSPSAAKLMDTFDS

(19) United States
(12) Patent Application Publication
(10) Pub. No.: US 2024/0000791 A1
(43) Pub. Date: Jan. 4, 2024

(54) Methods of Using 4-Amino-N-[4-(Methoxymethyl)Phenyl]-7-(1-Methylcyclopropyl)-6-(3-Morpholinoprop-1-YN-1-YL)-7H-Pyrrolo[2,3-D]Pyrimidine-5-Carboxamide for the Treatment of Tumors

(71) Applicants: Helsinn Healthcare SA, Lugano/Pazzallo (CH); Taiho Pharmaceutical Co., Ltd., Tokyo (JP)

(72) Inventors: Emanuela Lovati, Mendrisio (CH); Ruben Giorgino, Allschwil (CH); Simona Doria, Monza (IT); Claudio Giuliano, Como (IT); Annalisa Bonifacio, Sorengo (CH); Miharu Igarashi, Tokyo (JP); Isao Miyazaki, Ibaraki (JP); Masanori Kato, Ibaraki (JP); Alberto Bernareggi, Concorezzo (IT)

(73) Assignees: Helsinn Healthcare SA, Lugano / Pazzallo (CH); Taiho Pharmaceutical Co., Ltd., Tokyo (JP)

(21) Appl. No.: 18/253,229

(22) PCT Filed: Nov. 18, 2021
(86) PCT No.: PCT/EP2021/082120

§ 371 Date: May 17, 2023

Related U.S. Application Data
(60) Provisional application No. 63/116,282, filed on Nov. 20, 2020.
(60) Provisional application No. 63/229,626, filed on Aug. 5, 2021.

(51) Int. Cl. A61K 31/5377; A61P 35/00

(52) U.S. Cl. CPC A61K 31/5377; A61P 35/00

(57) ABSTRACT

The present invention relates to compositions and methods for treating patients with cancer having a RET gene abnormality comprising administering HM06/TAS0953, for example patients with non-small cell lung cancer (NSCLC), and that may also have brain and/or leptomeningeal metastases, or another solid tumor; where the patient is administered an effective amount of HM06/TAS0953, where the HM06/TAS0953 can be formulated in a composition and administered orally in a single or multiple doses; and where the patients may have previously received and/or have developed resistance to another RET-selective or multi-kinase inhibitor.

### I. CROSS-REFERENCE TO RELATED APPLICATIONS

This application claims the benefit of priority of U.S. Provisional Application No. 63/116,282, filed Nov. 20, 2020; and U.S. Provisional Application No. 63/229,626, filed Aug. 5, 2021; each of which is incorporated by reference herein in its entirety for any purpose.

### II. FIELD

This application relates to compositions and methods for treating patients with cancer having a RET gene abnormality comprising administering HM06.

### III. BACKGROUND

Receptor tyrosine kinases (RTKs) play an important role in a variety of cellular processes including growth, motility, differentiation, and metabolism. As such, the dysregulation of RTK signaling leads to an assortment of human diseases, such as cancers. There are various alterations in the genes encoding RTKs, such as EGFR, HER2/ErbB2, MET and RET (REarranged during Transfection). RET is a single-pass transmembrane receptor tyrosine kinase that is required for normal development, maturation, and maintenance of several tissues and cell types. (Airaksinen M S et al., Nat Rev Neurosci. 2002, 3(5):383-394; Alberti L et al., J Cell Physiol. 2003, 195(2):168-186.) Activation of RET occurs via oncogenic mutations in familial and sporadic cancers—for example, those of the thyroid (papillary/medullary thyroid carcinoma) and the lung, such as non-small cell lung cancers (NSCLC). RET has also recently been implicated in the progression of breast and pancreatic tumors, among others. (Mulligan L M, Nat Rev Cancer. 2014, 14(3):173-86.) RET gene abnormalities have also occurred with brain and/or leptomeningeal metastases. RET-rearranged advanced NSCLC patients may have central nervous system (CNS) metastases.

The RET gene has been discovered as an oncogenic driver when activated by gene abnormalities, such as chromosomal rearrangements (RET gene fusions), point mutations, copy number gain, overexpression, or ligand-induced activation. Activated downstream pathways leading to cellular proliferation, migration, and differentiation include the RAS/MEK/ERK, P13K/AKT, JAK/STAT, p38, MAPK, and protein kinase C pathways. The RET kinase domain portion is preserved in fusions, leaving downstream intracellular kinase activity intact. RET gene fusions can occur in NSCLC (incidence 1-2%), papillary thyroid carcinoma (PTC), colorectal cancer (e.g., CCDC6-RET fusion), and breast cancer (e.g., ERC1-RET fusion). RET gene point mutations occur in many inherited forms of medullary thyroid carcinoma (MTC), including multiple endocrine neoplasia 2A (MEN2A), familial medullary thyroid carcinoma (FMTC), and MEN2B. In sporadic MTC, RET mutations are identified in up to 50% of patients. RET gene copy number gain occurs in NSCLC, breast cancer, pancreatic cancer, and glioblastoma. (Ferrara R et al., J Thorac Oncol. 2017, 13(1):27-45; Mulligan L M, Nat Rev Cancer. 2014, 14(3):173-86; Mulligan L M, Front Physiol. 2019, 9:1873.)

RET gene fusions are novel oncogenic drivers in NSCLCs. RET fusions in NSCLC are in a vast number of cases mutually exclusive with mutations in EGFR, KRAS, ALK, HER2, and BRAF, suggesting that RET fusions are independent oncogenic drivers in NSCLC. These recurrent gene fusions were first discovered in late 2011 and have since been confirmed by several independent investigators. (Ju Y S et al., Genome Res. 2012, 22(3):436-45; Suehara Y et al., Clin Cancer Res. 2012, 18(24):6599-608.) The fusions are oncogenic, for example, when expressed in Ba/F3 and NIH-3T3 cells, and these cells can acquire sensitivity to a variety of RET inhibitors, including sorafenib, sunitinib, and vandetanib. (Lipson D et al., Nat Med. 2012, 18(3):382-4; Takeuchi K et al., Nat Med. 2012, 18(3):378-81.)

Multi-kinase inhibitors (MKIs) with activity against RET receptor tyrosine kinase, such as cabozantinib, vandetanib, and lenvatinib, have demonstrated limited efficacy in a minority of patients with medullary thyroid cancers and with RET-fusion NSCLCs. (Drilon A et al., Cancer Discov. 2013, 3(6):630-5; Drilon A et al., Lancet Oncol. 2016, 17(12):1653-60; Drilon A et al., Cancer Discov. 2019, 9(3):384-95.) The magnitude of overall clinical benefit achieved with these MKIs may be lower compared with the outcomes of targeted therapy in patients with different molecular subtypes of NSCLC. Moreover, the risk/benefit profile may be hampered by observations of severe toxicities resulting from more potent inhibition of non-RET kinases, such as VEGFR2. (Ferrara R et al., J Thorac Oncol. 2017, 13(1):27-45.)

Two selective RET inhibitors, selpercatinib (LOXO-292) (NCT03157128) and pralsetinib (BLU-667) (NCT03037385), were approved by the US FDA in 2020 for the treatment of adult patients with metastatic RET fusion-positive non-small cell lung cancer. (Wirth L et al., Ann Oncol. 2019, 30(Suppl 5): v933; Drilon A et al., J Thorac Oncol. 2019, 14(10): S6-S7.) In December 2018 and November 2019, selective RET inhibitors BOS-172738 and TPX-0046 entered the clinical phase. (NCT03780517 and NCT04161391, respectively.) These RET-specific drugs have not been tested extensively in samples or animal models that are resistant to multi-kinase inhibitors, such as cabozantinib, vandetanib, and RXDX-105.

It also remains unclear how effective these RET-specific inhibitors are against lung cancers that have metastasized to the brain. The duration of CNS response as well as the effect on delaying brain metastases occurrence are still unknown. In general, the CNS including the brain is protected by the blood-brain barrier (BBB), a protective endothelial tissue surrounding the CNS, which is a major impediment to the systemic delivery of high molecular weight therapeutic and diagnostic agents to the CNS. Brain penetration of drugs for treating neurological disorders, such as, for example, large biotherapeutic drugs or even low molecular weight drugs with low brain penetration, is limited due, in part, to the extensive and impermeable BBB.

Patients with RET abnormalities have a rare disease with a high unmet medical need. Despite clinical improvement with the introduction of novel targeted therapies, many patients eventually relapse. Patients who progress have limited treatment options; therefore, new agents for treating relapsed patients are still needed. Moreover, limited information is available on the frequency, responsiveness, and overall outcomes in RET-rearranged CNS metastases in advanced NSCLC patients. The frequency of CNS involvement in these patients is 25% at diagnosis, but lifetime prevalence can reach almost a half A low intracranial response has been reported in patients treated with various multi-kinase inhibitors. (Drilon A E et al., J Clin Oncol. 2017, 35(15_Suppl):9069; Gautschi O et al., J Clin Oncol. 2017, 35(13):1403-10.)

Therefore, the availability of a new RET inhibitor able to overcome CNS relapse with a favorable tolerability profile is needed. The availability of a potent and selective RET inhibitor could provide a clinical benefit also for patients naïve to RET-targeted agents, as well as those with resistance mutations. A high CNS penetrance could also represent a substantial clinical improvement in NSCLC patients with brain metastases or leptomeningeal disease.

A RET-specific inhibitor referred to as “HM06” or “TAS0953/HM06” or “HM06/TAS0953” herein is under clinical development. HM06/TAS0953 is a potent and highly selective inhibitor of the phosphorylation of RET. The antitumor efficacy of HM06/TAS0953 in preclinical models support the therapeutic interest of this selective RET inhibitor for clinical application. HM06/TAS0953 inhibited the growth of lung cancer cell lines harboring a RET rearrangement derived from patient samples that were never treated with a RET inhibitor. The results show that HM06/TAS0953 was more effective than RET multi-kinase inhibitors at inhibiting growth of cell lines with RET fusions. HM06/TAS0953 was also tested against cell lines derived from patient samples that were resistant to different RET multi-kinase compounds. The results suggest that HM06/TAS0953 is also effective against cell lines that are resistant to different RET multi-kinase inhibitors and refractory to the inhibitory action of cabozantinib, RXDX-105, and vandetanib.

Additional preclinical data presented herein show that HM06/TAS0953 exhibits inhibitory activity against solid tumor xenografts, including in an orthotopic xenograft model of lung cancer metastasis to the brain. These data indicate that HM06/TAS0953 crosses into the brain and is effective against tumors having RET gene abnormalities in the brain.

HM06/TAS0953 could provide a clinical benefit potentially devoid of adverse reactions resulting from inhibition of non-RET kinases. For example, HM06/TAS0953 may provide an improved treatment and disease management option for NSCLC patients with brain metastases and/or leptomeningeal disease. Additionally, HM06/TAS0953 may be beneficial for patients who are resistant to other RET-kinase or multi-kinase inhibitors (e.g., they have progressed or developed intolerance to the other medication). Considering the limited number of tumor patients carrying the RET gene abnormalities and the rarity of the disease which has a high unmet medical need, patients could benefit from treatment with HM06/TAS0953.

### IV. BRIEF SUMMARY

In accordance with the present invention, compositions and methods for treating patients with cancer having a RET gene abnormality comprising administering HM06/TAS0953 are provided.

The disclosure provides a method of treating a human patient with non-small cell lung cancer (NSCLC) having a RET gene abnormality comprising administering to the human patient a composition comprising 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide, wherein the human patient is administered a dosage equivalent to about 40 mg to about 3000 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day.

The disclosure also provides a method of treating a human patient with locally advanced or metastatic non-small cell lung cancer (NSCLC) having a RET gene abnormality comprising administering to the human patient a composition comprising 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide, wherein the human patient is administered a dosage equivalent to about 40 mg to about 3000 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day.

Further, the disclosure provides a method of treating a human patient with metastatic non-small cell lung cancer (NSCLC) having a RET gene abnormality with brain and/or leptomeningeal metastases comprising administering to the human patient a composition comprising 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide, wherein the human patient is administered an effective amount of 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide.

The disclosure provides a method of treating a human patient with a solid tumor having a RET gene abnormality comprising administering to the human patient a composition comprising 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide, wherein the human patient is administered a dosage equivalent to about 40 mg to about 3000 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day.

The disclosure also provides a method of treating a human patient with a solid tumor having a RET gene abnormality comprising administering to the human patient a composition comprising 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide, wherein the RET gene abnormality comprises a solvent front mutation of a RET protein.

The disclosure also provides a method of treating a human patient with a solid tumor having a RET gene abnormality with brain and/or leptomeningeal metastases comprising administering to the human patient a composition comprising 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide, wherein the human patient is administered an effective amount of 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide.

In some embodiments, the effective amount is a dosage equivalent to about 40 mg to about 3000 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day.

In some embodiments, the brain and/or leptomeningeal metastases is asymptomatic.

In some embodiments, the human patient is administered a dosage equivalent to about 150 mg to about 640 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day. In some embodiments, the human patient is administered a dosage equivalent to about 160 mg to about 640 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day. In some embodiments, the human patient is administered a dosage equivalent to about 320 mg to about 640 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day. In some embodiments, the human patient is administered a dosage equivalent to about 480 mg to about 640 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day. In some embodiments, the human patient is administered a dosage equivalent to about 640 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day.

In some embodiments, the human patient is administered a dosage equivalent to about 480 mg to about 3000 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day. In some embodiments, the human patient is administered a dosage equivalent to about 480 mg to about 2000 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day. In some embodiments, the human patient is administered a dosage equivalent to about 480 mg to about 1500 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day. In some embodiments, the human patient is administered a dosage equivalent to about 480 mg to about 1280 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day. In some embodiments, the human patient is administered a dosage equivalent to about 480 mg to about 1000 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day. In some embodiments, the human patient is administered a dosage equivalent to about 640 mg to about 3000 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day. In some embodiments, the human patient is administered a dosage equivalent to about 640 mg to about 2000 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day. In some embodiments, the human patient is administered a dosage equivalent to about 640 mg to about 1500 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day. In some embodiments, the human patient is administered a dosage equivalent to about 640 mg to about 1280 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day. In some embodiments, the human patient is administered a dosage equivalent to about 640 mg to about 1000 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day. In some embodiments, the human patient is administered a dosage equivalent to about 3000 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day. In some embodiments, the human patient is administered a dosage equivalent to about 2000 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day. In some embodiments, the human patient is administered a dosage equivalent to about 1500 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day. In some embodiments, the human patient is administered a dosage equivalent to about 1280 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day. In some embodiments, the human patient is administered a dosage equivalent to about 1000 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day.

In some embodiments, the human patient is administered a dosage equivalent to about 150 mg to about 500 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day. In some embodiments, the human patient is administered a dosage equivalent to about 160 mg to about 500 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day. In some embodiments, the human patient is administered a dosage equivalent to about 150 mg or about 160 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day.

In some embodiments, the composition is administered orally. In some embodiments, the composition is administered orally as a single tablet or multiple tablets. In some embodiments, each tablet comprises a dose equivalent to about 10 or about 50 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base.

In some embodiments, the composition comprises the di-hydrochloride salt of 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide.

In some embodiments, the composition further comprises citric acid, microcrystalline cellulose, lactose, polyvinyl N-pyrrolidone, sodium lauryl sulfate, and/or glyceryl behenate.

In some embodiments, the composition is administered once per day (QD) or twice per day (BID). In some embodiments, the composition is administered twice per day (BID).

In some embodiments, the human patient is administered a dosage equivalent to about 160 to about 1500 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base twice per day (BID). In some embodiments, the human patient is administered a dosage equivalent to about 160 to about 1000 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base twice per day (BID). In some embodiments, the human patient is administered a dosage equivalent to about 160 to about 750 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base twice per day (BID). In some embodiments, the human patient is administered a dosage equivalent to about 160 to about 640 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base twice per day (BID). In some embodiments, the human patient is administered a dosage equivalent to about 160 to about 500 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base twice per day (BID). In some embodiments, the human patient is administered a dosage equivalent to about 160 to about 320 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base twice per day (BID).

In some embodiments, the human patient is administered a dosage equivalent to about 320 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base twice per day (BID). In some embodiments, the human patient is administered a dosage equivalent to about 500 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base twice per day (BID). In some embodiments, the human patient is administered a dosage equivalent to about 640 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base twice per day (BID). In some embodiments, the human patient is administered a dosage equivalent to about 750 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base twice per day (BID). In some embodiments, the human patient is administered a dosage equivalent to about 1000 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base twice per day (BID). In some embodiments, the human patient is administered a dosage equivalent to about 1500 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base twice per day (BID).

In some embodiments, the dosage is the same for a patient weighing greater than 50 kg and for a patient weighing less than 50 kg.

In some embodiments, the composition is administered in at least one 21-day treatment cycle.

In some embodiments, the RET gene abnormality comprises at least one of a RET gene fusion, a point mutation, a deletion mutation, an increased copy number of the RET gene, overexpression of any one or more thereof, and overexpression of a RET gene. In some embodiments, the RET gene abnormality comprises a RET gene fusion. In some embodiments, the RET gene abnormality comprises a RET gene fusion with CCDC6, KIF5B, or TRIM33.

In some embodiments, the RET gene abnormality comprises a resistance mutation of a RET protein. In some embodiments, the RET gene abnormality comprises a solvent front mutation of a RET protein and/or a mutation in the hinge region of a RET protein.

In some embodiments, the RET gene abnormality comprises a mutation of a RET protein at amino acid residue 730, 736, 760, 772, 804, 806, 807, 808, 809, 810, and/or 883. In some embodiments, the RET gene abnormality comprises a mutation of a RET protein at amino acid residue 804, 806, 807, 808, 809, and/or 810. In some embodiments, the RET gene abnormality comprises a mutation of a RET protein at amino acid residue 810. In some embodiments, the RET gene abnormality comprises a mutation of a RET protein comprising: a) a V804X mutation, wherein X is any amino acid other than valine or glutamic acid; b) a Y806X mutation, wherein X is any amino acid other than tyrosine; c) a A807X mutation, wherein X is any amino acid other than alanine; d) a K808X mutation, wherein X is any amino acid other than alanine; e) a Y809X mutation, wherein X is any amino acid other than tyrosine; and/or f) a G810X mutation, wherein X is any amino acid other than glycine. In some embodiments, the RET gene abnormality comprises a mutation of a RET protein comprising: a) a L730Q or L730R mutation; b) a G736A mutation; c) a L760Q mutation; d) a L772M mutation; e) a V804L or V804M mutation; f) a Y806C, Y806S, Y806H, or Y806N mutation; g) a G810R, G810S, G810C, G810V, G810D, or G810A; and/or a A883V mutation. In some embodiments, the RET gene abnormality comprises a mutation of a RET protein comprising: a) a V804L or V804M mutation; b) a Y806C, Y806S, Y806H, or Y806N mutation; and/or c) a G810R, G810S, G810C, G810V, G810D, or G810A mutation. In some embodiments, the RET gene abnormality comprises a G810R, G810S, G810C, G810V, G810D, or G810A mutation of a RET protein. In some embodiments, the RET gene abnormality comprises a G810R mutation of a RET protein.

In some embodiments, the cancer or the tumor is resistant to at least one multi-kinase inhibitor. In some embodiments, the cancer or the tumor is resistant to at least one RET selective inhibitor. In some embodiments, the cancer or the tumor is not resistant to 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide and is resistant to at least one other RET selective inhibitor. In some embodiments, the cancer or the tumor is resistant to selpercatinib and/or pralsetinib. In some embodiments, the cancer or the tumor comprises cells resistant to selpercatinib and/or pralsetinib.

In some embodiments, the human patient previously received prior treatment for the cancer or the tumor. In some embodiments, the cancer or tumor being treated progressed following a prior treatment for the cancer or tumor. In some embodiments, the human patient developed intolerance to a prior treatment for the cancer or tumor. In some embodiments, the human patient previously received a multi-kinase inhibitor. In some embodiments, the human patient previously received cabozantinib, vandetanib, lenvatinib, and/or RXDX-105. In some embodiments, the human patient previously received a RET selective inhibitor. In some embodiments, the human patient previously received selpercatinib and/or pralsetinib. In some embodiments, the human patient has not previously received a RET selective inhibitor.

In some embodiments, the human patient has at least one of salivary gland cancer, lung cancer, colorectal cancer, thyroid cancer, breast cancer, pancreatic cancer, ovarian cancer, skin cancer, and brain cancer. In some embodiments, the human patient has at least one of medullary or anaplastic thyroid cancer, metastatic breast cancer, and metastatic pancreatic adenocarcinoma.

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### V. BRIEF DESCRIPTION OF THE DRAWINGS

FIGS. 1A, 1B, 1C, and 1D describe the efficacy of HM06/TAS0953 in a brain metastasis model harboring a KIF5B-RET Luc fusion. FIG. 1A shows antitumor efficacy of HM06/TAS0953 compared to vehicle control when administered at 50 mg/kg BID. FIG. 1B shows percent body weight change in the KIFSB-RET Luc fusion brain metastasis mice treated with HM06/TAS0953 compared to the vehicle control mice. FIG. 1C shows a higher survival rate in the HM06/TAS0953 group than the vehicle control group. FIG. 1D shows IVIS image and pathology in HM06/TAS0953-treated mice brains compared to control.

FIG. 2 shows the effects of HM06/TAS0953 on KIFSB-RET fusion tumor volume in a vandetanib-refractory mouse model compared to continuous vandetanib treatment.

FIGS. 3A, 3B, 3C, 3D, 3E, and 3F describe the efficacy of HM06/TAS0953 at inhibiting growth of RET-fusion positive cell lines in comparison with three RET multi-kinase inhibitors: cabozantinib, RXDX-105, and vandetanib. FIG. 3A shows treatment on a treatment naïve cell line derived from a sample obtained from a patient who had never been treated with an anti-cancer therapy (KIFSB-RET). FIG. 3B shows treatment on a CCDC6-RET fusion cell line. FIG. 3C shows effect on the isogenic counterpart of the same cell line that expresses the empty control plasmid. FIG. 3D shows treatment on a TRIM33-RET fusion cell line. FIG. 3E shows treatment on a cell line derived from a sample obtained from a patient who was resistant to cabozantinib (CCDC6-RET). FIG. 3F shows treatment on a cell line, obtained from a patient resistant to RXDX-105.

FIGS. 4A, 4B, and 4C describe efficacy of HM06/TAS0953 on growth of 3T3-CCDC6-RET xenograft tumors. FIG. 4A shows the change in volume of each individual tumor from the beginning of treatment to the end. Results are the mean±SE of 5 independent tumors at each time point. FIG. 4B shows the change in volume of each tumor. *P<0.05, compared to vehicle treated group. FIG. 4C shows animal weight as a function of time. Results are the mean SE of 5 animals per group.

FIGS. 5A, 5B, and 5C describe efficacy of HM06/TAS0953 on growth of xenograft tumors (TRIM33-RET fusion). FIG. 5A shows tumor volume as a function of time. Results are the mean±SE of 5 independent tumors at each time point. FIG. 5B shows the change in volume of each individual tumor from the beginning of treatment to the end. There was a significant reduction in average tumor volume in all groups, compared to the vehicle-treated group (p<0.05). FIG. 5C shows animal weight as a function of time. Results are the mean±SE of 5 animals per group. There was a significant reduction in animal weight in the vandetanib-treated group at all time points after starting treatment (p<0.05).

FIGS. 6A, 6B, and 6C describe efficacy of HM06/TAS0953 on growth of PDX tumors derived from tumor samples obtained from a patient who was no longer responding to cabozantinib (CCDC6-RET). FIG. 6A shows tumor volume as a function of time. Results are the mean±SE of 5 independent tumors at each time point. FIG. 6B shows the change in volume of each individual tumor from the beginning of treatment to the end. There was a significant reduction in tumor volume in all groups, compared to the vehicle-treated group (p<0.05). FIG. 6C shows animal weight as a function of time. Results are the mean±SE of 8 animals per group.

FIGS. 7A, 7B, and 7C describe efficacy of HM06/TAS0953 on growth of PDX tumors derived from a patient who was resistant to RXDX-105 therapy (CCDC6-RET). FIG. 7A shows tumor volume as a function of time. Results are the mean±SE of 5 independent tumors at each time point. FIG. 7B shows the change in volume of each individual tumor from the beginning of treatment to the end. There was a significant reduction in tumor volume in all groups, compared to the vehicle-treated group (p<0.05). FIG. 7C shows animal weight as a function of time. Results are the mean±SE of 5 animals per group.

FIGS. 8A, 8B, and 8C describe efficacy of HM06/TAS0953 on growth of PDX tumors derived from a patient who had a poor response to RXDX-105 (CCDC6-RET). FIG. 8A shows tumor volume as a function of time. Results are the mean±SE of 5 independent tumors at each time point. There was a significant reduction in tumor volume in all groups, compared to the vehicle-treated group (p<0.05). FIG. 8B shows the change in volume of each individual tumor from the beginning of treatment to the end. One tumor in the HM06/TAS0953 100 mg/kg QD group increased by only 20.7%. FIG. 8C shows animal weight as a function of time. Results are the mean±SE of 5 animals per group.

FIGS. 9A, 9B, and 9C describe efficacy of HM06/TAS0953 on growth of tumors implanted into the brain of mice (TRIM33-RET fusion). FIG. 9A shows images of bioluminescence signals at the beginning and end of treatment. FIG. 9B shows quantitation of luminescence (left) and animal weight measurements (right). There was a significant reduction in tumor volume, compared to the vehicle-treated group (p<0.05). Results represent mean±SE of 5 (vehicle) or 4 (HM06) animals. FIG. 9C shows a Kaplan-Meier plot showing survival of tumor-bearing mice throughout the study.

FIGS. 10A, 10B, 10C, and 10D show efficacy of HM06/TAS0953, vandetanib and LOXO-292 on growth of tumors implanted into the brain of mice (TRIM33-RET fusion). FIG. 10A shows representative images of bioluminescence 33 and 93 days after implantation of cells. FIG. 10B shows quantitation of luminescence signals. Results represent mean±SE of 6 animals per group. FIG. 10C shows Kaplan-Meier plot showing survival of tumor-bearing mice throughout the study. The adjusted p values for comparison of HM06- and LOXO-292-treated groups are given below the graph. FIG. 10D shows animal weight over treatment time.

FIGS. 11A, 111B, and 11C show the pharmacokinetic profiles of HM06/TAS0953 in rat. FIG. 11A shows plasma concentration over time after single oral administration of HM06/TAS0953 at 3, 10, 30, and 50 mg/kg. FIG. 11B shows plasma concentration over time after a single 3 mg/kg IV administration of HM06/TAS0953. FIG. 11C shows the pharmacokinetic profile of HM06/TAS0953 in PFC, CSF and plasma (total and free fraction) of freely-moving adult male Han® Wistar rats after oral administration of 10 mg/kg. A free plasma to free brain concentration ratio 1:1 indicates that HM06/TAS0953 freely crosses the blood brain barrier.

FIGS. 12A, 12B, and 12C show x-ray crystal structures of wild type RET complexes with TAS compound 1, BLU-667, and LOXO-292 in the region of RET amino acid residues 806-810. FIG. 12A shows based on co-crystal structural data, BLU-667 and LOXO-292 bind to the same pocket in RET (Pocket B) whereas TAS compound 1 binds to a different pocket in RET (Pocket A) with a different binding mode. FIG. 12B shows co-crystal complexes of RET and TAS compound 1, BLU-667, and LOXO-292 and FIG. 12C shows the co-crystal complex of RET and TAS compound 1.

FIGS. 13A, 13B, and 13C show the effects of HM06/TAS0953, LOXO-292, and BLU-667 in a Ba/F3 KIF5B-RET^{G810R} cell-bearing nude mouse model. FIG. 13A shows the effects on tumor volume where HM06/TAS0953, LOXO-292, and BLU-667 are each administered at a twice-daily dose of 10 mg/kg. FIG. 13B shows the effects at a twice-daily dose of 30 mg/kg. FIG. 13C shows the change of body weight during the treatment period in the Ba/F3 KIF5B-RET^{G810R} cell-bearing nude mice.

FIG. 14A and 14B show the effects the effects of HM06/TAS0953, LOXO-292, and BLU-667 in a Ba/F3 KIF5B-RET^{G810R} cell-bearing nude mouse model. FIG. 14A shows the effects on tumor volume where HM06/TAS0953 is administered at a twice-daily dose of 50 mg/kg, and LOXO-292 and BLU-667 are each administered at a twice-daily dose of 30 mg/kg. FIG. 14B shows the change of body weight during the treatment period in the Ba/F3 KIF5B-RET^{G810R} cell-bearing nude mice.

FIGS. 15A and 15B show the phosphorylation of RET in Ba/F3 KIFSB-RET^{G810R}tumors one hour after administration of HM06/TAS0953, BLU-667, and LOXO-292. Mice bearing Ba/F3 KIF5B-RET^{G810R} were orally administered HM06/TAS0953 at 10, 30, or 50 mg/kg or LOXO292 and BLU667 at 10 or 30 mg/kg, respectively once. The tumors were collected and lysed at 1 hour post dosing. The cell lysates were immunoblotted to detect the indicated proteins.

### DESCRIPTION OF CERTAIN SEQUENCES

TABLE 1
Table 1 provides a listing of certain sequences referenced herein.
Description of Certain Sequences

| SEQ | | |
|---|---|---|
| ID NO: | SEQUENCE | DESCRIPTION |
| 1 | agtcccgcga ccgaagcagg gcgcgcagca gcgctgagtg | Exemplary homo |
| | ccccggaacg tgcgtcgcgc ccccagtgtc cgtcgcgtcc | sapiens ret proto- |
| | gccgcgcccc gggcggggat ggggcggcca gactgagcgc | oncogene (RET) |
| | cgcacccgcc atccagaccc gccggcccta gccgcagtcc | mRNA sequence |
| | ctccagccgt ggccccagcg cgcacgggcg atggcgaagg | NCBI Reference |
| | cgacgtccgg tgccgcgggg ctgcgtctgc tgttgctgct | Sequence: |
| | gctgctgccg ctgctaggca aagtggcatt gggcctctac | NM_020975.6 |
| | ttctcgaggg atgcttactg ggagaagctg tatgtggacc | |
| | aggcagccgg cacgcccttg ctgtacgtcc atgccctgcg | |
| | ggacgcccct gaggaggtgc ccagcttccg cctgggccag | |
| | catctctacg gcacgtaccg cacacggctg catgagaaca | |
| | actggatctg catccaggag gacaccggcc tcctctacct | |
| | taaccggagc ctggaccata gctcctggga gaagctcagt | |
| | gtccgcaacc gcggctttcc cctgctcacc gtctacctca | |
| | aggtcttcct gtcacccaca tcccttcgtg agggcgagtg | |
| | ccagtggcca ggctgtgccc gcgtatactt ctccttcttc | |
| | aacacctcct ttccagcctg cagctccctc aagccccggg | |
| | agctctgctt cccagagaca aggccctcct tccgcattcg | |
| | ggagaaccga cccccaggca ccttccacca gttccgcctg | |
| | ctgcctgtgc agttcttgtg ccccaacatc agcgtggcct | |
| | acaggctcct ggagggtgag ggtctgccct tccgctgcgc | |
| | cccggacagc ctggaggtga gcacgcgctg ggccctggac | |
| | cgcgagcagc gggagaagta cgagctggtg gccgtgtgca | |
| | ccgtgcacgc cggcgcgcgc gaggaggtgg tgatggtgcc | |
| | cttcccggtg accgtgtacg acgaggacga ctcggcgccc | |
| | accttccccg cgggcgtcga caccgccagc gccgtggtgg | |
| | agttcaagcg gaaggaggac accgtggtgg ccacgctgcg | |
| | tgtcttcgat gcagacgtgg tacctgcatc aggggagctg | |
| | gtgaggcggt acacaagcac gctgctcccc ggggacacct | |
| | gggcccagca gaccttccgg gtggaacact ggcccaacga | |
| | gacctcggtc caggccaacg gcagcttcgt gcgggcgacc | |
| | gtacatgact ataggctggt tctcaaccgg aacctctcca | |
| | tctcggagaa ccgcaccatg cagctggcgg tgctggtcaa | |
| | tgactcagac ttccagggcc caggagcggg cgtcctcttg | |
| | ctccacttca acgtgtcggt gctgccggtc agcctgcacc | |
| | tgcccagtac ctactccctc tccgtgagca ggagggctcg | |
| | ccgatttgcc cagatcggga aagtctgtgt ggaaaactgc | |
| | caggcattca gtggcatcaa cgtccagtac aagctgcatt | |
| | cctctggtgc caactgcagc acgctagggg tggtcacctc | |
| | agccgaggac acctcgggga tcctgtttgt gaatgacacc | |
| | aaggccctgc ggcggcccaa gtgtgccgaa cttcactaca | |
| | tggtggtggc caccgaccag cagacctcta ggcaggccca | |
| | ggcccagctg cttgtaacag tggaggggtc atatgtggcc | |
| | gaggaggcgg gctgccccct gtcctgtgca gtcagcaaga | |
| | gacggctgga gtgtgaggag tgtggcggcc tgggctcccc | |
| | aacaggcagg tgtgagtgga ggcaaggaga tggcaaaggg | |
| | atcaccagga acttctccac ctgctctccc agcaccaaga | |
| | cctgccccga cggccactgc gatgttgtgg agacccaaga | |
| | catcaacatt tgccctcagg actgcctccg gggcagcatt | |
| | gttgggggac acgagcctgg ggagccccgg gggattaaag | |
| | ctggctatgg cacctgcaac tgcttccctg aggaggagaa | |
| | gtgcttctgc gagcccgaag acatccagga tccactgtgc | |
| | gacgagctgt gccgcacggt gatcgcagcc gctgtcctct | |
| | tctccttcat cgtctcggtg ctgctgtctg ccttctgcat | |
| | ccactgctac cacaagtttg cccacaagcc acccatctcc | |
| | tcagctgaga tgaccttccg gaggcccgcc caggccttcc | |
| | cggtcagcta ctcctcttcc ggtgcccgcc ggccctcgct | |
| | ggactccatg gagaaccagg tctccgtgga tgccttcaag | |
| | atcctggagg atccaaagtg ggaattccct cggaagaact | |
| | tggttcttgg aaaaactcta ggagaaggcg aatttggaaa | |
| | agtggtcaag gcaacggcct tccatctgaa aggcagagca | |
| | gggtacacca cggtggccgt gaagatgctg aaagagaacg | |
| | cctccccgag tgagctgcga gacctgctgt cagagttcaa | |
| | cgtcctgaag caggtcaacc acccacatgt catcaaattg | |
| | tatggggcct gcagccagga tggcccgctc ctcctcatcg | |
| | tggagtacgc caaatacggc tccctgcggg gcttcctccg | |
| | cgagagccgc aaagtggggc ctggctacct gggcagtgga | |
| | ggcagccgca actccagctc cctggaccac ccggatgagc | |
| | gggccctcac catgggcgac ctcatctcat ttgcctggca | |
| | gatctcacag gggatgcagt atctggccga gatgaagctc | |
| | gttcatcggg acttggcagc cagaaacatc ctggtagctg | |
| | aggggcggaa gatgaagatt tcggatttcg gcttgtcccg | |
| | agatgtttat gaagaggatt cctacgtgaa gaggagccag | |
| | ggtcggattc cagttaaatg gatggcaatt gaatcccttt | |
| | ttgatcatat ctacaccacg caaagtgatg tatggtcttt | |
| | tggtgtcctg ctgtgggaga tcgtgaccct agggggaaac | |
| | ccctatcctg ggattcctcc tgagcggctc ttcaaccttc | |
| | tgaagaccgg ccaccggatg gagaggccag acaactgcag | |
| | cgaggagatg taccgcctga tgctgcaatg ctggaagcag | |
| | gagccggaca aaaggccggt gtttgcggac atcagcaaag | |
| | acctggagaa gatgatggtt aagaggagag actacttgga | |
| | ccttgcggcg tccactccat ctgactccct gatttatgac | |
| | gacggcctct cagaggagga gacaccgctg gtggactgta | |
| | ataatgcccc cctccctcga gccctccctt ccacatggat | |
| | tgaaaacaaa ctctatggca tgtcagaccc gaactggcct | |
| | ggagagagtc ctgtaccact cacgagagct gatggcacta | |
| | acactgggtt tccaagatat ccaaatgata gtgtatatgc | |
| | taactggatg ctttcaccct cagcggcaaa attaatggac | |
| | acgtttgata gttaacattt ctttgtgaaa ggtaatggac | |
| | tcacaagggg aagaaacatg ctgagaatgg aaagtctacc | |
| | ggccctttct ttgtgaacgt cacattggcc gagccgtgtt | |
| | cagttcccag gtggcagact cgtttttggt agtttgtttt | |
| | aacttccaag gtggttttac ttctgatagc cggtgatttt | |
| | ccctcctagc agacatgcca caccgggtaa gagctctgag | |
| | tcttagtggt taagcattcc tttctcttca gtgcccagca | |
| | gcacccagtg ttggtctgtg tccatcagtg accaccaaca | |
| | ttctgtgttc acatgtgtgg gtccaacact tactacctgg | |
| | tgtatgaaat tggacctgaa ctgttggatt tttctagttg | |
| | ccgccaaaca aggcaaaaaa atttaaacat gaagcacaca | |
| | cacaaaaaag gcagtaggaa aaatgctggc cctgatgacc | |
| | tgtccttatt cagaatgaga gactgcgggg ggggcctggg | |
| | ggtagtgtca atgcccctcc agggctggag gggaagaggg | |
| | gccccgagga tgggcctggg ctcagcattc gagatcttga | |
| | gaatgatttt ttttaaatca tgcaaccttt ccttaggaag | |
| | acatttggtt ttcatcatga ttaagatgat tcctagattt | |
| | agcacaatgg agagattcca tgccatcttt actatgtgga | |
| | tggtggtatc agggaagagg gctcacaaga cacatttgtc | |
| | ccccgggccc accacatcat cctcacgtgt tcggtactga | |
| | gcagccacta cccctgatga gaacagtatg aagaaagggg | |
| | gctgttggag tcccagaatt gctgacagca gaggctttgc | |
| | tgctgtgaat cccacctgcc accagcctgc agcacacccc | |
| | acagccaagt agaggcgaaa gcagtggctc atcctacctg | |
| | ttaggagcag gtagggcttg tactcacttt aatttgaatc | |
| | ttatcaactt actcataaag ggacaggcta gctagctgtg | |
| | ttagaagtag caatgacaat gaccaaggac tgctacacct | |
| | ctgattacaa ttctgatgtg aaaaagatgg tgtttggctc | |
| | ttatagagcc tgtgtgaaag gcccatggat cagctcttcc | |
| | tgtgtttgta atttaatgct gctacaagat gtttctgttt | |
| | cttagattct gaccatgact cataagcttc ttgtcattct | |
| | tcattgcttg tttgtggtca cagatgcaca acactcctcc | |
| | agtcttgtgg gggcagcttt tgggaagtct cagcagctct | |
| | tctggctgtg ttgtcagcac tgtaacttcg cagaaaagag | |
| | tcggattacc aaaacactgc ctgctcttca gacttaaagc | |
| | actgatagga cttaaaatag tctcattcaa atactgtatt | |
| | ttatataggc atttcacaaa aacagcaaaa ttgtggcatt | |
| | ttgtgaggcc aaggcttgga tgcgtgtgta atagagcctt | |
| | gtggtgtgtg cgcacacacc cagagggaga gtttgaaaaa | |
| | tgcttattgg acacgtaacc tggctctaat ttgggctgtt | |
| | tttcagatac actgtgataa gttcttttac aaatatctat | |
| | agacatggta aacttttggt tttcagatat gcttaatgat | |
| | agtcttacta aatgcagaaa taagaataaa ctttctcaaa | |
| | ttattaaaaa tgcctacaca gtaagtgtga attgctgcaa | |
| | caggtttgtt ctcaggaggg taagaactcc aggtctaaac | |
| | agctgaccca gtgatgggga atttatcctt gaccaattta | |
| | tccttgacca ataacctaat tgtctattcc tgagttataa | |
| | aagtccccat ccttattagc tctactggaa ttttcataca | |
| | cgtaaatgca gaagttacta agtattaagt attactgagt | |
| | attaagtagt aatctgtcag ttattaaaat ttgtaaaatc | |
| | tatttatgaa aggtcattaa accagatcat gttccttttt | |
| | ttgtaatcaa ggtgactaag aaaatcagtt gtgtaaataa | |
| | aatcatgtat cataaaa | |
| 2 | MAKATSGAAGLRLLLLLLLPLLGKVALGLYFSRDAYWEKLYVDQAA | Exemplary homo |
| | GTPLLYVHALRDAPEEVPSFRLGQHLYGTYRTRLHENNWICIQEDT | sapiens ret proto- |
| | GLLYLNRSLDHSSWEKLSVRNRGFPLLTVYLKVELSPTSLREGECQ | oncogene (RET) |
| | WPGCARVYFSFFNTSFPACSSLKPRELCFPETRPSFRIRENRPPGT | amino acid |
| | FHQFRLLPVQFLCPNISVAYRLLEGEGLPERCAPDSLEVSTRWALD | sequence |
| | REQREKYELVAVCTVHAGAREEVVMVPFPVTVYDEDDSAPTEPAGV | NCBI Reference |
| | DTASAVVEFKRKEDTVVATLRVEDADVVPASGELVRRYTSTLLPGD | Sequence: |
| | TWAQQTERVEHWPNETSVQANGSFVRATVHDYRLVLNRNLSISENR | NM 020975.6 |
| | TMQLAVLVNDSDFQGPGAGVLLLHENVSVLPVSLHLPSTYSLSVSR | |
| | RARRFAQIGKVCVENCQAFSGINVQYKLHSSGANCSTLGVVTSAED | |
| | TSGILFVNDTKALRRPKCAELHYMVVATDQQTSRQAQAQLLVTVEG | |
| | SYVAEEAGCPLSCAVSKRRLECEECGGLGSPTGRCEWRQGDGKGIT | |
| | RNFSTCSPSTKTCPDGHCDVVETQDINICPQDCLRGSIVGGHEPGE | |
| | PRGIKAGYGTCNCFPEEEKCFCEPEDIQDPLCDELCRTVIAAAVLE | |
| | SFIVSVLLSAFCIHCYHKFAHKPPISSAEMTERRPAQAFPVSYSSS | |
| | GARRPSLDSMENQVSVDAFKILEDPKWEFPRKNLVLGKTLGEGEFG | |
| | KVVKATAFHLKGRAGYTTVAVKMLKENASPSELRDLLSEENVLKQV | |
| | NHPHVIKLYGACSQDGPLLLIVEYAKYGSLRGFLRESRKVGPGYLG | |
| | SGGSRNSSSLDHPDERALTMGDLISFAWQISQGMQYLAEMKLVHRD | |
| | LAARNILVAEGRKMKISDFGLSRDVYEEDSYVKRSQGRIPVKWMAI | |
| | ESLFDHIYTTQSDVWSFGVLLWEIVTLGGNPYPGIPPERLENLLKT | |
| | GHRMERPDNCSEEMYRLMLQCWKQEPDKRPVFADISKDLEKMMVKR | |
| | RDYLDLAASTPSDSLIYDDGLSEEETPLVDCNNAPLPRALPSTWIE | |
| | NKLYGMSDPNWPGESPVPLTRADGTNTGFPRYPNDSVYANWMLSPS | |
| | AAKLMDTEDS | |
| 3 | MAKATSGAAGLRLLLLLLLPLLGKVALGLYFSRDAYWEKLYVDQAA | Exemplary RET |
| | GTPLLYVHALRDAPEEVPSFRLGQHLYGTYRTRLHENNWICIQEDT | protein with |
| | GLLYLNRSLDHSSWEKLSVRNRGFPLLTVYLKVELSPTSLREGECQ | V804X mutation, |
| | WPGCARVYFSFENTSFPACSSLKPRELCFPETRPSFRIRENRPPGT | wherein X is any |
| | FHQFRLLPVQFLCPNISVAYRLLEGEGLPERCAPDSLEVSTRWALD | amino acid other |
| | REQREKYELVAVCTVHAGAREEVVMVPFPVTVYDEDDSAPTFPAGV | than valine or |
| | DTASAVVEFKRKEDTVVATLRVEDADVVPASGELVRRYTSTLLPGD | glutamic acid. |
| | TWAQQTERVEHWPNETSVQANGSFVRATVHDYRLVLNRNLSISENR | |
| | TMQLAVLVNDSDFQGPGAGVLLLHENVSVLPVSLHLPSTYSLSVSR | |
| | RARRFAQIGKVCVENCQAFSGINVOYKLHSSGANCSTLGVVTSAED | |
| | TSGILFVNDTKALRRPKCAELHYMVVATDQQTSRQAQAQLLVTVEG | |
| | SYVAEEAGCPLSCAVSKRRLECEECGGLGSPTGRCEWRQGDGKGIT | |
| | RNFSTCSPSTKTCPDGHCDVVETQDINICPQDCLRGSIVGGHEPGE | |
| | PRGIKAGYGTCNCFPEEEKCFCEPEDIQDPLCDELCRTVIAAAVLF | |
| | SFIVSVLLSAFCIHCYHKFAHKPPISSAEMTERRPAQAFPVSYSSS | |
| | GARRPSLDSMENQVSVDAFKILEDPKWEFPRKNLVLGKTLGEGEFG | |
| | KVVKATAFHLKGRAGYTTVAVKMLKENASPSELRDLLSEENVLKQV | |
| | NHPHVIKLYGACSQDGPLLLIXEYAKYGSLRGELRESRKVGPGYLG | |
| | SGGSRNSSSLDHPDERALTMGDLISFAWQISQGMQYLAEMKLVHRD | |
| | LAARNILVAEGRKMKISDFGLSRDVYEEDSYVKRSQGRIPVKWMAI | |
| | ESLFDHIYTTQSDVWSFGVLLWEIVTLGGNPYPGIPPERLENLLKT | |
| | GHRMERPDNCSEEMYRLMLQCWKQEPDKRPVFADISKDLEKMMVKR | |
| | RDYLDLAASTPSDSLIYDDGLSEEETPLVDCNNAPLPRALPSTWIE | |
| | NKLYGMSDPNWPGESPVPLTRADGTNTGFPRYPNDSVYANWMLSPS | |
| | AAKLMDTEDS | |
| 4 | MAKATSGAAGLRLLLLLLLPLLGKVALGLYFSRDAYWEKLYVDQAA | Exemplary RET |
| | GTPLLYVHALRDAPEEVPSFRLGQHLYGTYRTRLHENNWICIQEDT | protein with |
| | GLLYLNRSLDHSSWEKLSVRNRGFPLLTVYLKVELSPTSLREGECQ | Y806X mutation, |
| | WPGCARVYFSFENTSFPACSSLKPRELCFPETRPSFRIRENRPPGT | wherein X is any |
| | FHQFRLLPVQFLCPNISVAYRLLEGEGLPERCAPDSLEVSTRWALD | amino acid other |
| | REQREKYELVAVCTVHAGAREEVVMVPFPVTVYDEDDSAPTEPAGV | than tyrosine. |
| | DTASAVVEFKRKEDTVVATLRVEDADVVPASGELVRRYTSTLLPGD | |
| | TWAQQTERVEHWPNETSVQANGSFVRATVHDYRLVLNRNLSISENR | |
| | TMQLAVLVNDSDFQGPGAGVLLLHFNVSVLPVSLHLPSTYSLSVSR | |
| | RARRFAQIGKVCVENCQAFSGINVQYKLHSSGANCSTLGVVTSAED | |
| | TSGILFVNDTKALRRPKCAELHYMVVATDQQTSRQAQAQLLVTVEG | |
| | SYVAEEAGCPLSCAVSKRRLECEECGGLGSPTGRCEWRQGDGKGIT | |
| | RNFSTCSPSTKTCPDGHCDVVETQDINICPQDCLRGSIVGGHEPGE | |
| | PRGIKAGYGTCNCFPEEEKCFCEPEDIQDPLCDELCRTVIAAAVLF | |
| | SFIVSVLLSAFCIHCYHKFAHKPPISSAEMTERRPAQAFPVSYSSS | |
| | GARRPSLDSMENQVSVDAFKILEDPKWEFPRKNLVLGKTLGEGEFG | |
| | KVVKATAFHLKGRAGYTTVAVKMLKENASPSELRDLLSEENVLKQV | |
| | NHPHVIKLYGACSQDGPLLLIVEXAKYGSLRGFLRESRKVGPGYLG | |
| | SGGSRNSSSLDHPDERALTMGDLISFAWQISQGMQYLAEMKLVHRD | |
| | LAARNILVAEGRKMKISDFGLSRDVYEEDSYVKRSQGRIPVKWMAI | |
| | ESLEDHIYTTQSDVWSFGVLLWEIVTLGGNPYPGIPPERLENLLKT | |
| | GHRMERPDNCSEEMYRLMLQCWKQEPDKRPVFADISKDLEKMMVKR | |
| | RDYLDLAASTPSDSLIYDDGLSEEETPLVDCNNAPLPRALPSTWIE | |
| | NKLYGMSDPNWPGESPVPLTRADGTNTGFPRYPNDSVYANWMLSPS | |
| | AAKLMDTFDS | |
| 5 | MAKATSGAAGLRLLLLLLLPLLGKVALGLYFSRDAYWEKLYVDQAA | Exemplary RET |
| | GTPLLYVHALRDAPEEVPSFRLGQHLYGTYRTRLHENNWICIQEDT | protein with |
| | GLLYLNRSLDHSSWEKLSVRNRGFPLLTVYLKVELSPTSLREGECQ | A807X mutation, |
| | WPGCARVYFSFFNTSFPACSSLKPRELCFPETRPSFRIRENRPPGT | wherein X is any |
| | FHQFRLLPVQFLCPNISVAYRLLEGEGLPERCAPDSLEVSTRWALD | amino acid other |
| | REQREKYELVAVCTVHAGAREEVVMVPFPVTVYDEDDSAPTFPAGV | than alanine. |
| | DTASAVVEFKRKEDTVVATLRVEDADVVPASGELVRRYTSTLLPGD | |
| | TWAQQTERVEHWPNETSVQANGSFVRATVHDYRLVLNRNLSISENR | |
| | TMQLAVLVNDSDFQGPGAGVLLLHENVSVLPVSLHLPSTYSLSVSR | |
| | RARRFAQIGKVCVENCQAFSGINVQYKLHSSGANCSTLGVVTSAED | |
| | TSGILFVNDTKALRRPKCAELHYMVVATDQQTSRQAQAQLLVTVEG | |
| | SYVAEEAGCPLSCAVSKRRLECEECGGLGSPTGRCEWRQGDGKGIT | |
| | RNFSTCSPSTKTCPDGHCDVVETQDINICPQDCLRGSIVGGHEPGE | |
| | PRGIKAGYGTCNCFPEEEKCFCEPEDIQDPLCDELCRTVIAAAVLF | |
| | SFIVSVLLSAFCIHCYHKFAHKPPISSAEMTERRPAQAFPVSYSSS | |
| | GARRPSLDSMENQVSVDAFKILEDPKWEFPRKNLVLGKTLGEGEFG | |
| | KVVKATAFHLKGRAGYTTVAVKMLKENASPSELRDLLSEFNVLKQV | |
| | NHPHVIKLYGACSQDGPLLLIVEYXKYGSLRGELRESRKVGPGYLG | |
| | SGGSRNSSSLDHPDERALTMGDLISFAWQISQGMQYLAEMKLVHRD | |
| | LAARNILVAEGRKMKISDFGLSRDVYEEDSYVKRSQGRIPVKWMAI | |
| | ESLFDHIYTTQSDVWSFGVLLWEIVTLGGNPYPGIPPERLENLLKT | |
| | GHRMERPDNCSEEMYRLMLQCWKQEPDKRPVFADISKDLEKMMVKR | |
| | RDYLDLAASTPSDSLIYDDGLSEEETPLVDCNNAPLPRALPSTWIE | |
| | NKLYGMSDPNWPGESPVPLTRADGTNTGFPRYPNDSVYANWMLSPS | |
| | AAKLMDTEDS | |
| 6 | MAKATSGAAGLRLLLLLLLPLLGKVALGLYFSRDAYWEKLYVDQAA | Exemplary RET |
| | GTPLLYVHALRDAPEEVPSFRLGQHLYGTYRTRLHENNWICIQEDT | protein with |
| | GLLYLNRSLDHSSWEKLSVRNRGFPLLTVYLKVELSPTSLREGECQ | K808X mutation, |
| | WPGCARVYFSFENTSFPACSSLKPRELCFPETRPSFRIRENRPPGT | wherein X is any |
| | FHQFRLLPVQFLCPNISVAYRLLEGEGLPERCAPDSLEVSTRWALD | amino acid other |
| | REQREKYELVAVCTVHAGAREEVVMVPFPVTVYDEDDSAPTFPAGV | than lysine. |
| | DTASAVVEFKRKEDTVVATLRVEDADVVPASGELVRRYTSTLLPGD | |
| | TWAQQTERVEHWPNETSVQANGSFVRATVHDYRLVLNRNLSISENR | |
| | TMQLAVLVNDSDFQGPGAGVLLLHENVSVLPVSLHLPSTYSLSVSR | |
| | RARRFAQIGKVCVENCQAFSGINVQYKLHSSGANCSTLGVVTSAED | |
| | TSGILFVNDTKALRRPKCAELHYMVVATDQQTSRQAQAQLLVTVEG | |
| | SYVAEEAGCPLSCAVSKRRLECEECGGLGSPTGRCEWRQGDGKGIT | |
| | RNFSTCSPSTKTCPDGHCDVVETQDINICPQDCLRGSIVGGHEPGE | |
| | PRGIKAGYGTCNCFPEEEKCFCEPEDIQDPLCDELCRTVIAAAVLF | |
| | SFIVSVLLSAFCIHCYHKFAHKPPISSAEMTERRPAQAFPVSYSSS | |
| | GARRPSLDSMENQVSVDAFKILEDPKWEFPRKNLVLGKTLGEGEFG | |
| | KVVKATAFHLKGRAGYTTVAVKMLKENASPSELRDLLSEFNVLKQV | |
| | NHPHVIKLYGACSQDGPLLLIVEYAXYGSLRGELRESRKVGPGYLG | |
| | SGGSRNSSSLDHPDERALTMGDLISFAWQISQGMQYLAEMKLVHRD | |
| | LAARNILVAEGRKMKISDFGLSRDVYEEDSYVKRSQGRIPVKWMAI | |
| | ESLFDHIYTTQSDVWSFGVLLWEIVTLGGNPYPGIPPERLENLLKT | |
| | GHRMERPDNCSEEMYRLMLQCWKQEPDKRPVFADISKDLEKMMVKR | |
| | RDYLDLAASTPSDSLIYDDGLSEEETPLVDCNNAPLPRALPSTWIE | |
| | NKLYGMSDPNWPGESPVPLTRADGTNTGFPRYPNDSVYANWMLSPS | |
| | AAKLMDTFDS | |
| 7 | MAKATSGAAGLRLLLLLLLPLLGKVALGLYFSRDAYWEKLYVDQAA | Exemplary RET |
| | GTPLLYVHALRDAPEEVPSFRLGQHLYGTYRTRLHENNWICIQEDT | protein with |
| | GLLYLNRSLDHSSWEKLSVRNRGFPLLTVYLKVFLSPTSLREGECQ | Y809X mutation, |
| | WPGCARVYFSFENTSFPACSSLKPRELCFPETRPSFRIRENRPPGT | wherein X is any |
| | FHQFRLLPVQFLCPNISVAYRLLEGEGLPERCAPDSLEVSTRWALD | amino acid other |
| | REQREKYELVAVCTVHAGAREEVVMVPFPVTVYDEDDSAPTEPAGV | than tyrosine. |
| | DTASAVVEFKRKEDTVVATLRVEDADVVPASGELVRRYTSTLLPGD | |
| | TWAQQTERVEHWPNETSVQANGSFVRATVHDYRLVLNRNLSISENR | |
| | TMQLAVLVNDSDFQGPGAGVLLLHENVSVLPVSLHLPSTYSLSVSR | |
| | RARRFAQIGKVCVENCQAFSGINVQYKLHSSGANCSTLGVVTSAED | |
| | TSGILFVNDTKALRRPKCAELHYMVVATDQQTSRQAQAQLLVTVEG | |
| | SYVAEEAGCPLSCAVSKRRLECEECGGLGSPTGRCEWRQGDGKGIT | |
| | RNFSTCSPSTKTCPDGHCDVVETQDINICPQDCLRGSIVGGHEPGE | |
| | PRGIKAGYGTCNCFPEEEKCFCEPEDIQDPLCDELCRTVIAAAVLE | |
| | SFIVSVLLSAFCIHCYHKFAHKPPISSAEMTERRPAQAFPVSYSSS | |
| | GARRPSLDSMENQVSVDAFKILEDPKWEFPRKNLVLGKTLGEGEFG | |
| | KVVKATAFHLKGRAGYTTVAVKMLKENASPSELRDLLSEENVLKQV | |
| | NHPHVIKLYGACSQDGPLLLIVEYAKYGSLRGELRESRKVGPGYLG | |
| | SGGSRNSSSLDHPDERALTMGDLISFAWQISQGMQYLAEMKLVHRD | |
| | LAARNILVAEGRKMKISDFGLSRDVYEEDSYVKRSQGRIPVKWMAI | |
| | ESLFDHIYTTQSDVWSFGVLLWEIVTLGGNPYPGIPPERLENLLKT | |
| | GHRMERPDNCSEEMYRLMLQCWKQEPDKRPVFADISKDLEKMMVKR | |
| | RDYLDLAASTPSDSLIYDDGLSEEETPLVDCNNAPLPRALPSTWIE | |
| | NKLYGMSDPNWPGESPVPLTRADGTNTGFPRYPNDSVYANWMLSPS | |
| | AAKLMDTEDS | |
| 8 | MAKATSGAAGLRLLLLLLLPLLGKVALGLYFSRDAYWEKLYVDQAA | Exemplary RET |
| | GTPLLYVHALRDAPEEVPSFRLGQHLYGTYRTRLHENNWICIQEDT | protein with |
| | GLLYLNRSLDHSSWEKLSVRNRGFPLLTVYLKVELSPTSLREGECQ | G810X mutation, |
| | WPGCARVYFSFENTSFPACSSLKPRELCFPETRPSFRIRENRPPGT | wherein X is any |
| | FHQFRLLPVQFLCPNISVAYRLLEGEGLPFRCAPDSLEVSTRWALD | amino acid other |
| | REQREKYELVAVCTVHAGAREEVVMVPFPVTVYDEDDSAPTFPAGV | than glycine. |
| | DTASAVVEFKRKEDTVVATLRVEDADVVPASGELVRRYTSTLLPGD | |
| | TWAQQTERVEHWPNETSVQANGSFVRATVHDYRLVLNRNLSISENR | |
| | TMQLAVLVNDSDFQGPGAGVLLLHENVSVLPVSLHLPSTYSLSVSR | |
| | RARRFAQIGKVCVENCQAFSGINVQYKLHSSGANCSTLGVVTSAED | |
| | TSGILFVNDTKALRRPKCAELHYMVVATDQQTSRQAQAQLLVTVEG | |
| | SYVAEEAGCPLSCAVSKRRLECEECGGLGSPTGRCEWRQGDGKGIT | |
| | RNFSTCSPSTKTCPDGHCDVVETQDINICPQDCLRGSIVGGHEPGE | |
| | PRGIKAGYGTCNCFPEEEKCFCEPEDIQDPLCDELCRTVIAAAVLE | |
| | SFIVSVLLSAFCIHCYHKFAHKPPISSAEMTERRPAQAFPVSYSSS | |
| | GARRPSLDSMENQVSVDAFKILEDPKWEFPRKNLVLGKTLGEGEFG | |
| | KVVKATAFHLKGRAGYTTVAVKMLKENASPSELRDLLSEENVLKQV | |
| | NHPHVIKLYGACSQDGPLLLIVEYAKYXSLRGELRESRKVGPGYLG | |
| | SGGSRNSSSLDHPDERALTMGDLISFAWQISQGMQYLAEMKLVHRD | |
| | LAARNILVAEGRKMKISDEGLSRDVYEEDSYVKRSQGRIPVKWMAI | |
| | ESLFDHIYTTQSDVWSFGVLLWEIVTLGGNPYPGIPPERLENLLKT | |
| | GHRMERPDNCSEEMYRLMLQCWKQEPDKRPVFADISKDLEKMMVKR | |
| | RDYLDLAASTPSDSLIYDDGLSEEETPLVDCNNAPLPRALPSTWIE | |
| | NKLYGMSDPNWPGESPVPLTRADGTNTGFPRYPNDSVYANWMLSPS | |
| | AAKLMDTFDS | |

### VI. DESCRIPTION OF CERTAIN EMBODIMENTS

As used herein, “TAS0953/HM06” or “HM06/TAS0953” or “HM06” interchangeably refers to 4-amino-N-(4-(methoxymethyl)phenyl)-7-(1-methylcyclopropyl)-6-(3-morpholinoprop-1-yn-1-yl)-7H-pyrrolo[2,3-d]pyrimidine-5-carboxamide in the free base form as well as any salt form thereof, including the di-hydrochloride salt, unless specified otherwise. The di-hydrochloride salt of 4-amino-N-(4-(methoxymethyl)phenyl)-7-(1-methylcyclopropyl)-6-(3-morpholinoprop-1-yn-1-yl)-7H-pyrrolo[2,3-d]pyrimidine-5-carboxamide is also referred to interchangeably as “TAS0953-01/HM06-01” or “HM06-01” or “HM06-01/TAS0953-01.” The molecular formula of the free base form of HM06/TAS0953 is C26H30N6O3 and the molecular weight is 474.57. The structural formula of the free base is:

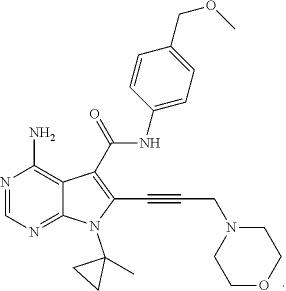 [SMILES: COCc1ccc(NC(=O)c2c(C#CCN3CCOCC3)n(C3(C)CC3)c3ncnc(N)c23)cc1]

The molecular formula of HM06-01/TAS0953-01 is: C26H32O3N6Cl2 and the molecular weight is 547.54. The chemical structure of the di-hydrochloride salt is:

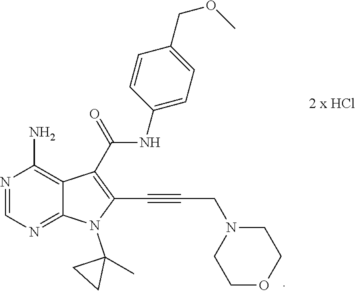 [SMILES: COCc1ccc(NC(=O)c2c(C#CCN3CCOCC3)n(C3(C)CC3)c3ncnc(N)c23)cc1]

In some embodiments, the free base form is the active pharmaceutical ingredient (API). In some embodiments, the di-hydrochloride salt is the active pharmaceutical ingredient (API). In some embodiments, the API is a white to off-white, solid. In some embodiments, the API is freely soluble in water.

A dosage form of a composition comprising HM06/TAS0953 may be any of oral or parenteral forms. The forms of such preparations are not particularly limited, and examples thereof include: oral compositions, such as a tablet, a coated tablet, a pill, a powder, a granule, a capsule, a solution, a suspension, and an emulsions; parenteral compositions, such as an injection, a suppository, and an inhalant; etc. An injection may be intravenously administered alone or as a mixture with a general adjuvant, such as glucose or an amino acid, or as necessary, administered alone intraarterially, intramuscularly, intradermally, subcutaneously, or intraperitoneally. A suppository is intrarectally administered.

In some embodiments, HM06/TAS0953 is prepared as a di-hydrochloride salt (HM06-01/TAS0953-01), formulated in tablets. In some embodiments, the tablets are for oral administration. In some embodiments, the tablets are formulated at a dose of about 10 or about 50 mg/unit (expressed as free base) or another dosage described herein. In some embodiments, the tablets are administered orally as a single tablet or multiple tablets.

In some embodiments, the composition comprises compendial and widely used excipients. In some embodiments, the composition comprises at least one excipient. In some embodiments, the composition comprises at least one antioxidant, at least one filler, at least one disintegrant, at least one surfactant, and/or at least one lubricant. In some embodiments, the composition comprises citric acid, microcrystalline cellulose (e.g., Avicel PH200 LM), lactose (e.g., Lactose 316 Fast Flo), polyvinyl N-pyrrolidone (e.g., Crospovidone), sodium lauryl sulfate, and/or glyceryl behenate (e.g., Compritol ATO 888).

In general, a human patient is administered a composition comprising HM06/TAS0953 at an effective dosage. In some embodiments, a human patient is administered a composition comprising HM06/TAS0953 at a dosage of HM06/TAS0953 equivalent to a range of about 40 mg to about 3000 mg HM06/TAS0953 free base per day. In some embodiments, a human patient is administered a composition comprising HM06/TAS0953 at a dosage of HM06/TAS0953 equivalent to a range of about 40 mg to about 1000 mg HM06/TAS0953 free base per day. In some embodiments, the dosage of HM06/TAS0953 is administered once-daily (QD) or multiple times daily (e.g., BID or TID). In some embodiments, the dosage of HM06/TAS0953 administered is equivalent to a range of about 20 mg to about 1500 mg HM06/TAS0953 free base twice a day (BID). In some embodiments, the dosage of HM06/TAS0953 administered is equivalent to a range of about 20 mg to about 500 mg HM06/TAS0953 free base twice a day (BID).

In some embodiments, the dosage of HM06/TAS0953 administered is equivalent to a range of about 150 mg to about 640 mg HM06/TAS0953 free base per day (e.g., a range of about 75 mg to about 320 mg HM06/TAS0953 free base twice a day). In some embodiments, the dosage of HM06/TAS0953 administered is equivalent to a range of about 160 mg to about 640 mg HM06/TAS0953 free base per day (e.g., a range of about 80 mg to about 320 mg HM06/TAS0953 free base twice a day). In some embodiments, the dosage of HM06/TAS0953 administered is equivalent to a range of about 150 mg to about 500 mg HM06/TAS0953 free base per day (e.g., a range of about 75 mg to about 250 mg HM06/TAS0953 free base twice a day). In some embodiments, the dosage of HM06/TAS0953 administered is equivalent to a range of about 160 mg to about 500 mg HM06/TAS0953 free base per day (e.g., a range of about 80 mg to about 250 mg HM06/TAS0953 free base twice a day). In some embodiments, the dosage of HM06/TAS0953 administered is equivalent to a range of about 320 mg to about 640 mg HM06/TAS0953 free base per day (e.g., a range of about 160 mg to about 320 mg HM06/TAS0953 free base twice a day). In some embodiments, the dosage of HM06/TAS0953 administered is equivalent to a range of about 480 mg to about 640 mg HM06/TAS0953 free base per day (e.g., a range of about 240 mg to about 320 mg HM06/TAS0953 free base twice a day). In some embodiments, the dosage of HM06/TAS0953 administered is equivalent to a range of about 480 mg to about 3000 mg HM06/TAS0953 free base per day (e.g., a range of about 240 mg to about 1500 mg HM06/TAS0953 free base twice a day). In some embodiments, the dosage of HM06/TAS0953 administered is equivalent to a range of about 480 mg to about 2000 mg HM06/TAS0953 free base per day (e.g., a range of about 240 mg to about 1000 mg HM06/TAS0953 free base twice a day). In some embodiments, the dosage of HM06/TAS0953 administered is equivalent to a range of about 480 mg to about 1500 mg HM06/TAS0953 free base per day (e.g., a range of about 240 mg to about 750 mg HM06/TAS0953 free base twice a day). In some embodiments, the dosage of HM06/TAS0953 administered is equivalent to a range of about 480 mg to about 1280 mg HM06/TAS0953 free base per day (e.g., a range of about 240 mg to about 640 mg HM06/TAS0953 free base twice a day). In some embodiments, the dosage of HM06/TAS0953 administered is equivalent to a range of about 480 mg to about 1000 mg HM06/TAS0953 free base per day (e.g., a range of about 240 mg to about 500 mg HM06/TAS0953 free base twice a day).

In some embodiments, the dosage of HM06/TAS0953 administered is equivalent to a range of about 640 mg to about 3000 mg HM06/TAS0953 free base per day (e.g., a range of about 320 mg to about 1500 mg HM06/TAS0953 free base twice a day. In some embodiments, the dosage of HM06/TAS0953 administered is equivalent to a range of about 640 mg to about 2000 mg HM06/TAS0953 free base per day (e.g., a range of about 320 mg to about 1000 mg HM06/TAS0953 free base twice a day. In some embodiments, the dosage of HM06/TAS0953 administered is equivalent to a range of about 640 mg to about 1500 mg HM06/TAS0953 free base per day (e.g., a range of about 320 mg to about 750 mg HM06/TAS0953 free base twice a day. In some embodiments, the dosage of HM06/TAS0953 administered is equivalent to a range of about 640 mg to about 1280 mg HM06/TAS0953 free base per day (e.g., a range of about 320 mg to about 640 mg HM06/TAS0953 free base twice a day. In some embodiments, the dosage of HM06/TAS0953 administered is equivalent to a range of about 640 mg to about 1000 mg HM06/TAS0953 free base per day (e.g., a range of about 320 mg to about 500 mg HM06/TAS0953 free base twice a day).

In some embodiments, the dosage of HM06/TAS0953 (expressed in terms of the free base) includes about 40 mg per day (e.g., about 20 mg twice a day), about 60 mg per day (e.g., about 30 mg twice a day), about 80 mg per day (e.g., about 40 mg twice a day), about 100 mg per day (e.g., about 50 mg twice a day), about 120 mg per day (e.g., about 60 mg twice a day), about 140 mg per day (e.g., about 70 mg twice a day), about 160 mg per day (e.g., about 80 mg twice a day), about 180 mg per day (e.g., about 90 mg twice a day), about 200 mg per day (e.g., about 100 mg twice a day), about 220 mg per day (e.g., about 110 mg twice a day), about 240 mg per day (e.g., about 120 mg twice a day), about 260 mg per day (e.g., about 130 mg twice a day), about 280 mg per day (e.g., about 140 mg twice a day), about 300 mg per day (e.g., about 150 mg twice a day), about 320 mg per day (e.g., about 160 mg twice a day), about 340 mg per day (e.g., about 170 mg twice a day), about 360 mg per day (e.g., about 180 mg twice a day), about 380 mg per day (e.g., about 190 mg twice a day), about 400 mg per day (e.g., about 200 mg twice a day), about 420 mg per day (e.g., about 210 mg twice a day), about 440 mg per day (e.g., about 220 mg twice a day), about 460 mg per day (e.g., about 230 mg twice a day), about 480 mg per day (e.g., about 240 mg twice a day), about 500 mg per day (e.g., about 250 mg twice a day), about 750 mg per day (e.g., about 375 mg twice a day), about 1000 mg per day (e.g., about 500 mg twice a day), about 1280 mg per day (e.g., about 640 mg twice a day), about 1500 mg per day (e.g., about 750 mg twice a day), about 2000 mg per day (e.g., about 1000 mg twice a day), or about 3000 mg per day (e.g., about 1500 mg twice a day).

In some embodiments, the dosage of HM06/TAS0953 (expressed in terms of the free base) is greater than or equal to about 40 mg per day (e.g., about 20 mg twice a day), greater than or equal to about 60 mg per day (e.g., about 30 mg twice a day), greater than or equal to about 80 mg per day (e.g., about 40 mg twice a day), greater than or equal to about 100 mg per day (e.g., about 50 mg twice a day), greater than or equal to about 120 mg per day (e.g., about 60 mg twice a day), greater than or equal to about 140 mg per day (e.g., about 70 mg twice a day), greater than or equal to about 160 mg per day (e.g., about 80 mg twice a day), greater than or equal to about 180 mg per day (e.g., about 90 mg twice a day), greater than or equal to about 200 mg per day (e.g., about 100 mg twice a day), greater than or equal to about 220 mg per day (e.g., about 110 mg twice a day), greater than or equal to about 240 mg per day (e.g., about 120 mg twice a day), greater than or equal to about 260 mg per day (e.g., about 130 mg twice a day), greater than or equal to about 280 mg per day (e.g., about 140 mg twice a day), greater than or equal to about 300 mg per day (e.g., about 150 mg twice a day), greater than or equal to about 320 mg per day (e.g., about 160 mg twice a day), greater than or equal to about 340 mg per day (e.g., about 170 mg twice a day), greater than or equal to about 360 mg per day (e.g., about 180 mg twice a day), greater than or equal to about 380 mg per day (e.g., about 190 mg twice a day), greater than or equal to about 400 mg per day (e.g., about 200 mg twice a day), greater than or equal to about 420 mg per day (e.g., about 210 mg twice a day), greater than or equal to about 440 mg per day (e.g., about 220 mg twice a day), greater than or equal to about 460 mg per day (e.g., about 230 mg twice a day), greater than or equal to about 480 mg per day (e.g., about 240 mg twice a day), greater than or equal to about 500 mg per day (e.g., about 250 mg twice a day), greater than or equal to about 640 mg per day (e.g., about 320 mg twice a day), greater than or equal to about 750 mg per day (e.g., about 375 mg twice a day), greater than or equal to about 1000 mg per day (e.g., about 500 mg twice a day), greater than or equal to about 1280 mg per day (e.g., about 640 mg twice a day), greater than or equal to about 1500 mg per day (e.g., about 750 mg twice a day), greater than or equal to 2000 mg per day (e.g., about 2000 mg twice a day), or greater than or equal to about 3000 mg per day (e.g., about 1500 mg twice a day).

In some embodiments, the dosage of HM06/TAS0953 (expressed in terms of the free base) is greater than or equal to about 640 mg per day (e.g., about 320 mg twice a day). In some embodiments, the dosage of HM06/TAS0953 (expressed in terms of the free base) is greater than or equal to about 1000 mg per day (e.g., about 500 mg twice a day). In some embodiments, the dosage of HM06/TAS0953 (expressed in terms of the free base) is greater than or equal to about 1280 mg per day (e.g., about 640 mg twice a day). In some embodiments, the dosage of HM06/TAS0953 (expressed in terms of the free base) is greater than or equal to about 1500 mg per day (e.g., about 750 mg twice a day). In some embodiments, the dosage of HM06/TAS0953 (expressed in terms of the free base) is greater than or equal to about 2000 mg per day (e.g., about 1000 mg twice a day).

In some embodiments, the dosage of HM06/TAS0953 (expressed in terms of free base) is less than or about 3000 mg per day (e.g., less than or about 1500 mg twice daily). In some embodiments, the dosage of HM06/TAS0953 (expressed in terms of free base) is less than or about 2000 mg per day (e.g., less than or about 1000 mg twice daily). In some embodiments, the dosage of HM06/TAS0953 (expressed in terms of free base) is less than or about 1500 mg per day (e.g., less than or about 750 mg twice daily). In some embodiments, the dosage of HM06/TAS0953 (expressed in terms of free base) is less than or about 1280 mg per day (e.g., less than or about 640 mg twice daily). In some embodiments, the dosage of HM06/TAS0953 (expressed in terms of free base) is less than or about 1000 mg per day (e.g., less than or about 500 mg twice daily). In some embodiments, the dosage of HM06/TAS0953 is less than or about 640 mg per day (e.g., less than or about 320 mg twice daily). In some embodiments, the dosage of HM06/TAS0953 is less than or about 400 mg per day (e.g., less than or about 200 mg twice daily).

In some embodiments, the dosage of HM06/TAS0953 (expressed in terms of free base) is about 150 mg per day (e.g., about 75 mg twice daily). In some embodiments, the dosage of HM06/TAS0953 is about 160 mg per day (e.g., about 80 mg twice daily). In some embodiments, the dosage of HM06/TAS0953 is about 320 mg per day (e.g., about 160 mg twice daily). In some embodiments, the dosage of HM06/TAS0953 is about 640 mg per day (e.g., about 320 mg twice daily). In some embodiments, the dosage of HM06/TAS0953 is about 1000 mg per day (e.g., about 500 mg twice daily). In some embodiments, the dosage of HM06/TAS0953 is about 1280 mg per day (e.g., about 640 mg twice daily). In some embodiments, the dosage of HM06/TAS0953 is about 1500 mg per day (e.g., about 750 mg twice daily). In some embodiments, the dosage of HM06/TAS0953 is about 2000 mg per day (e.g., about 1000 mg twice daily). In some embodiments, the dosage of HM06/TAS0953 is about 3000 mg per day (e.g., about 1500 mg twice daily). In some embodiments, dose modifications may occur during treatment.

In some embodiments, the human patient is 12 years of age or older. In some embodiments, the human patient who is 12 years of age or older weighs less than 50 kg. In some embodiments, the dosage administered is the same for a patient weighing greater than 50 kg and for a patient weighing less than 50 kg.

In some embodiments, for patients weighing less than 50 kg, the dosage of HM06/TAS0953 can be less than or about 3000 mg per day (e.g., 1500 mg twice a day), less than or about 2000 mg per day (e.g., 1000 mg twice a day), less than or about 1500 mg per day (e.g., 750 mg twice a day), less than or about 1280 mg per day (e.g., 640 mg twice a day), less than or about 1000 mg per day (e.g., 500 mg twice a day), less than or about 640 mg per day (e.g., 320 mg twice a day), less than or about 320 mg per day (e.g., 160 mg twice a day), less than or about 160 mg per day (e.g., 80 mg twice a day), less than or about 120 mg per day (e.g., 60 mg twice a day), less than or about 80 mg per day (e.g., 40 mg twice a day), or less than or about 40 mg once daily. For patients weighing more than 50 kg, the dosage of HM06/TAS0953 can be less than or about 3000 mg per day (e.g., 1500 mg twice a day), less than or about 2000 mg per day (e.g., 1000 mg twice a day), less than or about 1500 mg per day (e.g., 750 mg twice a day), less than or about 1280 mg per day (e.g., 640 mg twice a day), less than or about 1000 mg per day (e.g., 500 mg twice a day), less than or about 640 mg once daily (e.g., 320 mg twice a day), less than or about 480 mg per day (e.g., 240 mg twice a day), less than or about 320 mg per day (e.g., 160 mg twice a day), less than or about 240 mg per day (e.g., 120 mg twice a day), less than or about 160 mg per day (e.g., 80 mg twice a day), or less than or about 80 mg per day (e.g., 40 mg twice a day). In some embodiments, dose modifications may occur during treatment.

In some embodiments, the human patient has or has been diagnosed with salivary gland cancer, lung cancer, colorectal cancer, thyroid cancer, breast cancer, pancreatic cancer, ovarian cancer, thyroid cancer, skin cancer, brain cancer. In some embodiments, the human patient has or has been diagnosed with medullary or anaplastic thyroid cancer, metastatic breast cancer, or metastatic pancreatic adenocarcinoma. In some embodiments, the human patient has or has been diagnosed with non-small cell lung cancer (NSCLC). Examples of NSCLCs include adenocarcinomas and large cell carcinomas (non-squamous cell carcinomas), as well as squamous cell carcinomas. In some embodiments, the human patient has or has been diagnosed with locally advanced or metastatic NSCLC.

In some embodiments, a human patient's primary tumor(s) may have metastasized to the central nervous system (CNS). For example, a human patient could have or could have been diagnosed with a primary tumor and CNS metastases. In some embodiments, a human has or has been diagnosed with a metastatic NSCLC with brain and/or leptomeningeal metastases.

As used herein, “brain and/or leptomeningeal metastases” also referred to as brain metastases or leptomeningeal disease, include asymptomatic and symptomatic disease, and can be measurable or not-measurable.

As used herein, and unless specified otherwise, “RET” refers to a gene encoding a RET protein and/or to a RET protein, or a mutation, variation or portion of a RET gene or protein.

As used herein, “RET protein” or “RET polypeptide” refers to a tyrosine kinase receptor encoded by a RET gene (also referred to as a RET proto-oncogene) and may comprise the entirety or a portion of the RET protein.

In some embodiments, a RET protein comprises the amino acid sequence of SEQ ID NO: 2 or a portion thereof.

In some embodiments, a RET protein is a mutated RET protein. In some embodiments, a RET protein comprises the amino acid sequence of SEQ ID NO: 2, SEQ ID NO: 3, SEQ ID NO: 4, SEQ ID NO: 5, SEQ ID NO: 6, SEQ ID NO: 7, or SEQ ID NO: 8, or a portion thereof.

In some embodiments, a RET protein is encoded by a RET gene comprising a RET gene abnormality.

As used herein, “RET gene abnormality” refers to a difference in a mutated RET gene sequence compared to a wild-type RET gene sequence. For example, a RET gene abnormality may be a chromosomal rearrangement, a point mutation, a copy number gain, over expression, and/or ligand-induced activation.

The presence or absence of a RET gene abnormality may be determined by any one of a number of methods understood in the art, including but not limited to, sequencing DNA or RNA or FISH analysis. (E.g., see Subbiah, V et al., Ann Oncol. 2021, 32(2):261-268; Lin J J et al., Ann Oncol. 2020, 31(12):1725-1733; Solomon B J et al., J Thorac Oncol. 2020, 15(4):541-549.) In some embodiments, a RET gene abnormality is detected by sequencing circulating tumor DNA. In some embodiments, a RET gene abnormality is detected by targeted single amplicon sequencing.

In some embodiments, a RET gene comprising a RET gene abnormality encodes a mutated RET protein, such as a RET protein fusion and/or a RET protein comprising a solvent front mutation.

In some embodiments, the cancer or tumor comprises a RET gene abnormality. In some embodiments, the patient has a RET gene abnormality selected from a chromosomal rearrangement (a RET gene fusion), a point mutation, a copy number gain, overexpression, and ligand-induced activation. Overexpression may result from copy number gain or transcriptional upregulation, which may lead to increased local concentration of receptors and abnormal activation. Copy number gain or overexpression may refer to wild-type RET or mutated RET. For example, overexpression of mutant RET have been found in MEN2-associated tumors. Stable overexpression of both mutant M918T and wild-type RET have been found in two SCLC cell lines. Ligand-induced activation of RET may result in stimulation of multiple signal transduction pathways, including the MAP kinase/Erk and PI3 kinase/Akt pathways. This activation may occur for wild-type and mutated RET.

In some embodiments, a RET gene fusion occurs in NSCLC, papillary thyroid carcinoma (PTC), colorectal cancer (CCDC6-RET fusion), or breast cancer (ERC1-RET fusion). In some embodiments, a RET gene point mutation occurs in medullary thyroid carcinoma (MTC), including multiple endocrine neoplasia 2A (MEN2A), MEN2B, or familial medullary thyroid carcinoma (FMTC). In some embodiments, a RET gene copy number gain occurs in NSCLC, breast cancer, pancreatic cancer, or glioblastoma.

In some embodiments, a cancer or tumor comprises a fusion of the RET C terminal kinase domain to N terminal sequences of the kinesin family member 5B (KIF5B-RET), CCDC6-RET (RET-PTC1), NCOA4-RET (RET-PTC3), or TRIM33-RET (RET-PTC7). KIF5B-RET gene fusions may result in a two-fold to 30-fold increased transcription of RET, which suggests that RET kinase activity drives tumorigenesis in these cases. In some embodiments, a RET fusion comprises a fusion of RET with PRKAR1A, TRIM24, GOLGA5, KTN1, MBD1, or TRIM27. In some embodiments, a RET fusion comprises a fusion of RET with TRIM33, KIF5B, CCDC6, or KIF5B.

In some embodiments, the human patient does not have EGFR, KRAS, ALK, HER2, ROS1, BRAF, and/or METex14 activating mutations. RET fusions in NSCLC are in the vast majority of cases mutually exclusive with mutations in EGFR, KRAS, ALK, HER2 and BRAF.

Multi-kinase inhibitors (MKIs) with activity against the RET receptor tyrosine kinase (RTK) include cabozantinib, vandetanib, alectinib, sunitinib, sorafenib, pazopanib, ponatinib, regorafenib, apatinib, sitravatinib, RXDX-105, and lenvatinib. In some embodiments, the human patient has previously been treated with cabozantinib, vandetanib, lenvatinib, RXDX-105, or another multi-kinase inhibitor. In some embodiments, the human patient progressed following the prior treatment. In some embodiments, the human patient developed intolerance to the prior treatment. In some embodiments, the human patient has not previously been treated with a multi-kinase inhibitor.

In some embodiments, the human patient has previously been treated with selpercatinib (LOXO-292), pralsetinib (BLU-667), BOS-172738, or another RET selective inhibitor. In some embodiments, the human patient progressed following the prior treatment. In some embodiments, the human patient developed intolerance to the prior treatment. In some embodiments, the human patient has not previously been treated with a RET selective inhibitor.

Despite clinical improvement seen with targeted agents, patients with RET abnormalities frequently relapse. In some embodiments, the cancer or tumor has or has developed a resistance mutation. In some embodiments, the cancer or tumor is resistant to one or more multi-kinase inhibitors. In some embodiments, the cancer or tumor is resistant to one or more RET selective inhibitors. In some embodiments, the cancer or tumor is resistant to selpercatinib and/or pralsetinib. In some embodiments, the cancer or the tumor comprises cells resistant to selpercatinib and/or pralsetinib.

In some embodiments, HM06/TAS0953 is effective to treat the cancer or tumor that has or has developed a resistance mutation of a RET protein. In some embodiments, the RET gene abnormality comprises a solvent front mutation of a RET protein and/or a mutation in the hinge region of a RET protein. In some embodiments, the RET gene abnormality comprises a solvent front mutation of a RET protein.

In some embodiments, the RET gene abnormality comprises a mutation of a RET protein at amino acid residue 730, 736, 760, 772, 804, 806, 807, 808, 809, 810, and/or 883. In some embodiments, the RET gene abnormality comprises a mutation of a RET protein at amino acid residue 804, 806, 807, 808, 809, and/or 810. In some embodiments, the RET gene abnormality comprises a mutation of a RET protein at amino acid residue 810.

In some embodiments, the RET gene abnormality comprises a mutation of a RET protein comprising a V804X mutation, wherein X is any amino acid other than valine or glutamic acid; b) a Y806X mutation, wherein X is any amino acid other than tyrosine; c) a A807X mutation, wherein X is any amino acid other than alanine; d) a K808X mutation, wherein X is any amino acid other than alanine; e) a Y809X mutation, wherein X is any amino acid other than tyrosine; and/or f) a G810X mutation, wherein X is any amino acid other than glycine.

In some embodiments, the RET gene abnormality comprises a mutation of a RET protein comprising a V804X mutation, wherein X is any amino acid other than valine or glutamic acid. In some embodiments, the RET gene abnormality comprises a mutation of a RET protein comprising a Y806X mutation, wherein X is any amino acid other than tyrosine.

In some embodiments, the RET gene abnormality comprises a mutation of a RET protein comprising a A807X mutation, wherein X is any amino acid other than alanine. In some embodiments, the RET gene abnormality comprises a mutation of a RET protein comprising a K808X mutation, wherein X is any amino acid other than alanine. In some embodiments, the RET gene abnormality comprises a mutation of a RET protein comprising a Y809X mutation, wherein X is any amino acid other than tyrosine. In some embodiments, the RET gene abnormality comprises a mutation of a RET protein comprising a G810X mutation, wherein X is any amino acid other than glycine.

In some embodiments, the RET gene abnormality comprises a mutation of a RET protein comprising: a) a V804L or V804M mutation; b) a Y806C, Y806S, Y806H, or Y806N mutation; and/or c) a G810R, G810S, G810C, G810V, G810D, or G810A mutation. In some embodiments, the RET gene abnormality comprises a V804L or V804M mutation of a RET protein. In some embodiments, the RET gene abnormality comprises a Y806C, Y806S, Y806H, or Y806N mutation of a RET protein. In some embodiments, the RET gene abnormality comprises a G810R, G810S, G810C, G810V, G810D, or G810A mutation of a RET protein. In some embodiments, the RET gene abnormality comprises a G810R mutation of a RET protein.

In some embodiments, the RET gene abnormality comprises a mutation of a RET protein comprising: a) a L730Q or L730R mutation; b) a G736A mutation; c) a L760Q mutation; d) a L772M mutation; and/or e) a A883V mutation. In some embodiments, the RET gene abnormality comprises a L730W or L730R mutation of a RET protein. In some embodiments, the RET gene abnormality comprises a G736A mutation of a RET protein. In some embodiments, the RET gene abnormality comprises a L760Q mutation of a RET protein. In some embodiments, the RET gene abnormality comprises a L772M mutation of a RET protein. In some embodiments, the RET gene abnormality comprises a A883V mutation of a RET protein.

### VII. EXAMPLES

### Example 1. Brain Migration Property of HM06/TAS0953

In an initial study, the ability of HM06/TAS0953 to migrate across the blood brain barrier was evaluated. HM06/TAS0953 was dissolved in 0.5% HPMC, 0.1N hydrochloric acid and orally administered in a single dose to BALB/cAJcl-nu/nu mice (CLEA Japan, Inc.) that were subcutaneously implanted with the BaF3/KIF5B-RET_RFP cell line (i.e. a cell line harboring a KIF5B RET fusion). One hour after administration, blood was collected from the inferior vena cava under isoflurane anesthesia, and then the whole brain was removed. The blood sample was centrifuged to obtain a plasma sample. The brain was homogenized with a 3-fold amount of water using an ultrasonic homogenizer to obtain a brain homogenate.

Compound concentration of HM06/TAS0953 in the plasma and in the brain homogenate was measured by LC-MS/MS, and the compound concentration in the brain homogenate was multiplied by a factor of 4 to calculate the compound concentration in the brain. See Table 2, below. A Kp value (=compound concentration in brain homogenate/compound concentration in plasma) was calculated from the ratio of the compound concentrations in the brain/plasma. Non-binding compound concentrations in the plasma and in the brain were calculated from the plasma protein non-binding ratio and brain protein non-binding ratio and a Kp,uu value was calculated from the ratio of the non-binding compound concentrations in the brain/plasma. A brain migration property was evaluated on the basis of the calculated Kp value and Kp,uu value. A compound showing a Kp value of 0.1 or more was considered to have a brain migration property, and a compound showing a Kp,uu value of 0.3 or more was considered to have an excellent brain migration property (Varadharajan S, et al. J Pharm Sci. 2015, 104:1197-1206).

TABLE 2
Brain Penetration

| | | | | | Unbound | | |
|---|---|---|---|---|---|---|---|
| | | Concentration | | | Concentration | | |
| Time | Mouse | (μM or nmol/g) | | | (μM or nmol/g) | | |
| (hr) | # | Plasma | brain | K_{p,brain} | plasma | brain | K_{p,uu,brain} |
| 1 | 1 | 29.3 | 55.0 | 1.88 | 2.16 | 4.29 | 1.98 |
| | 2 | 19.6 | 29.9 | 1.53 | 1.45 | 2.33 | 1.61 |
| | mean | | | 1.71 | | | 1.80 |

K_{p,brain} = Brain-to-plasma concentration ratio (C_{brain}/Cₚₗₐₛₘₐ)
K_{p,uu,brain} = Unbound brain-to-plasma concentration ratio ((C_{brain} × f_{u,brain})/(Cₚₗₐₛₘₐ × f_{u,plasma})
f_{u,plasma} (mouse): 0.074
f_{u,brain} (mouse): 0.078

HM06/TAS0953 exhibited a high Kp value and a high Kp,uu value, indicative of an excellent brain migration property. This data suggests that HM06/TAS0953 may be effective for treating a brain metastatic lesion or other CNS diseases.

### Example 2. HM06/TAS0953 has Potent Efficacy in Brain Metastasis Model

The efficacy of HM06/TAS0953 in treating tumors harboring a RET abnormality was tested in a brain metastasis model harboring a KIF5B-RET fusion. HM06/TAS0953 showed potent and stable effect.

In this model, NIH3T3 cells harboring a KIF5B-RET Luc fusion (2.5×10⁵ cells/mouse) were implanted to brain of athymic nude mice (Charles River Laboratories Japan) at a depth of 3 mm at a position 3-mm anterior and 2-mm right lateral to the lambda. Six days after implantation, mice were intravenously injected with luciferin (FUJIFILM Wako Pure Chemical Corporation) and the luminescence of the mice was measured with an In Vivo Imaging System (Lumina II, PerkinElmer). The total flux (p/s) in the region of interest of the back and side of the mouse were quantified using Living Image software (PerkinElmer), respectively, and the total photons obtained by adding both of the total flux values were utilized as the luminescence signal. Mice were randomly allocated into three groups of ten mice to equalize the mean total photons in each group, and then orally administered vehicle (0.5% hydroxypropyl methylcellulose containing 0.1 mol/L hydrochloric acid) or TAS0953 (12.5 mg/kg or 50 mg/kg) twice a day (b.i.d) from day 7. The luminescence signals of the mice were measured once a week until the end of the study.

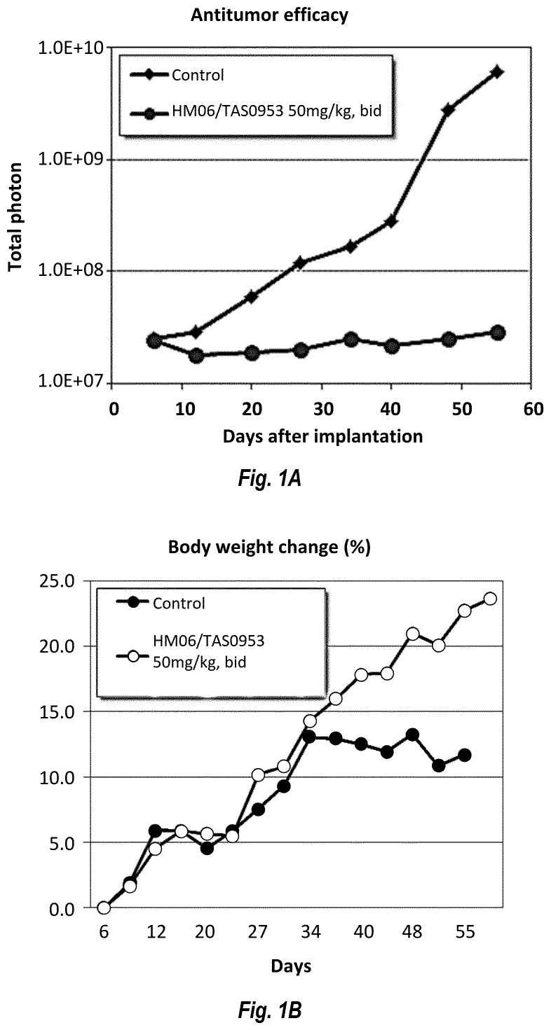

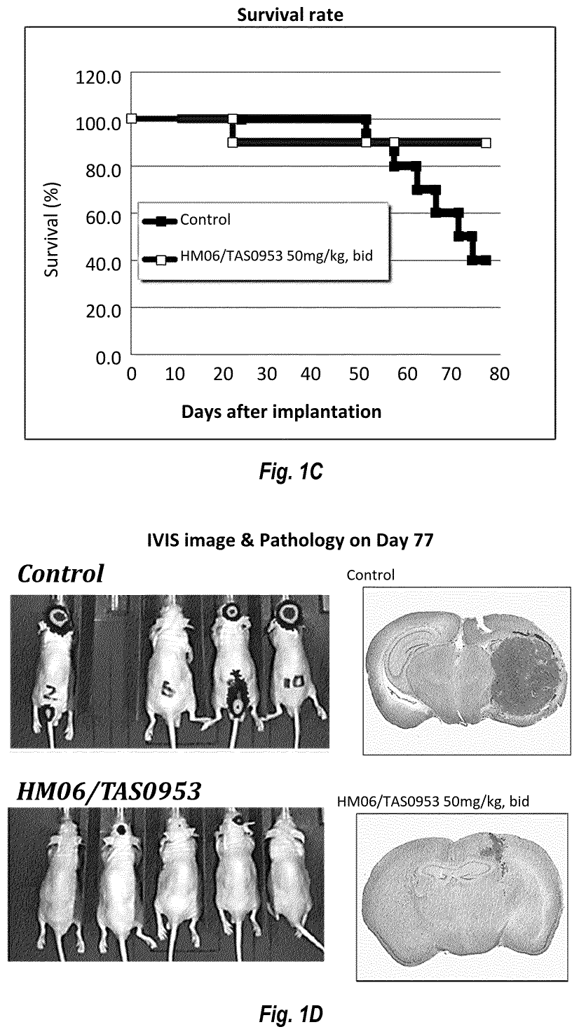

HM06/TAS0953 showed potent and stable effect. FIG. 1A shows antitumor efficacy of HM06/TAS0953 compared to vehicle control when administered at 50 mg/kg BID. FIG. 1B shows percent body weight change and that mice treated with HM06/TAS0953 tolerated the compound well at the dose tested whereas in the vehicle control group, animal death by tumor burden and body weight gain suppression were observed. FIG. 1C shows a higher survival rate in the HM06/TAS0953 group than the vehicle control group. FIG. 1D shows an in vivo imaging system (IVIS) image and pathology in the brains of treated mice compared to vehicle control.

These data establish that HM06/TAS0953 could have a potent antitumor effect in brain metastasis in patients harboring RET abnormalities and indicates a prolonged survival period. These data also show HM06/TAS0953 has good brain penetrability in mice.

### Example 3. HM06/TAS0953 has Efficacy in Vandetanib-Refractory Tumor Model

The effects of HM06/TAS0953 after vandetanib treatment were assessed as compared to continuous vandetanib treatment to evaluate whether switching to HM06/TAS0953 would have a significant effect on the tumor size. The results suggest that HM06/TAS0953 would be effective on refractory tumors.

A mouse fibroblast cell line transfected with fusion kinase KIF5B-RET (NIH3T3 KIF5B-RET) was transplanted at 5×10⁶ cells/mouse into the right chest of 6-week old male BALB/cA Jcl-nu mice. After the tumor transplant, the major axis (mm) and the minor axis (mm) of the tumor were measured using a caliper to calculate the tumor volume (TV) according to the following formula (A). Then, the mice were allocated to various groups so that the average TV's of the various groups would be uniform. The day on which this grouping (n=5-6/group) was performed was designated Day 1.

Vandetanib was orally administered at 100 mg/kg/day every day until the timing that the mean tumor volume in the vandetanib-treated mice exceeded three times bigger than the mean tumor volume on Day 1. On Day 16, the vandetanib treatment groups were divided into two groups: Group 1: Continuous vandetanib treatment group; Group 2: HM06/TAS0953 treatment group. In the HM06/TAS0953 treatment group, HM06/TAS0953 was orally administered at 100 mg/kg/day (50 mg/kg, BID) every day after switching from vandetanib to HM06/TAS0953.

As an index of the antitumor effect, the TV on Day 31 was measured for each group, and the relative tumor volume (RTV) for Day 1 was calculated by the following formula (B) to evaluate the antitumor effect.

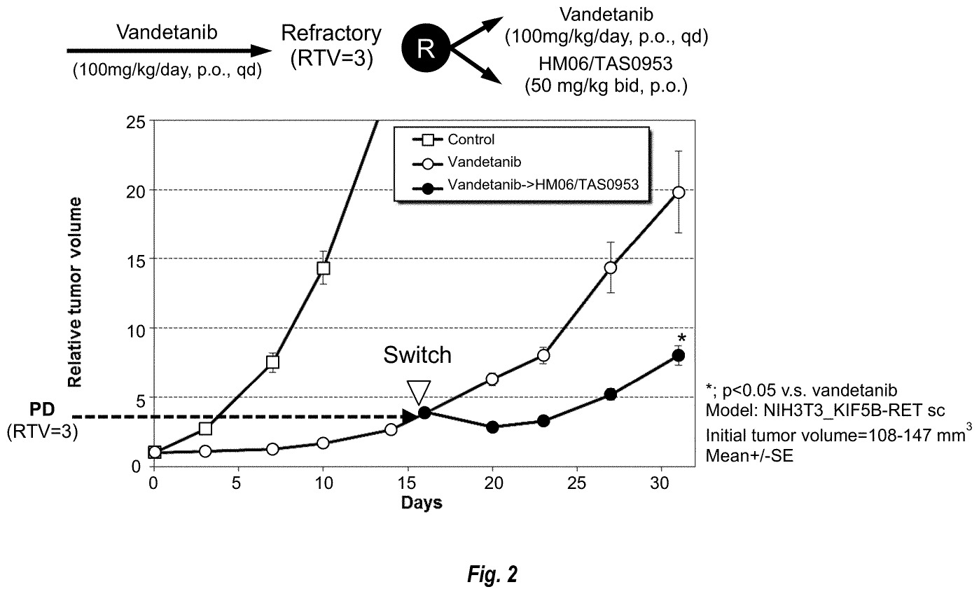

The results are shown in FIG. 2, with the symbol*indicating a statistical difference between the HM06/TAS0953 treatment group compared to the continuous vandetanib treatment group.
(A): TV (mm³)=(Major axis×minor axis²)/2
(B): RTV=(TV on Day n)/(TV on Day 1)
n: indicated measurement day

Statistically, the mean RTV value of an HM06/TAS0953 treatment group is significantly (Student t-test, p<0.05) smaller than that of a continuous vandetanib treatment group. These data show that HM06/TAS0953 was effective on a vandetanib-refractory tumor.

### Example 4. Preclinical Evaluation of HM06/TAS0953 in Lung Cancer Cell Lines Driven by RET Rearrangements

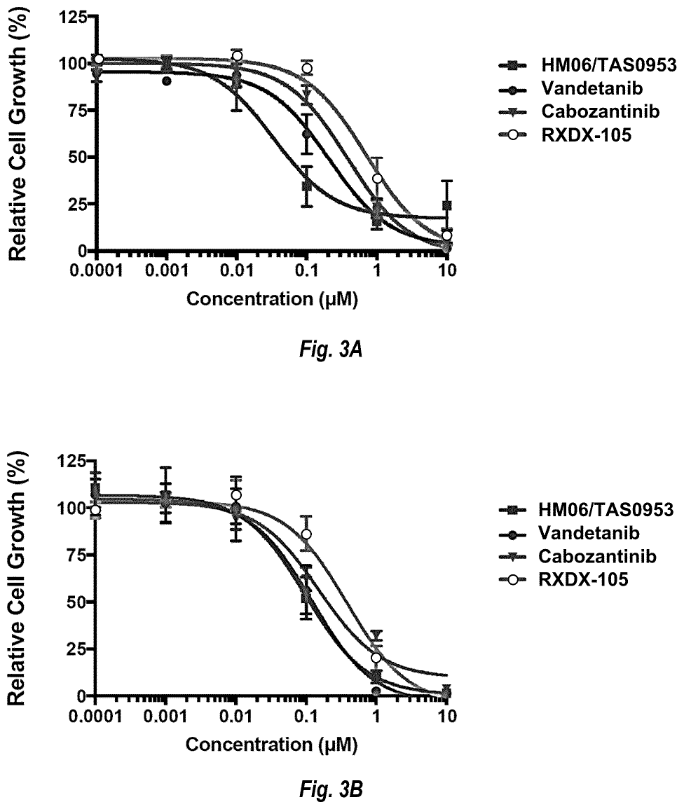

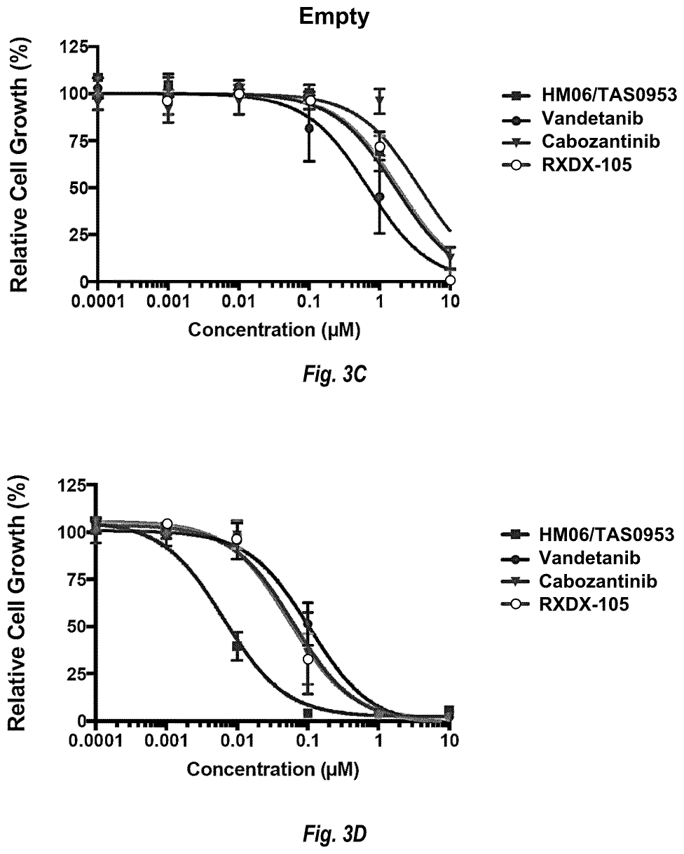

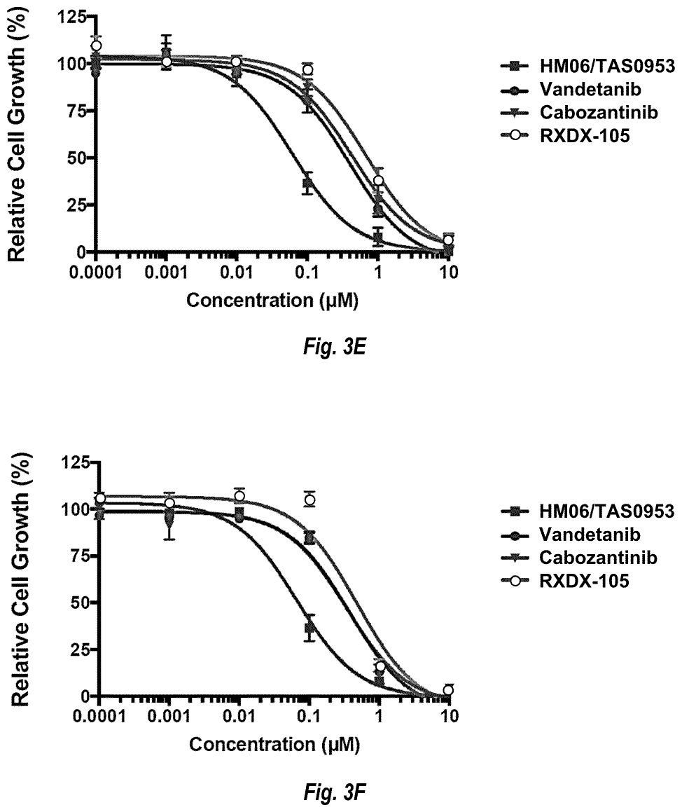

The efficacy of HM06/TAS0953 at inhibiting growth of RET-fusion positive cell lines in comparison with three RET multi-kinase inhibitors (cabozantinib, RXDX-105, and vandetanib) was then examined. The efficacy of HM06/TAS0953 was examined in two cell lines derived from treatment-naïve samples. One of the cell lines was derived from a sample obtained from a patient who was never treated with any anti-cancer therapy, and which harbors a KIF5B-RET fusion. Treatment with HM06/TAS0953 inhibited growth of cells with IC50=0.03 μM (FIG. 3A). This was at 7-24-fold more potent than the multi-kinase inhibitors cabozantinib, RXDX-105 and vandetanib.

HM06/TAS0953 was also tested in an isogenic pair of cell lines to examine efficacy against a CCDC6-RET fusion-driven cell line and non-specific effects in a nontumorigenic control cell line. HM06/TAS0953 inhibited growth of the CCDC6-RET fusion cell line with IC50=0.1 μM (FIG. 3B). The isogenic counterpart of this cell line that expresses the empty control plasmid was much less sensitive to HM06/TAS0953 (17-fold less) (FIG. 3C). The CCDC6-RET fusion-cell lines were equally sensitive to cabozantinib, RXDX-105 and vandetanib.

HM06/TAS0953 was tested in a cell line that harbors a TRIM33-RET fusion. Growth of cells was inhibited by HM06/TAS0953 with IC50=0.006 μM, which was 8-20-fold lower than the IC50 for inhibition of growth by the 3 multi-kinase RET inhibitors (FIG. 3D).

The inhibition effects of HM06/TAS0953 were examined in RET fusion-positive cell lines that are resistant to RET multi-kinase inhibitors. HM06/TAS0953 inhibited growth of a cell line derived from a sample from a patient who was resistant to cabozantinib (CCDC6-RET fusion) with IC50=0.06 μM. This was 11.5-fold lower than the IC50 for inhibition of growth of these cells by cabozantinib. HM06/TAS0953 was also more potent at inhibiting growth of the cabozantinib-resistant cells compared to RXDX-105 and vandetanib (FIG. 3E).

HM06/TAS0953 inhibited growth of a cell line derived from a sample obtained from a patient who acquired resistance to RXDX-105 (KIF5B-RET fusion) with IC50=0.07 μM (FIG. 3F). This was significantly lower than the IC50 for inhibition of growth by cabozantinib (0.34 μM, 95% CI: 0.21-0.54), RXDX-105 (0.49 μM, 95% CI: 0.31-0.76), or vandetanib (0.38 μM, 95% CI: 0.29-0.49).

The results suggest that HM06/TAS0953 is more effective than other RET multi-kinase inhibitors at inhibiting growth of cell lines with RET fusions. The results support HM06/TAS0953 as being effective against cell lines that are refractory to the inhibitory action of RET multi-kinase inhibitors. In addition, HM06/TAS0953 was effective against cell lines with RET fusions consisting of three different N-terminal fusion partners (CCDC6, KIF5B and TRIM33).

### Example 5. Evaluation of HM06/TAS0953 Efficacy in Preclinical Animal Models of Lung Cancer Driven by RET Rearrangements

The efficacy of HM06/TAS0953 at reducing growth of xenograft tumors was confirmed and efficacy compared to vandetanib, a known multi-kinase inhibitor with anti-RET activity, was further examined. Efficacy was observed in preclinical animal models of RET fusion-positive cancers that are sensitive to RET inhibitors by implanting either isogenic (NIH-3T3-CCDC6-RET) or patient-derived cells (TRIM33-RET fusion) into immune compromised mice to generate xenograft tumors (Examples 5A and 5B). In addition, efficacy was observed in several patient derived xenograft (PDX) models developed from samples from patients who had developed resistance to either RXDX-105 or cabozantinib (Example 5C). As set forth in detail below, in all models, HM06/TAS0953 was either superior to, or as effective as vandetanib at inhibiting tumor growth, and in some cases, inducing tumor regression. Treatment of mice bearing RET-dependent xenograft tumors with HM06/TAS0953 resulted in a significant reduction in tumor growth with as little as 12.5 mg/kg BID dose. At doses of 50 mg/kg BID or 100 mg/kg QD there were significant reductions in tumor growth, including approximately 100% regression of TRIM33-RET fusion xenograft tumors. Similarly, treatment with 50 mg/kg BID or 100 mg/kg QD HM06/TAS0953 significantly reduced growth of cabozantinib-resistant PDX tumors, resulting in approximately 50% tumor regression. HM06/TAS0953 treatment also caused a significant reduction in the growth of two PDX models derived from RXDX-105-resistant tumors. Mice treated with vandetanib lost a significant amount of weight whereas mice treated with HM06/TAS0953 tolerated the compound well at all doses tested in this study.

The effect of HM06/TAS0953 on growth of xenograft tumors was further observed in a preclinical brain orthotopic model (TRIM33-RET fusion) (Example 5D). Treatment with 50 mg/kg BID HM06/TAS0953 completely blocked growth of tumors in the brain and resulted in a significant increase in survival of tumor-bearing mice. The results suggest that HM06/TAS0953 is an effective anti-RET inhibitor that can block growth of RET fusion-positive xenograft tumors implanted the subcutaneous flanks or in the brain of mice.

Methods: Six-week-old female NSG (NOD/SCID gamma) mice (Envigo, Madison, WI) were used for generation of PDX models and for all efficacy studies except for NIH-3T3 xenograft studies where athymic Nude mice (Envigo, Madison, WI) were used. Mice were randomly assigned into groups of 5 mice after a robust signal was detected and treatment was initiated 22 days after implantation of cells with vehicle or 50 mg/kg HM06/TAS0953 BID. Luciferase signal was recorded weekly and animals were weighed twice weekly. Mice were sacrificed when signs of sickness such as a lack of coordination or excessive weight loss and fatigue were detected. In the treatment group, one animal was found dead early in the study and therefore the treatment group shrank to 4 animals only.

Vehicles and compounds: Cabozantinib was mixed in 30% propylene glycol, 5% Tween 80, 65% D5W (dextrose 5% in water) to generate a suspension. Vandetanib was mixed in 1% sodium carboxymethyl cellulose (CMC-Na) to generate a suspension. HM06/TAS0953 was mixed in 0.1 N HCl and 0.5% hypromellose (HPMC) to generate a suspension. RXDX-105 was mixed in 15% captisol to generate a suspension. The NIH-3T3 model, TRIM33-RET fusion model, PDX model obtained from a patient sample obtained after resistance to RXDX-105 (CCDC6-RET), and the model obtained from a patient who was resistant to cabozantinib (CCDC6-RET) were treated with HM06/TAS0953 free base. All other models were treated with HM06 di-hydrochloride salt form.

Statistical Analysis: Data sets were compared by Two-way Anova, with Tukey's or Sidaks' multiple comparison tests to determine significance. P<0.05 was considered a statistically significant difference between two values or data sets. Survival curves were compared using the Log-rank (Mantel-Cox) test. 95% confidence intervals and all statistical analysis were conducted using Graphpad Prism v7 software.

### Example 5A: HM06/TAS0953 is Effective at Inhibiting Growth of RET Inhibitor-Sensitive Xenograft Tumors

The ability of HM06/TAS0953 to inhibit growth of xenograft tumors that were derived from cells that were never treated with a RET inhibitor was examined. NIH-3T3 cells stably expressing a CCDC6-RET fusion protein were implanted into the subcutaneous flank of athymic Nude mice. When tumors reached approximately 100 mm³, mice were randomly assigned to groups of 5 animals and treatment initiated (day 6). The NIH-3T3-CCDC6-RET cell line was generated by stable expression of a CCDC6-RET fusion cDNA. Athymic Nude immune deficient mice bearing NIH-3T3-CCDC6-RET xenograft tumors were treated with 12.5-100 mg/kg HM06/TAS0953 either once daily (QD) or twice daily (BID). Vandetanib (100 mg/kg QD) was used for comparison.

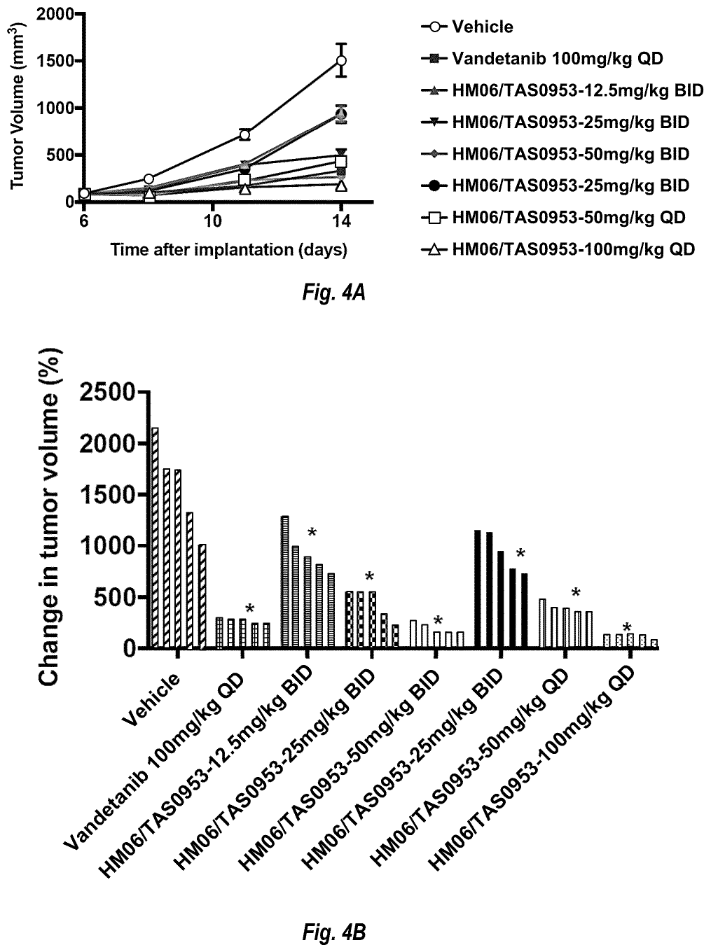

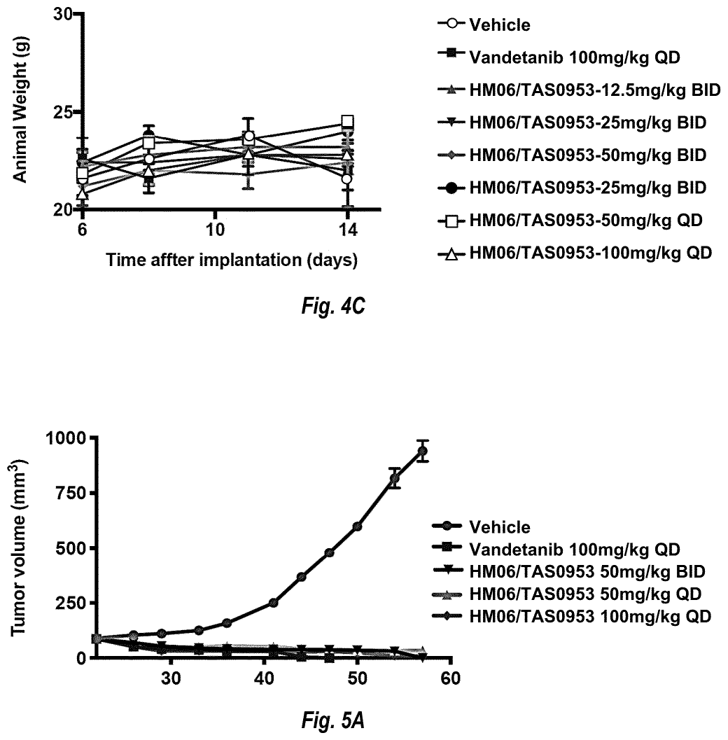

Treatment with HM06/TAS0953 resulted in a significant reduction in tumor growth at all doses tested (FIGS. 4A and 4B). Vandetanib treatment also resulted in a significant reduction in tumor volume compared to the vehicle-treated group. However, HM06/TAS0953 was more effective than vandetanib when administered at 50 mg/kg BID or 100 mg/kg QD. In these experiments a much higher dose of vandetanib (100 mg/kg) than has been shown to inhibit RET fusion-dependent tumor growth (50 mg/kg, QD) was used. (Suzuki M et al., Cancer Sci 0.2013, 104(7):896-903 doi 10.1111/cas.12175.) These results suggest that HM06/TAS0953 is more effective that vandetanib at reducing growth of RET fusion-driven tumors. There was no significant reduction in animal weight at any of the doses of HM06/TAS0953 used (FIG. 4C).

### Example 5B: HM06/TAS0953 is Effective at Inhibiting Growth of Patient-Derived Cell Line Xenografts Driven by RET Fusion

The efficacy studies were extended to a patient-derived cell line xenograft model. Xenograft tumors harboring a TRIM33-RET fusion were implanted into the subcutaneous flank of NSG mice. When tumors reached approximately 100 mm³, mice were randomly assigned to groups of 5 animals and treatment initiated (day 22). (Somwar R et al., J Clin Oncol. 2016, 34(15_suppl):9068.)

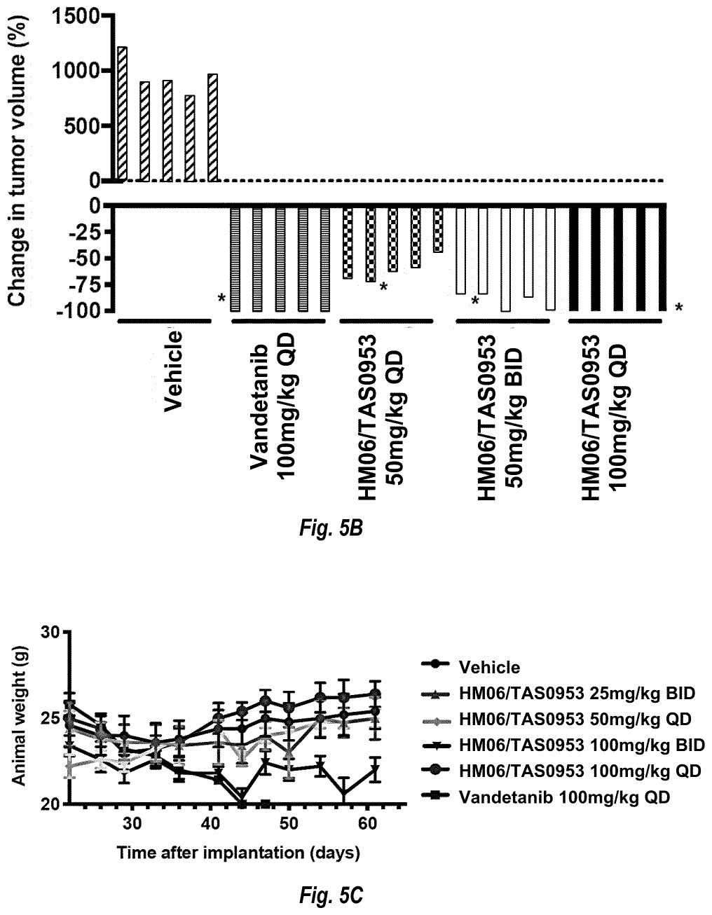

Treatment with HM06/TAS0953 caused a significant reduction in tumor growth at the 3 different doses tried (FIG. 5A). In all groups each xenograft tumor regressed. Tumors shrank by 60.7±5% when treated with 50 mg/kg HM06/TAS0953 once daily (FIG. 5B). No palpable tumors remained on mice treated with 50 mg/kg BID and there was a 90±4% reduction in tumor growth when animals were treated with 100 mg/kg QD HM06/TAS0953 (FIG. 5B). There was no significant change in animal weight when comparing the starting and ending animal weight in any of the HM06-treated groups. Although treatment with vandetanib resulted in complete tumor regression, animals lost a significant amount of weight, and by day 47 all animals in this group had to be sacrificed (FIG. 5C). Each animal in the vandetanib-treated group started losing weight by the third day of treatment initiation.

### Example 5C: HM06/TAS0953 is Effective at Inhibiting Growth of PDX Tumors that are Refractory to RET Multi-Kinase Inhibitors

To further explore the effectiveness of HM06/TAS0953 to inhibit tumor growth, the efficacy of the inhibitor on growth of tumors in three PDX models that were resistant to RXDX-105 and one that was resistant to cabozantinib was examined.

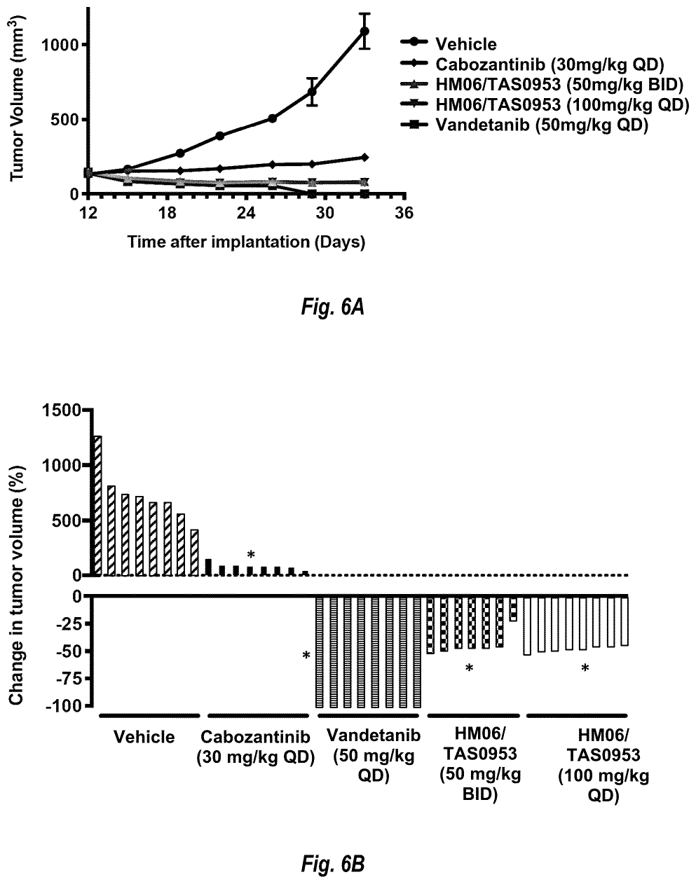

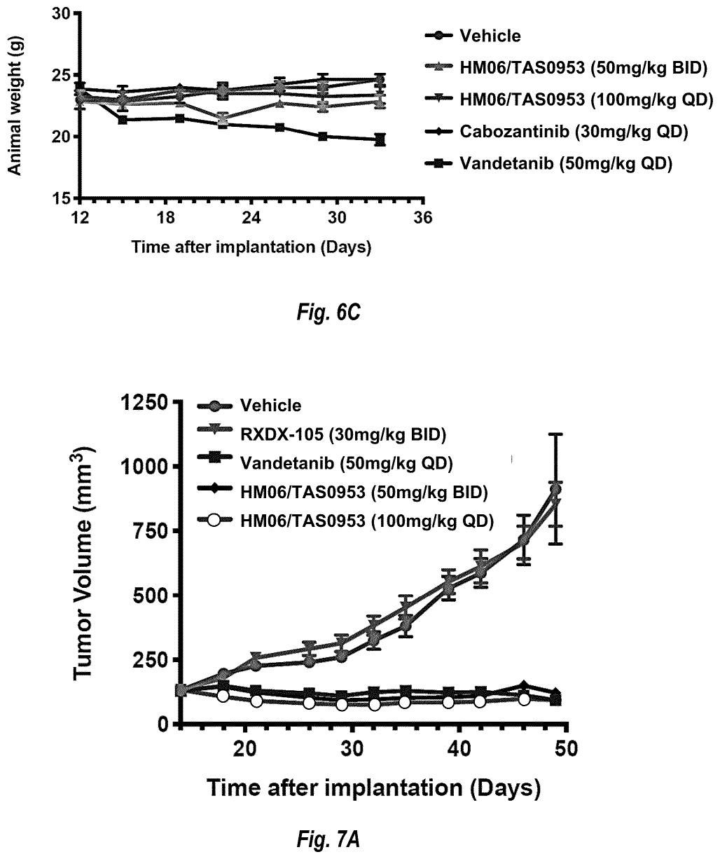

PDX tumors derived from tumor samples obtained from a patient who was no longer responding to cabozantinib (CCDC6-RET) were minced, mixed with Matrigel, and then implanted into the subcutaneous flank of NSG mice. When tumors reached approximately 100 mm³, mice were randomly assigned to groups of 8 animals and treatment initiated (day 12). Treatment of tumor bearing mice with vandetanib caused a significant reduction in tumor growth (FIG. 6A) with no palpable tumors remaining at the end of the study (FIG. 6B). All tumors in the group shrank by 100%. However, the animals lost a significant amount of weight (FIG. 6C). Although cabozantinib (30 mg/kg QD) treatment reduced tumor growth significantly when compared to vehicle-treated group (p<0.05), there was no tumor shrinkage with all tumors in the group showing some growth (FIG. 6B). Treatment with 50 mg/kg BID or 100 mg/kg QD HM06/TAS0953 caused significant reductions in tumor growth (FIG. 6A), with tumors shrinking by 43.7±3.8% and 47.7±0.9%, respectively. One animal was found dead in the HM06-50 mg/kg BID group on day 25 and therefore, there were only 7 animals in this group at the end of the experiment. The cause of death of the animal was unclear.

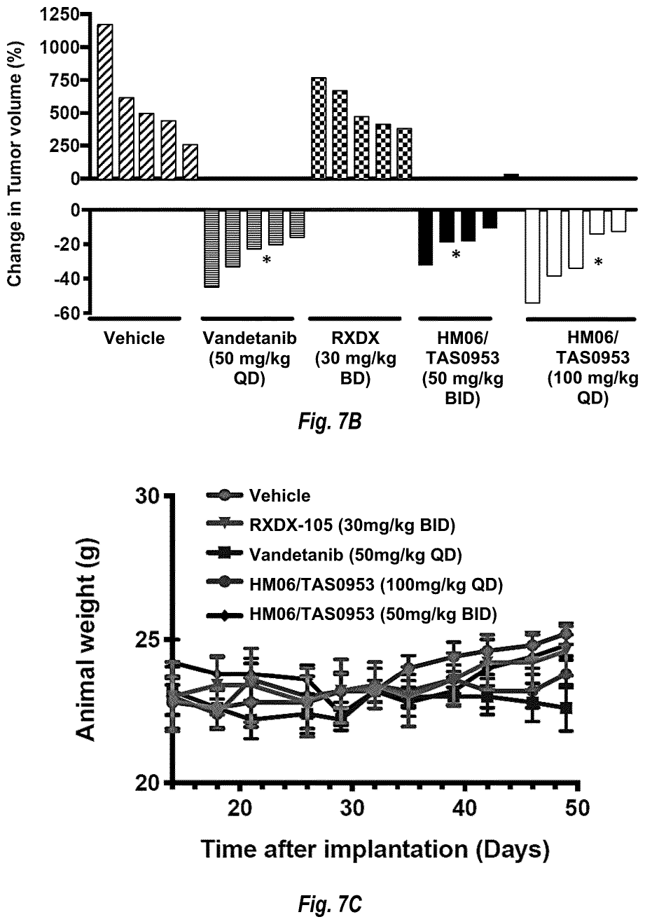

A PDX tumor derived from a patient who was resistant to RXDX-105 therapy at the time of collection of tumor samples (CCDC6-RET), were minced, mixed with Matrigel, and then implanted into the subcutaneous flank of NSG mice. When tumors reached approximately 100 mm³, mice were randomly assigned to groups of 5 animals and treatment initiated (day 14). Treatment with RXDX-105 (30 mg/kg BID) did not cause a significant change in tumor volume (p>0.05) compared to the vehicle treated group, demonstrating that the model was resistant to RXDX-105 (FIG. 7A). Treatment with vandetanib (50 mg/kg QD) caused a significant reduction in tumor volume with tumors shrinking by 27.2±5.2% (p<0.05) (FIGS. 7A and 7B). For this study and all subsequent studies, 50 mg/kg QD vandetanib was used due to the significant loss of weight observed in FIG. 5C. Similarly, treatment with HM06/TAS0953 (50 mg/kg BID or 100 mg/kg, QD) caused a significant reduction in tumor volume compared to vehicle-treated tumors, with tumors shrinking by 8.6±11.8% and 30±7.7%, respectively (FIGS. 7A and 7B). Treatment with either dose of HM06/TAS0953 did not affect animal weight in the groups significantly (p>0.05) (FIG. 7C).

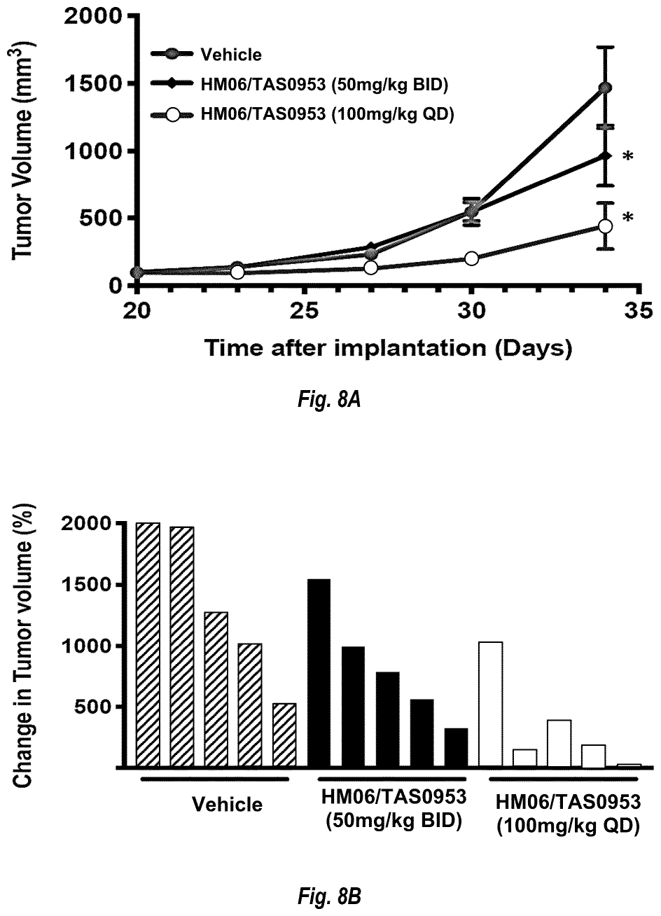

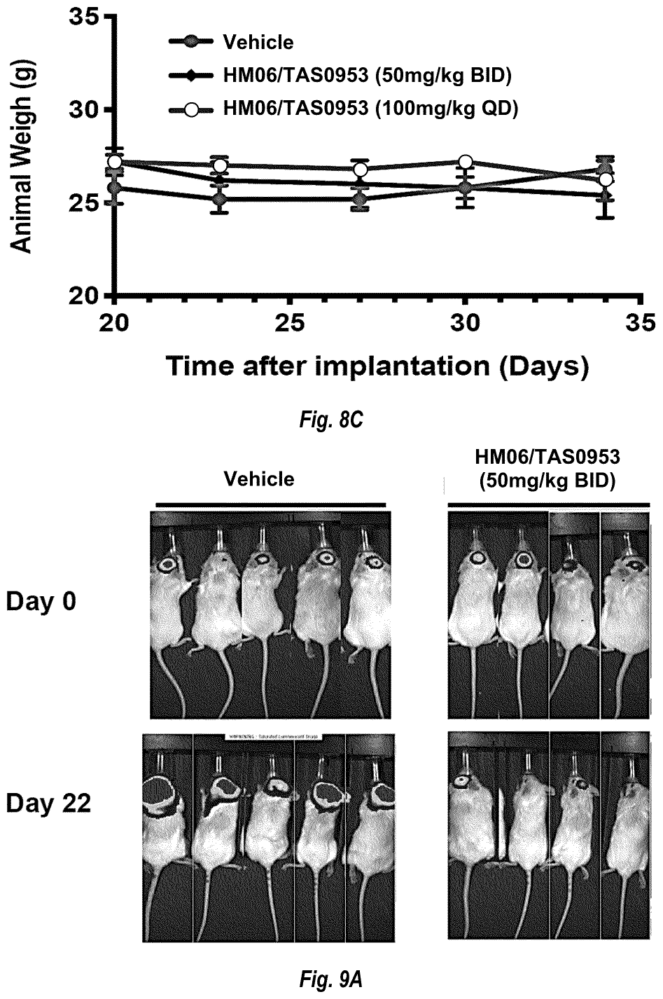

PDX tumors derived from a patient who had a poor response to RXDX-105 (CCDC6-RET), were minced, mixed with matrigel, and then implanted into the subcutaneous flank of NSG mice. When tumors reached approximately 100 mm³, mice were randomly assigned to groups of 8 animals and treatment initiated (day 12). The efficacy of 50 mg/kg BID and 100 mg/kg QD HM06/TAS0953 was tested in this model. Treatment with HM06/TAS0953 at a dose of 50 mg/kg BID caused a small but significant reduction in tumor volume compared to vehicle-treated group (FIG. 8A). Treatment with HM06/TAS0953 at a dose of 100 mg/kg QD was more effective at slowing tumor growth. As shown in FIG. 8A, there was no tumor shrinkage (FIG. 8B). HM06/TAS0953 did not cause any significant change in weight of animals in any groups (FIG. 8C).

These results suggest that HM06/TAS0953 is effective at reducing growth of PDX tumors that were refractory to cabozantinib and RXDX-105.

### Example 5D: HM06/TAS0953 is Effective at Inhibiting Growth of a Brain Orthotopic Xenograft Tumor Model with a RET Fusion

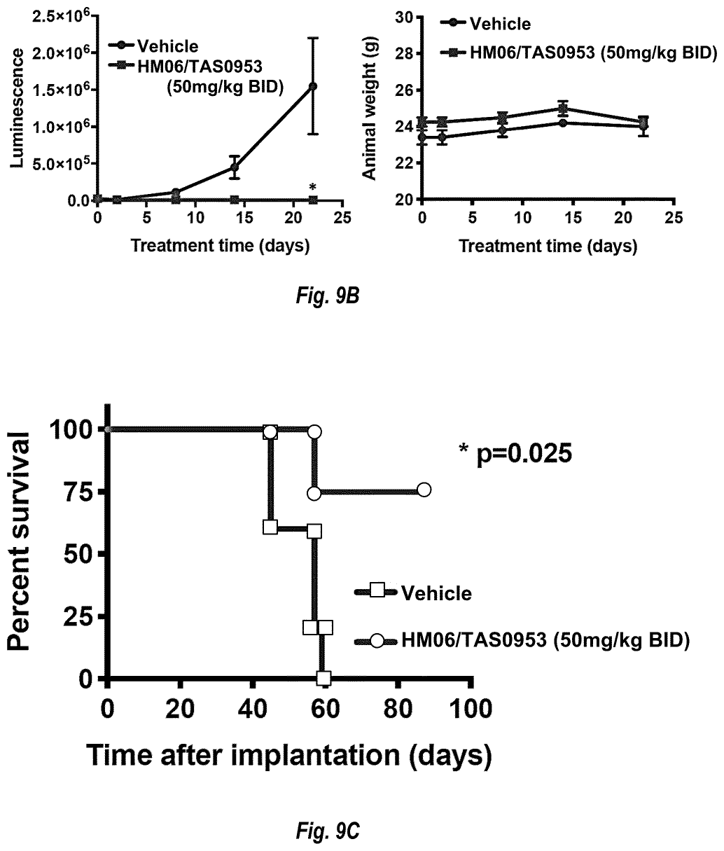

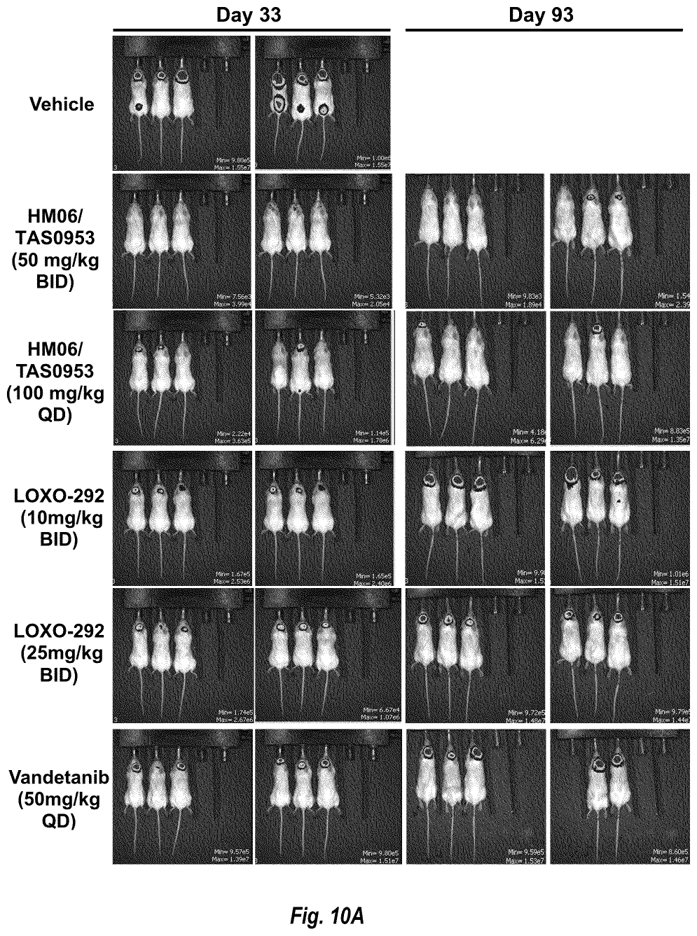

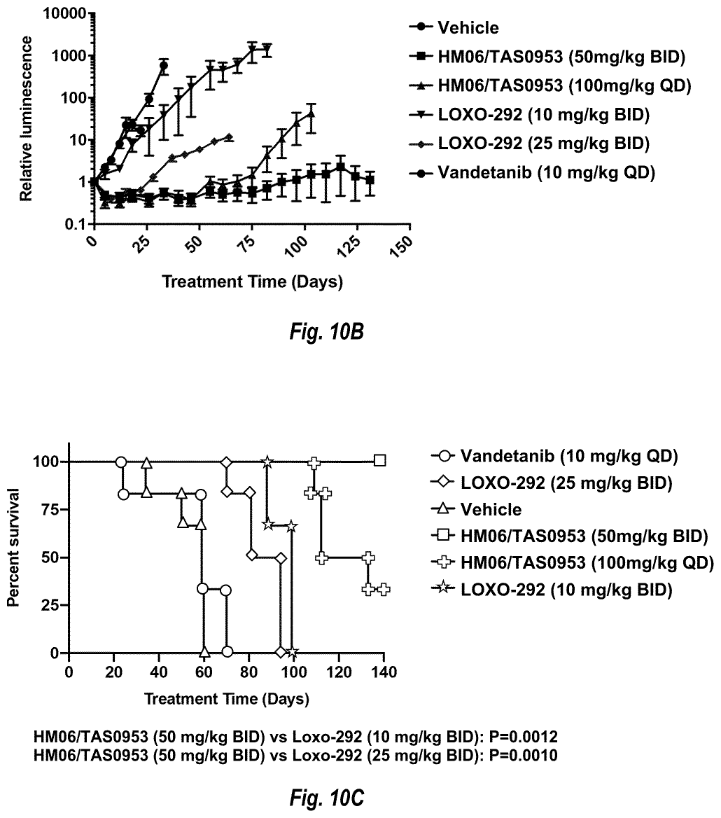

To assess HM06's effect against RET fusion-positive tumors that reside in the brain, xenograft tumors harboring a TRIM33-RET fusion were implanted into the brain of NSG mice. These cells were engineered to express firefly luciferase in order to facilitate bioluminescence imaging in vivo. Xenograft tumors were digested, and then single cells implanted into the brain of NSG mice. Bioluminescence imaging was conducted weekly and when a robust signal was detected, treatment began 22 days after implantation of tumor cells (day 0 on panels A and B). Mice were imaged weekly and after a robust signal was detected treatment was initiated (22 days after implantation) with 50 mg/kg HM06/TAS0953 BID or vehicle. The bioluminescence signals were quantitated as described in the Materials and Methods section. Vehicle-treated mice rapidly developed tumors as can be seen by the strong bioluminescence signal (FIGS. 9A and 9B) over the average starting signal (p<0.05). However, by day 22 there was a significant difference between the vehicle-treated group and the HM06-treated group (p<0.05). HM06-treated mice survived significantly longer than vehicle-treated mice (p<0.05) with three mice still surviving at the end of the experiment, 28 days after the last animal from the vehicle-treated group had to be sacrificed due to tumor burden (FIG. 9C). HM06/TAS0953 treatment did not have any adverse effect on the weight of the experimental animals (FIG. 9B, right panel). These results confirm that HM06/TAS0953 crosses into the brain and is effective against RET-rearranged tumors that reside there.

The RET-specific kinase inhibitor HM06/TAS0953 was observed to block growth of preclinical models of RET rearranged tumors that were never treated with a RET inhibitor as well as tumors that were resistant to the multi-kinase RET inhibitors cabozatinib and RXDX-105. The efficacy of HM06/TAS0953 was comparable to that of vandetanib. However, treatment with vandetanib resulted in significant weight loss in animals, an observation not seen with HM06. Importantly, HM06/TAS0953 suppressed growth of a preclinical model of RET fusion-positive lung cancer in the brain and extended overall survival.

### Example 6. Evaluation of HM06/TAS0953 Efficacy in an Orthotopic Xenograft Model of Lung Cancer Driven by RET Rearrangements in the Brain

The efficacy of HM06/TAS0953 to LOXO-292 and vandetanib in a brain orthotopic model of TRIM33-RET fusion-positive lung cancer was compared. Treatment of mice bearing tumors in the brain with HM06/TAS0953 (50 mg/kg BID) blocked tumor growth more effectively than LOXO-292 (10 mg/kg and 25 mg/kg BID dosages). HM06/TAS0953 caused a significant increase in survival of tumor-bearing mice compared to both doses of LOXO-292. Vandetanib (50 mg/kg QD) did not reduce tumor growth or survival of tumor-bearing animals. The results presented here suggest that HM06/TAS0953 is more effective than LOXO-292 in reducing growth or tumors in the brain.

Preparation of cells for injection into animal brain and quantitation of bioluminescence images: TRIM33-RET fusion cells were transduced with retroviruses harboring a GFP-luciferase construct and then GFP-positive cells were isolated by FACS. These cells were injected into the subcutaneous flank of NSG mice to generate xenograft tumors. Tumors were harvested and digested with a set of tumor dissociating enzymes (Miltenyi Biotech) in a GentleMACS tissue processor (Miltenyi Biotech) for 60 min and then digestion enzymes neutralized by adding growth media containing 10% FBS and then pelleted by centrifugation. Cells were then resuspended in fresh growth media, passed through a 75 μm filter, counted, washed once with PBS, and resuspended in PBS at a density of 100,000 cells/μL. Dissociated tumor cells were injected into the brain of anesthetized mice using a Hamilton syringe with a 26 G needle (1 μL) at the following coordinates: anterior (X): 0.5, posterior (Y): 1.5, dorsal (Z): 2.5. Wounds were sealed and mice were allowed to recover. Bioluminescence imaging was conducted weekly to monitor tumor growth and images were analysed with Image J software. The area (A) and mean intensity (I) of pixels in a region of interest (ROI) encompassing all luminescent regions of individual mice, identified using the Threshold function were measured. Mean background pixel intensity (B) was measured from non-luminescent areas. Total luminescence (L) for individual mice was quantified using the following equation: L=(I−B)×A, which adjusts for background intensity.

Mice were randomly assigned into groups of 6 mice after a robust signal was detected. Treatment was initiated 10 days after implantation of cells with vehicle, 50 mg/kg BID HM06, 100 mg/kg QD HM06/TAS0953 and 10 mg/kg BID LOXO-292. Treatment of mice with vandetanib (50 mg/kg QD) and 25 mg/kg BID LOXO-292 commenced 28 days after implantation as it took longer to obtain a robust bioluminescence signal from these mice. Bioluminescence was recorded weekly and animals were weighed twice weekly. Mice were sacrificed when signs of sickness such as a lack of coordination or excessive weight loss and fatigue were detected.

Vehicles and compounds: Vandetanib was mixed in 1% sodium carboxymethyl cellulose (CMC-Na) to generate a suspension. HM06/TAS0953 was mixed in 0.1 N HCl and 0.5% hypromellose (HPMC) to generate a suspension.

Statistical Analysis: Data sets were compared by Two-way Anova, with Tukey's or Sidaks' multiple comparison tests to determine significance. P<0.05 was considered a statistically significant difference between two values or data sets. Survival curves were compared using the Log-rank (Mantel-Cox) test. All graphs and statistical analyses were conducted using Graphpad Prism 8 software.

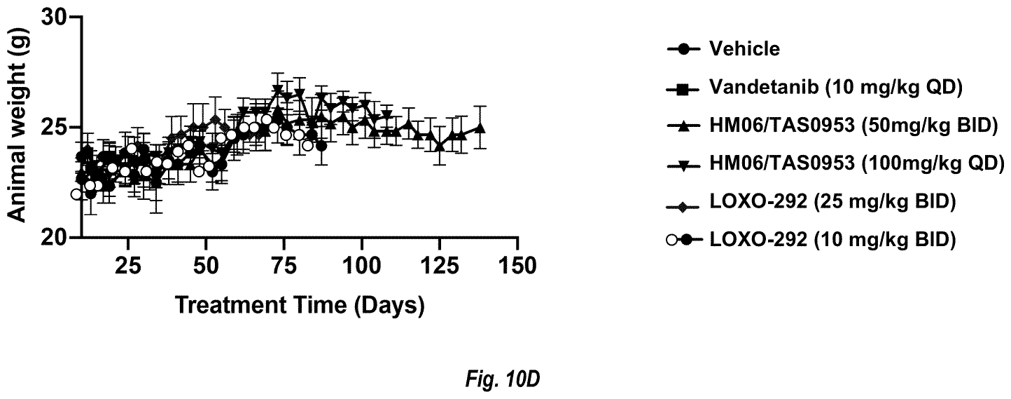

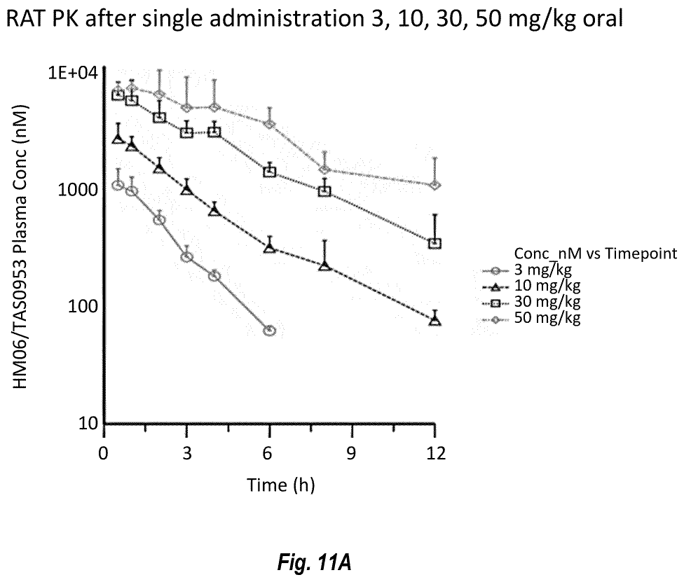

HM06/TAS0953 is more effective at inhibiting growth of a brain orthotopic xenograft tumor model with a RET fusion than LOXO-292: Vehicle-treated mice rapidly developed tumors as can be seen by the strong bioluminescence signal (FIGS. 10A and 10B) over the average starting signal. By 33 days of treatment tumor-bearing mice in the vehicle group were becoming sick and had to be sacrificed. In contrast, 5 days after treatment began there was a significant reduction in luciferase signal obtained from nice treated with 50 mg/kg BID (p=0.0012) or 100 mg/kg QD HM06/TAS0953 (p=0.0008). The 50 mg/kg HM06/TAS0953 BID dosage blocked tumor growth for the entire period of study (131 days of treatment). Treatment with LOXO-292 10 mg/kg BID did not cause any reduction in bioluminescence signal and tumors continued to grow while mice were being treated with LOXO-292 (FIG. 10B). The higher dose of LOXO-292 (25 mg/kg BID) reduced tumor growth for the first 3 weeks of treatment but then tumors continued to expand until mice were sacrificed due to high tumor burden, starting 64 days after treatment started. (FIG. 10B). Treatment with HM06/TAS0953 caused a significant increase in the survival of tumor bearing mice compared to 10 mg/kg BID LOXO-292 (p=0.0012) and 25 mg/kg BID LOXO-292 (p=0.001). At the end of the study there was low/undetectable luciferase signal in the 6 mice in the HM06/TAS0953 50 mg/kg BID group and all mice were still alive at the end of the study. There was no difference in survival between the two LOXO-292 groups. No treatment had any adverse effect on the weight of the experimental animals (FIG. 10D). These results confirm that HM06/TAS0953 crosses into the brain and is effective against RET-rearranged tumors that reside there.

The RET-specific kinase inhibitor HM06/TAS0953 blocked growth of lung cancer cells with RET fusion that were implanted into the brain of mice and resulted in increased survival of tumor-bearing animals. Given that LOXO-292 at a dosage of 10 mg/kg BID is sufficient to cause regression of RET fusion-positive tumors implanted in the subcutaneous flank of mice, these data indicate that there was poor delivery of LOXO-292 to the site of the tumor in the brain, even at the high dose of 25 mg/kg BID.

### Example 7. Safety Pharmacology Studies

Safety pharmacology studies were conducted for cardiovascular, respiratory, and central nervous system. HM06-01/TAS0953-01 did not exert any biologically relevant changes in the pro-convulsant activity and respiratory system.

Central Nervous System

Since HM06-01/TAS0953-01 readily crosses the blood brain barrier, effects on the CNS were assessed in several non-GLP and GLP safety pharmacology studies.

In a 2-week toxicology GLP study in rats, Irwin test was performed on day 1 at 1.5 hour after the second daily administration of HM06-01/TAS0953-01 at 25, 50 and 125 mg/kg BID. Each group comprised 10 male and 10 female rats. Animals dosed at 50 and 125 mg/kg BID showed decreased exploratory activity and arousal. In addition, females treated at 125 mg/kg BID had a higher mean number of urine pools (0.8 versus 0.0 in controls) and lower rectal temperature (37.1° C. versus 37.7° C. in controls). However, other Irwin test parameters were normal, and the health status of the animals was not grossly altered, therefore these changes were considered as not adverse.

To evaluate the potential proconvulsant and anticonvulsant effects, HM06-01/TAS0953-01 was administered at the dose level of 0, 30, 100 and 200 mg/kg SID via oral gavage to rats. Each group comprised 10 male rats. To assess the potential proconvulsant activity, oral dose levels of HM06-01/TAS0953-01 or 35 mg/kg of d-amphetamine were administered 1 hour prior to a subthreshold dose of 25 mg/kg PTZ to assess the proconvulsant effects. HM06-01/TAS0953-01 did not demonstrate any proconvulsant activity. No rats in the control or HM06-01/TAS0953-01 treatment groups that expressed a Stage 5, tonic/clonic convulsion. Whereas the positive control group in which rats were pretreated with d-amphetamine, displayed repetitive behaviors typical of dopamine agonist treatments in rats and the induction of full tonic/clonic convulsions in 3 out of 10 rats. To assess the relative anticonvulsant effects, HM06-01/TAS0953-01 or diazepam were administered orally to Wistar rats. HM06-01/TAS0953-01 at the doses of 100 and 200 mg/kg engendered a decrease in the total number of convulsions induced by the pre-load test of pentylenetetrazole. These data suggest that HM06-01/TAS0953-01 does not have proconvulsant activity and, noteworthy, that it may have anticonvulsant properties in the rat.

In a 2-week toxicity study and a 4-week toxicity study in dogs (further described in Example 8), CNS symptoms were seen in dogs after the first daily dose of ≥15 mg/kg BID during the routine clinical observations and at doses ≥5 mg/kg BID after the first administration in PK studies. In order to evaluate the potential neurotoxic effect of HM06-01, Fluoro-Jade C staining was performed on nine different brain areas (i.e., frontal pole, optic chiasm, infundibulum, mammillary bodies, base of third cranial nerve, anterior portion of pons and occipital pole section, cerebellum and medulla oblongata) of control dogs and dogs treated with 45 mg/kg BID HM06-01/TAS0953-01 (samples were taken from the 2-week toxicity study in dogs). In addition, ATF3 immunostaining was performed only on one control and one high dose dog treated with 45 mg/kg BID, in order to validate with an alternative method, the absence of any signs of neuronal degeneration or suffering. No difference between control dog and HM06-01/TAS0953-O1-treated dog brains were observed, meaning that there are no evidence of any neuronal degeneration and/or stressed/damaged neurons in any dog brain area studied.

Cardiovascular System

The effect of HM06-01/TAS0953-01 on the ECG was investigated in the 2-week toxicology GLP study in dog as described above on Day 12. No evidence of QT prolongation was seen after repeated administration of HM06-01/TAS0953-01 at the dose levels of 15, 30 and 45 mg/kg BID. Heart rate and ECG were also not affected at all dose levels tested. On the contrary, the females treated with the high dose showed lower arterial blood pressure at 5 hours after the first daily dosing and at 1 hour after the second daily dose. Mean values for mean arterial blood pressure were, respectively, 91 and 93 mmHg versus 127 and 115 mmHg in the vehicle group, and versus 119 mmHg in baseline. At +8 h, arterial blood pressure had returned to control/baseline levels. Although this decrease was not statistically significant versus control, it was consistent and noticeable enough to be considered test item related. Nevertheless, since the extent of the change was limited and present only in the high dose females, and as this decrease in blood pressure had no negative impact on the animals' health, it was considered as non-adverse.

The effect of HM06-01/TAS0953-01 on the ECG was also investigated in the 4-week toxicology GLP study in dog as described above (and further described in Example 8) on week 1 (Day 2), week 4 (Day 23) and at the end of the 2-week recovery period. On Day 2, two hours after the second daily dose, no effects of HM06-01/TAS0953-01 were noted at the dose levels of 15, 30 and 45 mg/kg BID. Whereas, on Day 23, no test item-related effects were noted on electrocardiographic parameters at 15 and 30 mg/kg BID in males. Conversely, a nonadverse and potentially test item-related slight increase in heart rate was noted at the dose level of 45 mg/kg BID in males and a test item and dose-related, slight (up to 8% versus control) prolongation of Fridericia and Van der Water corrected QTc interval was noted at the dose levels of 15, 30 and 45 mg/kg in females. These electrocardiographic effects were no longer observed at the end of the 2 week recovery period. In addition, there were no abnormalities in electrocardiogram rhythm and waveforms on Day 2 and Day 23, and at the end of the recovery period.

Respiratory System

The effect of HM06-01/TAS0953-01 on the respiratory system was also investigated after repeated administration with HM06-01/TAS0953-01 at 25, 50 and 90 mg/kg BID in the 4-week GLP study in rats further described in Example 8. Each group comprised 5 animals/group/sex. Neither toxicologically relevant effects in respiratory parameters were observed at week 4 nor delayed chronic effects were seen at week 6, two weeks after the end of the dosing period.

### Example 8. Four-Week Repeat-Dose Toxicity Studies in Rat and Dog

The adverse effects of HM06/TAS0953 were evaluated in the 4-week toxicity GLP studies in the rat and dog using the HM06/TAS0953 di-hydrochloride salt (HM06-01/TAS0953-01) mentioned above.

4-Week Tox Study in Rat

In the 4-week GLP toxicity study in rat, animals received 0, 25, 50, and 90 mg/kg BID HM06-01/TAS0953-01 by oral gavage. In the high dose of 90 mg/kg BID group, test item-related mortality was observed: 3 females were found dead or were prematurely sacrificed for ethical reasons (severe clinical signs like decreased activity, abnormal respiratory rate and erected fur) from Day 10 to Day 14. At necropsy, discolorations of the lungs were generally observed, histologically correlated with adverse moderate to marked inflammatory changes in the lungs (alveolar/perivascular inflammation, macrophages aggregates, alveolar oedema/hemorrhage). One additional female (satellite) was found dead on Day 7 before dosing. However, in absence of clinical sign and of abnormality at necropsy, the relationship with the test item administration was considered doubtful.

Test item-related clinical signs were observed in both sexes and with a dose-dependency such as abnormal respiratory rate, abnormal sounds during breathing, decreased/increased activity, locomotor stereotypy, hunched posture, low carriage, abnormal gait, pedaling, closed eyes, chewing action, abnormal pupil and cold to touch. These clinical observations were transient and with a higher incidence 30 minutes after dosing. All these signs did not persist in recovery animals after the end of dosing.

There was no effect on body weight at 25 mg/kg BID and 50 mg/kg BID whereas, at 90 mg/kg BID, a decrease of 10% body weight for both sexes associated to a lower body weight gain and a decrease in food consumption was observed. These effects were reversible at the end of the recovery period.

There was no change in ophthalmology and no toxicologically relevant acute and chronic effects in respiratory function evaluation. There was no effect on coagulation parameters.

At hematology, dose-dependent increase in reticulocytes in both sexes was seen. In addition, increased neutrophils in males only and increased platelets at mid and high dose male and high dose female were seen. A statistically higher white blood cells count was also observed in males at the high dose only. All these effects were reversible at the end of the recovery period.

Compared with the control group, the following changes in mean clinical chemistry parameters were seen with a dose-dependency and were considered as test item-related:
dose-dependent increase in AST and ALT in both sexes was seen.
higher AP in males at all doses
higher cholesterol and lower triglycerides were observed in males at mid and high dose.
   All these effects were reversible at the end of the recovery period.

A dose-dependent increase in urine protein concentration was noted in both sexes, with a higher incidence in the high dose males (7/10 males with 1 g/L) and with a higher incidence at mid and high dose females (4/10 females and 3/7 females with ≥1 g/L respectively). This effect was reversible at the end of the recovery period. The Tₘₐₓ was generally observed at 7 hours after the first daily dose (1 hour after the second daily dose) on Day 1 and at 1 hour after the first daily dose on Day 28 with a Cₘₐₓ increasing with the increase in dose levels. The T1/2 when estimable after the second daily dose, was observed ranging between 2.03 and 3.67 hours. Systemic exposure to HM06-01/TAS0953-01 increased with the increase dose levels in an approximately dose proportional manner and was similar between sexes. On Day 28 no systemic accumulation was noted.

At the end of dosing period, HM06-01/TAS0953-01-related inflammatory microscopic findings were noted in the lungs, pancreas (vacuolation/apoptotic acinar cells), femur (physeal hypertrophy), testes/epididymis (vacuolation of sertoli cells, tubular degeneration, increased intratubular cell debris, decreased sperm content). At the end of recovery period, test item-related findings seen at terminal sacrifice and premature decedents were fully reversible for the pancreas, and showed sporadic incidence with minimal severity in the lungs and femur mainly at 90 mg/kg BID indicating a progressive ongoing recovery. The persistence in recovery period of test item-related changes in the testes and epididymis with increased incidence and severity at 90 mg/kg BID, indicate an incomplete reversibility.

Based on these results, the severely toxic dose 10 (STD 10) was set at 50 mg/kg BID, corresponding to a mean AUC₍₀₋ₜ₎ on Day 1 of 50300 ng h/mL in males and 37200 ng h/mL in females and, on Day 28, of 73000 ng h/mL in males and 74300 ng h/mL in females.

4-Week Tox Study in Dog

In the 4-week GLP toxicity study in dog, animals received 0, 15, 30, and 45 mg/kg BID HM06-01/TAS0953-01 by oral gavage for 4 consecutive weeks followed by 2-week recovery period. In the high dose of 45 mg/kg BID group, test item-related mortality was observed: 2 males were prematurely sacrificed on Day 17 and Day 18 for ethical reasons (severe clinical signs associated with a significant body weight loss). The main cause of death was the mild to moderate multifocal subacute inflammation with alveolar or bronchioalveolar distribution, accompanied in one male with alveolar foreign body granulomas. In the other male, minimal pancreatic acinar vacuolation/apoptosis with prostatic acinar apoptosis were noted, for which the relationship with test item cannot be excluded.

Test item-related clinical signs were dose-dependent and noted starting from Day 1 at dose ≥15 mg/kg BID. These observations included decreased activity and tremors, lying on side, fur/skin staining, closed eyes, abnormal gait, cold to touch at all doses; vomiting and salivation, discolored urine/red feces, subdued/prostrate and apparent muscle atrophy (hind limb) at the mid and high dose levels. However, these clinical signs were transient with a higher incidence 30 minutes after dosing.

There was no effect on body weight at low dose whereas, body weight loss was noted between Day 1 and Day 29 at 30 and 45 mg/kg BID in females and in prematurely sacrificed males at 45 mg/kg BID (−25% for Male No. 644 and −12% for Male No. 642 at day 15 and 17 respectively). At the end of the recovery period, mid and high dose females gained weight, but their body weight gain was lower than the control animals.

There was no change in ophthalmology, urinalysis and on coagulation and clinical chemistry parameters.

At hematology, increase in total white blood cell count in both sexes due to an increase in monocyte count (at all doses with a dose dependency) and an increase in neutrophil count (at the high dose only in female, and at the intermediate and high doses in males); increase in reticulocyte count at the high dose in the males only, and increase in the platelet count in both sexes at the high and/or intermediate doses. A decrease in eosinophil count was considered doubtful considering that concerned only the females and was not dose-dependent. All these effects were reversible at the end of the recovery period, except for monocytes at the high dose in males and eosinophils in females.

The tₘₐₓ, was observed between 1 and 8 hours post first daily dose (1 hour post first daily dose and 2 hours post second daily dose). Based on AUC₍₀₋ₜ₎, systemic exposure to HM06/TAS0953 increased slightly more than dose-proportionally, except between 30 and 45 mg/kg BID in males and females on Day 28, where the increase was less than dose-proportional. Similar exposure to HM06/TAS0953 was observed between males and females on Days 1 and 28 at all the administered dose. The exposure to HM06/TAS0953 on Day 28 when compared to Day 1 was similar at 30 mg/kg BID and decreased or remained similar for some individuals at 15 and 45 mg/kg BID.

At the end of dosing period, a decreased mean weight of thymus in all females and a decreased mean weights of prostate gland in males at 30 mg/kg BID and in the only surviving male at 45 mg/kg BID were observed. At histopathology, HM06-01/TAS0953-01 induced inflammatory microscopic findings in the lungs (subacute inflammation with alveolar or bronchioalveolar distribution), pancreas (acinar cells vacuolation/apoptosis) and thymus (atrophy). At the end of recovery period, test item-related findings in the lungs showed a progressive ongoing recovery. Histological findings observed at the end of recovery period in the pancreas and thymus showed a similar severity and/or sporadic incidence in control and treated groups without sign to toxicity or altered function spontaneously encountered in the beagle dog studies.

Based on these results, the highest non severely toxic dose (HNSTD) was established to be 30 mg/kg BID in this study, corresponding to a mean AUC (0-t) on Day 1 of 21400 ng h/mL in males and 29200 ng h/mL in females and, on Day 28, of 22300 ng h/mL in males and 29100 ng h/mL in females.

### Example 9. Correlation of Free Plasma Concentration of HM06/TAS0953 to Free Concentration in the Brain and Cerebrospinal Fluid

The pharmacokinetics of HM06/TAS0953 in the prefrontal cortex, cerebrospinal fluid (CSF) and plasma of freely-moving adult male Han® Wistar rats following single dose oral administration of 3, 10, and 50 mg/kg of HM06/TAS0953 dihydrochloride salt (doses indicate the free base administered) was evaluated according to Table 3, below.

TABLE 3
Rat PK parameters after oral and IV administration in glucose 5%.

| | Dose | Half Life | Tₘₐₓ | Cₘₐₓ | AUC_{INF} | F | CL/F | V_{SS}/F |
|---|---|---|---|---|---|---|---|---|
| Group | (mg/kg) | (h) | (h) | (nM) | (nMh) | % | mL/min/kg | L/kg |
| | Oral Administration | | | | | | | |
| 1 | 3 | 1.3 | 0.5 | 1076 | 2509 | 47 | 36 | 4.6 |
| 2 | 10 | 2.5 | 0.5 | 2678 | 8255 | 46 | 34 | 6.1 |
| 3 | 30 | 2.8 | 0.5 | 6253 | 26569 | 49 | 32 | 7.5 |
| 4 | 50 | 3.7 | 1 | 7148 | 46162 | 51 | 32 | 10.2 |
| | Intravenous Administration | | | | | | | |
| 5 | 3 | 1.35 | 0.083 | 3985 | 5389 | 100 | 17 | 1.6 |

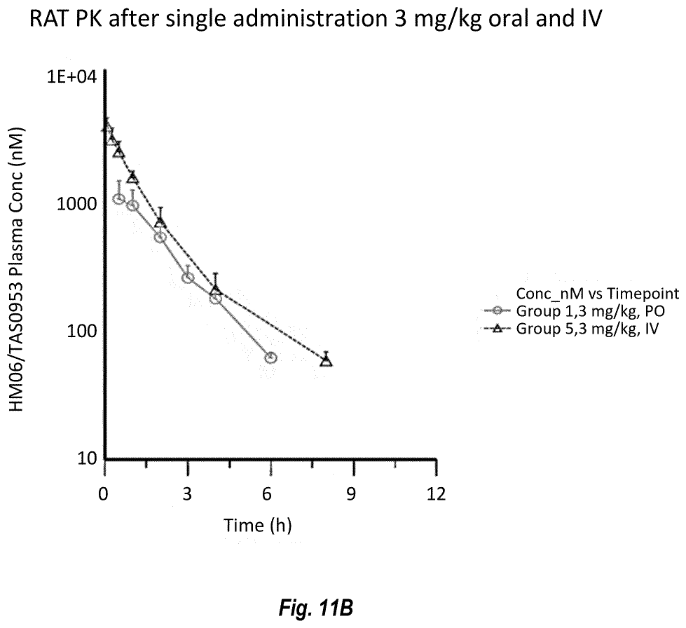

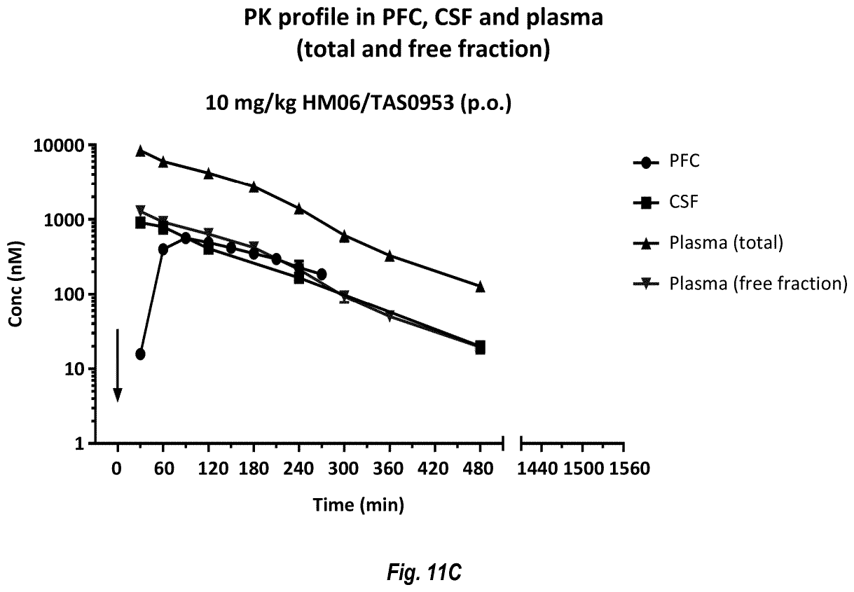

HM06/TAS0953 exposure in the prefrontal cortex (PFC), plasma, and cerebral-spinal fluid (CSF) increased with increasing dose in a largely dose-proportional manner over the used dose range from 3 to 50 mg/kg. Tmax 0.5-1 hour shows fast absorption of HM06/TAS0953 with short half-life, good F % (46-51%), moderate plasma clearance, large volume of distribution, and marginal renal clearance. FIG. 11A shows the plasma concentration over time following single oral administration of HM06/TAS0953 at 3, 10, 30, and 50 mg/kg. FIG. 11B shows the plasma concentration over time following single oral and intravenous administration of 3 mg/kg HM06/TAS0953.

Once equilibrium was reached between the compartments the ratio of the observed concentrations of HM06/TAS0953 in MetaQuant microdialysates from the PFC, CSF and plasma free fraction was close to 1:1:1. This concentrations ratio was maintained from 2 h until 6.5 h after HM06/TAS0953 administration (for CSF up to 8 h). FIG. 11C shows overlapping of the pharmacokinetic profile in plasma free fraction, PFC, and CSF from 2 h until 6.5 h after administration.

The 1:1 concentration ratio of free plasma to free brain concentration indicated that HM06/TAS0953 readily crosses the blood brain barrier. The free plasma concentration of HM06/TAS0953 can be used to estimate with a good approximation the free concentration in the brain and CSF. High brain penetrability properties may improve CNS outcomes (e.g., control, durability of response, and/or protection from CNS metastases).

### Example 10. Evaluation of HM06/TAS0953 Target Selectivity for RET

The selectivity for RET of HM06/TAS0953 was compared to LOXO-292 and BLU-667. As shown in Table 4, HM06/TAS0953 has higher kinase selectivity for RET as compared to LOXO-292 and BLU-667 based on IC₅₀ values.

TABLE 4
IC₅₀ values of kinase inhibition

| | | HM06/TAS0953 | LOXO-292 | BLU-667 |
|---|---|---|---|---|
| Kinase | | IC₅₀ (nM) | IC₅₀ (nM) | IC₅₀ (nM) |
| RET- | RET | 0.33 | 0.13 | 0.31 |
| neighboring | KDR | 172 | 14 | 35 |
| kinase | FLT1 | 2958 | 26 | 57 |
| | FLT3 | 911 | 22 | 11 |
| | FLT4 | 150 | 4.0 | 6.3 |
| | PDGFRα | 735 | 44 | 12 |
| | PDGFRβ | 2759 | 67 | 7.7 |
| | FGFR1 | 2724 | 64 | 15 |
| | FGFR2 | 1852 | 34 | 35 |
| | FGFR3 | 2152 | 51 | 58 |
| | DDR1 | 1192 | 103 | 7.1 |
| | DDR2 | >3000 | 417 | 7.7 |
| JAK family | JAK1 | >3000 | >3000 | 2.6 |
| kinase | JAK2 | >3000 | >3000 | 1.1 |
| | JAK3 | >3000 | 2838 | 6.1 |

From a safety perspective, the greater HM06/TAS0953 target selectivity for RET may minimize the adverse effects caused by potential off-target kinase inhibition, with a potential clinical benefit.

### Example 11. Starting Dose Determination for Human Trials

The starting dose calculation for human studies is based on data from the 4-week repeat-dose GLP-compliant toxicology studies, based on 1/10th of the Severely Toxic Dose in 10% (STD 10) of treated rats and ⅙th of the highest non-severely toxic dose (HNSTD) in dogs, as per ICH S9 guidelines.

The STD 10 in the 4-week rat toxicity study was set at 50 mg/kg BID. A dose of 50 mg/kg BID (equal to a 100 mg/kg daily dose) corresponds to a human equivalent dose (HED) of 8 mg/kg BID. Applying a safety factor of 10, the human starting dose could be as high as 0.8 mg/kg or 48 mg BID for a 60 kg body weight (BW) patient. An additional safety factor of 2.5 is added due to the dose-dependent alveolar inflammation and test item-related changes in the testes and epididymis observed in the 2- and 4-week toxicity study in rats, with progressive ongoing recovery and incomplete reversibility respectively at the end of the 2-week recovery period and the CNS effects observed in the FOB in rats after oral administration of ≥50 mg/kg SID. This leads to a proposed human starting dose of 20 mg BID.

The highest non-severely toxic dose in dogs (HNSTD) is the high dose tested in the 4-week toxicity study in dog (30 mg/kg BID). A dose of 30 mg/kg BID (equal to a 60 mg/kg daily dose) corresponds to a human equivalent dose (HED) of 16.2 mg/kg BID or 972 mg BID for a 60 kg BW patient. Applying a safety factor of 6, as specified in the ICH S9 guidance, the human starting dose could be as high as 2.7 mg/kg or 162 mg BID for a 60 kg patient. Consistently with the rat studies, an additional safety factor of 2.5 is applied due to the dose-dependent alveolar inflammation observed also in dogs with a progressive ongoing recovery at the end of the 2-week recovery period, the QTcF prolongation observed in the 4-week study in dogs and the CNS-related clinical signs noted during the general toxicity studies from Day 1 after the first administration at dose ≥15 BID mg/kg/day. This leads to a proposed human starting dose of 64.8 mg BID.

The starting dose for human trials was identified as 20 mg BID (i.e. 40 mg/day per patient) based on the data from the 4-week repeat-dose GLP-compliant toxicology studies. It is based on 1/10th of the Severely Toxic Dose in 10% (STD 10) of treated rats and ⅙th of the highest non-severely toxic dose (HNSTD) in dogs. An accelerated titration design (ATD) will permit not to expose too many patients to a potentially not effective dose, while administering study drug doses in the range of an acceptable tolerability.

### Example 12. Evaluation of Safety, Tolerability, Pharmacokinetics (PK) and Anti-Tumor Activity of HM06/TAS0953 in Patients with Advanced Solid Tumors with RET Gene Abnormalities

In order to evaluate the safety, tolerability, pharmacokinetics (PK) and anti-tumor activity of HM06/TAS0953, a Phase I/II open-label, single-arms, first-in-human study is performed. The study consists of two parts: a Phase I part with a Dose-Escalation and a Dose-Expansion cohort, and a Phase II part with 3 cohorts. Patients with advanced solid tumors harboring RET gene abnormalities will receive treatment. In both parts, a treatment cycle is 21 days of continuous dosing with no treatment interruption between cycles. Dosing will continue until disease progression, loss of clinical benefit, the development of unacceptable adverse events, start of new anticancer treatment, withdrawal of consent, physician's discretion, death, or lost-to-follow-up. The Phase I/II studies will also include safety assessment including evaluation of frequency, severity, and relatedness of TEAEs and serious adverse events (SAEs), changes in hematology and blood chemistry values, assessments of physical examinations, vital signs, and electrocardiograms (ECGs).

HM06/TAS0953 tablets will be administered orally BID (approximately every 12 hours) in fasting conditions (i.e., no food should be consumed in the interval between 2 hours before and 1 hour after drug administration).

The Phase I study will comprise oral treatment of HM06/TAS0953, starting dose 20 mg twice a day, until achieving a Maximum Tolerated Dose (MTD), continuous daily dosing, cycles lasting 21 days.

Based on preclinical efficacy data (ED₅₀ of oral 3 mg/kg/day in a mouse model), safety data (oral doses of 25 and 50 mg/kg BID in rats in the 4-week toxicology study), the human clearance of 6.4 mL/min/kg predicted by well-stirred modeling from recombinant CYPs data, and assuming a 50% oral bioavailability in humans, HM06/TAS0953 is expected to show signs of antitumor efficacy in cancer patients starting from approximately 40 mg BID with a HM06/TAS0953 daily exposure AUC_{0-24,ss}≥1650 ng*h/mL. The maximum administered dose may range from 500 mg BID to 1500 mg BID and provide a daily exposure AUC_{0-24,ss}≥22000 ng*h/mL and ≤63000 ng*h/mL. These exposures are equivalent, respectively, to those achieved at the safe dose of 25 mg/kg BID, lower than the STD10, and at the STD10 of 50 mg/kg BID in rats (measured AUC values in animals have been adjusted for humans by multiplying by the animal-to-human unbound fraction ratio). Based on the assumptions above, the maximum dose to be administered in this study may range from 500 mg BID to 1500 mg BID.

The Phase II study will comprise oral treatment, recommended dose twice a day, continuous daily dosing, cycles lasting 21 days.

Phase I Dose-Escalation

The Phase I Dose-Escalation study will follow an accelerated titration design (ATD), with an initial accelerated phase (1 patient per dose level) of 100% dose-step increments beginning with a starting dose of 20 mg BID. The accelerated phase converts to a 3±3 design after the 80 mg BID cohort. If in the first cycle of treatment (i.e. the first 21 days of treatment, Cycle 1) no Grade ≥2 drug related toxicity deemed as clinically meaningful by Safety Review Committee (SRC) or dose-limiting toxicities (DLTs) are observed in the accelerated phase, and 0/3 or <2/6 patients experience a DLT in the standard phase, dose escalation in new patients will continue until achieving a Maximum Tolerated Dose (MTD).

The first cohort will consist of 1 patient who will receive HM06/TAS0953 continuously for 21 days, in a 21-day cycle. If no relevant related toxicities are observed in this patient during cycle 1, the next cohort will be open; based on the trial design, one up to three cohorts will be included in the accelerated stage while the number of cohorts in the standard stage is not predictable as the dose escalation will continue until MTD is reached.

MTD is defined as: the highest dose level associated with less than or equal to 33% of patients experiencing a DLT at cycle 1.

DLT is defined as: treatment emergent toxicities in the first cycle of treatment, as detailed in the study protocol (except for AEs determined by Investigator to be clearly related to disease progression or intercurrent illness and unrelated to study drug).

Recommended Phase 2 Dose (RP2D) is defined as: dose to be tested in phase 2 based on overall safety, tolerability, PK data and estimates of efficacious exposures extrapolated from nonclinical data, derived from patients treated in the dose escalation and expansion dose levels. The RP2D may be equal or lower, but not higher, than the MTD. If the MTD is not reached, RP2D cannot be higher than the highest tested dose.

The primary objective of the Phase I Dose Escalation study is to determine the maximum tolerated dose (MTD) within the first 21 days of treatment (Cycle 1) and identify the recommended Phase 2 dose (RP2D).

The main secondary objectives of the Phase I Dose Escalation study are to evaluate the individual PK profiles of HM06/TAS0953 and its metabolites in plasma after single and multiple dosing (dense sampling); excretion in urine after single dosing; safety and tolerability; antitumor activity; and changes of RET gene status in circulating free nucleic acid obtained by liquid biopsies during treatment. The pharmacokinetic assessment of HM06/TAS0953 and metabolites may be assessed via measuring plasma concentration of HM06/TAS0953 and main metabolites and PK parameters, including but not limited to area under the curve from time 0 to 24 hours (AUC₀₋₂₄), maximum drug concentration (Cmax), time to maximum plasma concentration (Tmax), and degree of accumulation. Concentrations of HM06/TAS0953 in the cerebrospinal fluid (CSF) may be determined. A plasma sample may be collected at the same time to estimate the CSF/plasma ratio.

The study may include up to 36 patients evaluable for DLT assessment; the total number of patients will depend upon the number of dose escalations required and possible patient replacements. The target study population may include advanced solid tumor patients harboring RET gene abnormalities.

Phase I Dose Expansion:

In the Phase I Dose Expansion study, the RP2D dose level will be expanded with enrollment of additional patients. In a subset of patients, the food effect on HM06/TAS0953 bioavailability will be evaluated according to a randomized crossover design.

The primary objective of the Phase I Dose Expansion study is to confirm the recommended Phase 2 dose (RP2D) in the target population, to be used in the 3 phase II cohorts. The time frame dose escalation will be the first 21 days of treatment (Cycle 1) and every cycle (21 days) for approximately 10 months (or earlier if the patient discontinues from the study).

The main secondary objectives of the Phase I Dose Expansion study are to evaluate the individual PK profiles of HM06/TAS0953 and its metabolites in plasma at steady state by dense sampling in a subset of patients for individual PK characterization and by sparse sampling in all other patients for population PK analysis; food effect on HM06/TAS0953 bioavailability in a subset of patients; safety and tolerability; antitumor activity; and changes of RET gene status in circulating free nucleic acid obtained by liquid biopsies during treatment.

The study may include 20 to 30 patients, including at least 10 patients with measurable (according to RANO Working Group recommendation) CNS metastases at baseline. Preliminary food effect evaluation on HM06/TAS0953 bioavailability during Dose Expansion may occur in ten patients included in the PK evaluation.

The target study population includes locally advanced or metastatic NSCLC patients with primary RET gene fusion (with or without resistance mutations) and prior exposure to RET selective inhibitors. Patients must have documented progression of disease following existing therapies deemed by the Investigator to have demonstrated clinical benefit or unable to receive such therapies.

Phase II:

In the Phase II study, patients will be treated at the RP2D level in three cohorts: Cohort 1 and Cohort 2 (Pivotal) and Cohort 3 (Exploratory).

The primary objective of the Phase II study is to evaluate the antitumor activity (overall and intracranial if appropriate) of the selected RP2D in three different populations. Response rate will be assessed approximately every 6 weeks (±1 week) for the first 6 months, thereafter every 9 weeks (±1 week) in patients who have not progressed, until disease progression or study completion.

The main secondary objectives of the Phase II study are to evaluate safety and tolerability in three cohorts; the individual PK profiles of HM06/TAS0953 and its metabolites in plasma at steady state by sparse sampling in all patients for population PK analysis; and changes of RET gene status in circulating free nucleic acid obtained by liquid biopsies during treatment. Concentrations of HM06/TAS0953 in the cerebrospinal fluid (CSF) may be determined. A plasma sample may be collected at the same time to estimate the CSF/plasma ratio.

Additional secondary outcome measures may include ORR by investigator (objective response rate); disease control rate; time to tumor response; duration of response; time to progression; progression free survival; overall survival; as well as any of the above with specific regard to the central nervous system.

The Phase II study may include in Cohort 1: Fifty-five patients in a single stage design. The study may include in Cohort 2: Up to 61 patients according to a Simon two-stage design (24 patients in first stage to continue and 37 at second stage). The study may include in Cohort 3: three patients for each tumor type group. If clinical benefit is observed more patients may be enrolled.

The target study population in Cohort 1 (Pivotal) may include: Locally advanced or metastatic NSCLC patients with primary RET gene fusion (with or without resistance mutations) and prior exposure to RET selective inhibitors: with documented progression of disease following existing therapies deemed by the Investigator to have demonstrated clinical benefit or unable to receive such therapies; with and without measurable brain and/or leptomeningeal metastases.

The target study population in Cohort 2 (Pivotal) may include: Locally advanced or metastatic NSCLC patients with RET gene fusions naïve to RET selective inhibitors: with documented progression of disease following existing therapies deemed by the Investigator to have demonstrated clinical benefit or unable to receive such therapies; with and without measurable brain and/or leptomeningeal metastases.

The target study population in Cohort 3 (Exploratory) may include: Patients with advanced solid tumors that harbor RET gene abnormalities (other than NSCLC patients with primary RET gene fusions) and have failed all the available therapeutic options or in the opinion of the Investigator is unlikely to substantially benefit from other therapies and/or if the patient refuses.

Inclusion criteria for the Phase I/II studies may include:
Phase I—Common inclusion criteria for Dose-Escalation/Dose-Expansion:
   Male or female patient ≥18 years of age.
   Eastern Cooperative Oncology Group (ECOG) performance score of 0 or 1.
   Available RET-gene abnormalities determined on tissue biopsy or liquid biopsy at baseline.
   Adequate hematopoietic, defined as follows:
      Platelet count ≥100,000/μL
      Absolute neutrophil count ≥1,500/μL
      Hemoglobin level ≥9.0 g/dL (red blood cell transfusion and erythropoietin may be used to reach at least 9.0 g/dL but must have been administered at least 2 weeks prior to the first dose of study drug).
   Adequate hepatic function, defined as total serum bilirubin levels ≤1.5× upper limit of normal (ULN), serum albumin ≥2 g/dL, AST and ALT levels ≤2.5× ULN if no hepatic metastases are present; ≤5×ULN if hepatic metastases are present.
   Adequate renal function, defined as serum creatinine ≤2×ULN or estimated creatinine clearance ≥60 mL/min.
   Male and female patients of childbearing potential and at risk for pregnancy must agree to use a highly effective method of contraception from the date of the informed consent form's signature, throughout the study, and to be continued for 180 days after the last dose of assigned treatment. A patient is of childbearing potential if, in the opinion of the Investigator, he/she is biologically capable of having children and is sexually active.
Phase I Dose-Escalation—Specific inclusion criteria:
   Patient with advanced solid tumors with evidence of RET gene abnormalities.
   Measurable and/or non-measurable disease as determined by RECIST 1.1
   Patient must have documented progression of disease following at least one prior or more lines of therapy for advanced solid tumors or have exhausted all available therapies.
   If patient has brain and/or leptomeningeal metastases, he/she should be asymptomatic. Both measurable and nonmeasurable lesions according to RANO Working Group (WG) recommendations are permitted.
   If patient has been already treated with prior RET selective inhibitors, at least 5 half-lives must have elapsed from prior RET selective inhibitors treatment to first dose of HM06/TAS0953.
Phase I Dose-Expansion—Specific inclusion criteria:
   Patient with locally advanced or metastatic NSCLC patients with primary RET gene fusion (with or without resistance mutations) and prior exposure to RET selective inhibitors:
      with documented progression of disease following existing therapies deemed by the Investigator to have demonstrated clinical benefit or unable to receive such therapies;
      with or without brain and/or leptomeningeal metastases at baseline.
   If patient has brain and/or leptomeningeal metastases, he/she should have:
      asymptomatic untreated brain/leptomeningeal metastases off steroids and anticonvulsant for at least 7 days (these should be captured as target lesions if size eligible) or
      asymptomatic brain metastases already treated with local therapy (WBRT/SRT/surgery) and be clinically stable on steroids and anticonvulsant for at least 7 days before study drug administration.
   Measurable disease as determined by RECIST 1.1.
   At least 5 half-lives must have elapsed from prior RET selective inhibitors treatment to first dose of HM06/TAS0953.
   Either RET status documentation preferentially by NGS test managed locally or demonstrated substantial and durable (>6 months) clinical benefit from previous RET inhibitor treatment.
Phase II—Common inclusion criteria for Cohorts 1-3
   Male or female patient ≥18 years of age.
   Eastern Cooperative Oncology Group (ECOG) performance score of 0-2.
   Available RET-gene abnormalities determined on tissue biopsy or liquid biopsy at baseline.
   Measurable disease as determined by RECIST 1.1.
   If patient has brain and/or leptomeningeal metastases, he/she should have:
      asymptomatic untreated brain/leptomeningeal metastases off steroids and anticonvulsant for at least 7 days (these should be captured as target lesions if size eligible) or
      asymptomatic brain metastases already treated with local therapy (WBRT/SRT/surgery) and be clinically stable on steroids and anticonvulsant for at least 7 days before study drug administration.
   Adequate hematopoietic, defined as follows:
      Platelet count ≥100,000/μL
      Absolute neutrophil count ≥1,500/μL
      Hemoglobin level ≥9.0 g/dL (red blood cell transfusion and erythropoietin may be used to reach at least 9.0 g/dL but must have been administered at least 2 weeks prior to the first dose of study drug).
   Adequate hepatic function, defined as total serum bilirubin levels ≤1.5×ULN, serum albumin ≥2 g/dL, AST and ALT levels ≤2.5×ULN if no hepatic metastases are present; ≤5×ULN if hepatic metastases are present.
   Adequate renal function, defined as serum creatinine ≤2×ULN or estimated creatinine clearance ≥60 mL/min.
   Male and female patients of childbearing potential and at risk for pregnancy must agree to use a highly effective method of contraception from the time of the first negative pregnancy test at screening, throughout the study and for 180 days after the last dose of assigned treatment. A patient is of childbearing potential if, in the opinion of the Investigator, he/she is biologically capable of having children and is sexually active.
Phase II Cohort 1—Specific inclusion criteria:
   Locally advanced or metastatic NSCLC patient with primary RET gene fusion (with or without resistance mutations) and prior exposure to RET selective inhibitors.
      Documented progression of disease following existing therapies deemed by the Investigator to have demonstrated clinical benefit or unable to receive such therapies.
   At least 5 half-lives must have elapsed from prior RET selective inhibitors treatment to first dose of HM06/TAS0953.
   Either RET status documentation preferentially by NGS test managed locally or demonstrated substantial and durable (>6 months) clinical benefit from previous RET inhibitor treatment.
Phase II Cohort 2—Specific inclusion criteria:
   Locally advanced or metastatic NSCLC patient with RET gene fusion without prior exposure to RET selective inhibitors.
   Documented progression on disease following existing therapies deemed by the Investigator to have demonstrated clinical benefit or unable to receive such therapies.
Phase II Cohort 3—Specific inclusion criteria:
   Patient with advanced solid tumors that harbour RET gene abnormalities (other than NSCLC patients with primary RET gene fusions) and has failed all the available therapeutic options or in the opinion of the Investigator is unlikely to substantially benefit from other therapies and/or if the patient refuses; this could include (but not limited to):
      Medullary or anaplastic thyroid cancer who have progressed or developed intolerance to selective RET inhibitors;
      Metastatic breast cancer;
      Metastatic pancreatic adenocarcinoma;
      NSCLC with emerging RET pathway after, but not limited to, EGFR/ALK/ROS1/BRAF targeted therapies.
   If patient has been already treated with prior RET selective inhibitors, at least 5 half-lives must have elapsed from prior RET selective inhibitors treatment to first dose of HM06/TAS0953.

Exclusion Criteria for the Phase I/II studies may include:
Phase I—Common exclusion criteria for Dose-Escalation/Dose-Expansion:
   Lactating woman.
   Investigational agents or anticancer therapy within 5 half-lives (or 1 half-life for long-lasting drugs such as anticancer antibodies and other biologic drugs, provided there are no residual toxicities) prior to the first dose of study drug.
   Major surgery (excluding placement of vascular access) within 4 weeks prior to the first dose of study drug or planning to undergo major surgery during the course of study treatment.
   Patient who has received WBRT within 14 days or other palliative radiotherapy within 7 days prior to the first dose of study drug, or who has not recovered from side effects of such therapy, if in the opinion of the Investigator this is clinically meaningful.
   Patient with a primary CNS tumor.
   Clinically significant, uncontrolled, cardiovascular disease including myocardial infarction within 3 months prior to Day 1 of Cycle 1, unstable angina pectoris, significant valvular or pericardial disease, history of ventricular tachycardia, symptomatic Congestive Heart Failure (CHF) New York Heart Association (NYHA) class III-IV, and severe uncontrolled arterial hypertension, according to the Investigator's opinion.
   Sustained over time QT interval corrected using Fridericia's formula (QTcF) >470 msec; personal or family history of prolonged QT syndrome or history of Torsades de pointes (TdP). History of uncontrolled and persistent risk factors for TdP (e.g., heart failure, hypokalemia, use of concomitant medications that prolong the QT/QTc interval despite optimal treatment).
   Current active infection with HIV, hepatitis B virus, or hepatitis C virus.
   Documented history of interstitial lung disease or current evidence of interstitial lung disease that requires steroid medication.
   Clinically significant gastrointestinal abnormality that would affect drug absorption according to the Investigator's opinion.
   Clinically significant diseases affecting digestive function (including chronic diarrhea) that in the opinion of the Investigator would affect study drug therapy.
   Treatment with strong CYP3A4 inhibitors within 1 week (7 days) prior to the first dose of study drug.
   Repeated treatment with strong CYP3A4 inducers within 3 weeks (21 days) prior to the first dose of study drug.
   Known hypersensitivity to additives in HM06/TAS0953.
   Any illness or medical condition that, in the opinion of the Investigator, may confound the results of the study or pose unwarranted risks in administering the investigational product to the patient.
Phase I Dose-Escalation—Specific exclusion criteria:
   Patient with symptomatic CNS metastases at baseline.
Phase I Dose-Expansion—Specific exclusion criteria:
   Patient with symptomatic brain and/or leptomeningeal metastasis at baseline, not controlled by local and/or systemic therapy.
   Presence of known EGFR, KRAS, ALK, HER2, ROS1, BRAF and METex14 activating mutations.
Phase II—Common exclusion criteria for Cohorts 1-3:
   Lactating woman.
   Investigational agents or anticancer therapy within 5 half-lives (or 1 half-life for long-lasting drugs such as anticancer antibodies or other biologic drugs, provided there are no residual toxicities) prior to the first dose of study drug.
   Major surgery (excluding placement of vascular access) within 4 weeks prior to the first dose of study drug or planning to undergo major surgery during the course of study treatment.
   Patient who has received WBRT within 14 days or other palliative radiotherapy within 7 days prior to the first dose of study drug, or who has not recovered from side effects of such therapy, if in the opinion of the Investigator this is clinically meaningful.
   Patient with a primary CNS tumor.
   Patient with symptomatic brain and/or leptomeningeal metastasis at baseline, not controlled by local and/or systemic therapy.
   Clinically significant, uncontrolled, cardiovascular disease including myocardial infarction within 3 months prior to Day 1 of Cycle 1, unstable angina pectoris, significant valvular or pericardial disease, history of ventricular tachycardia, symptomatic Congestive Heart Failure (CHF) New York Heart Association (NYHA) class III-IV, and severe uncontrolled arterial hypertension, according to the Investigator's opinion.
   Sustained over time QT interval corrected using Fridericia's formula (QTcF) >470 msec; personal or family history of prolonged QT syndrome or history of Torsades de pointes (TdP). History of uncontrolled and persistent risk factors for TdP (e.g., heart failure, hypokalemia, use of concomitant medications that prolong the QT/QTc interval despite optimal treatment).
   Current active infection with HIV, hepatitis B virus, or hepatitis C virus.
   Documented history of interstitial lung disease or current evidence of interstitial lung disease that requires steroid medication.
   Clinically significant gastrointestinal abnormality that would affect drug absorption according to the Investigator's opinion.
   Clinically significant diseases affecting digestive function (including chronic diarrhea) that in the opinion of the Investigator would affect study drug therapy.
   Treatment with strong CYP3A4 inhibitors within 1 week (7 days) prior to the first dose of study drug.
   Repeated treatment with strong CYP3A4 inducers within 3 weeks (21 days) prior to the first dose of study drug.
   Known hypersensitivity to additives in HM06/TAS0953.
   Any illness or medical condition that, in the opinion of the Investigator, may confound the results of the study or pose unwarranted risks in administering the investigational product to the patient.
Phase II Cohort 1—Specific exclusion criteria:
   Presence of known EGFR, KRAS, ALK, HER2, ROS1, BRAF and METex14 activating mutations.
Phase II Cohort 2—Specific exclusion criteria:
   Presence of known EGFR, KRAS, ALK, HER2, ROS1, BRAF and METex14 activating mutations.
Phase II Cohort 3—Specific exclusion criteria:
   None.

Drug Administration:

HM06/TAS0953 is prepared as di-hydrochloride salt (HM06-01/TAS0953-01) for clinical use. The product is formulated as tablets for oral use. The studies are conducted with tablets at a dose of 10 and 50 mg/unit (expressed as free base). The intended storage condition is refrigerated condition (controlled temperature between 2° and 8° C. [36° and 46° F.]).

HM06/TAS0953 tablets will be administered orally BID (approximately every 12 hours) in fasting conditions (i.e., no food should be consumed in the interval between 2 hours before and 1 hours after drug administration) with a 20 mg (starting dose) BID.

### Example 13. Dose-Escalation Part of Phase I Study Progresses to 320 mg BID Dose Level

While the escalation part of the Phase I study is still in progress, safety information from the first cycle of treatment in patients administered 160 mg BID has allowed for the dose escalation to the 320 mg BID dose level. This process complies with the study protocol outlined in Example 12 above, i.e., when safety data from the first cycle of treatment to the patients recruited as per protocol in a specific dose level are available, a Safety Review Committee (SRC) meeting is held to escalate the dose. As discussed in Example 12 above, the maximum dose to be administered in this study may range from 500 mg BID to 1500 mg BID.

### Example 14. Cellular Potency of HM06/TAS0953 Against RET Solvent Front Mutations

Although selective RET inhibitors LOXO-292 (selpercatinib) and BLU-667 (pralsetinib) show clinical antitumor activity in non-small cell lung cancers, acquired resistances driven by RET solvent front mutations (e.g., RET^{G810R/S/C}) have emerged. (See Subbiah, V et al., Ann Oncol. 2021, 32(2):261-268; Lin J J et al., Ann Oncol. 2020, 31(12):1725-1733; Solomon B J et al., J Thorac Oncol. 2020, 15(4):541-549; Fancelli S et al., Cancers (Basel). 2021, 13(5):1091. doi: 10.3390/cancers13051091.) In such cases, no other treatment options are currently available for these patients after relapse with selpercatinib or pralsetinib.

Cellular potencies against RET wildtype fusions and RET mutations, including the gatekeeper mutations V804L/M and selpercatinib- or pralsetinib-resistant mutations G810R/S, were investigated in engineered Ba/F3 cells. About 1,000 cells per well were cultured in 96-well plates, and were treated with HM06/TAS0953, vandetanib, BLU-667, or LOXO-292 for 72 hours (at 37° C., 3 days). Cell viability was assessed by luminescence using the CellTiter-Glo 2.0 Assay (Promega Corporation). GI50 value (the concentration that exerted 50% growth inhibition compared with that of the compound-untreated controls) were calculated using a sigmoidal dose response model in XLfit software (ID Business Solutions). Data are presented as mean±SD of the data obtained from three independent experiments.

The IC50 data is shown in Table 5. Ba/F3 KIF5B-RET^{G810R/S} cells were found to be resistant not only to BLU-667 but also to LOXO-292. However, Ba/F3 KIF5B-RETG^{G810R/S} cells were sensitive to HM06/TAS0953.

In addition, Ba/F3 KIF5B-RET^{V804L/M} cells were sensitive to HM06/TAS0953, BLU-667, and LOXO-292, but were not to vandetanib.

TABLE 5

| | GI₅₀ values (nmol/L) | | | |
|---|---|---|---|---|
| Cell lines | HM06/TAS0953 | Vandetanib | BLU-667 | LOXO-292 |
| Ba/F3 | >10000 | 7490 ± 1560 | 1350 ± 250 | 3980 ± 470 |
| Ba/F3 KIF5B-RET | 8.27 ± 1.29 | 209 ± 76 | 6.51 ± 2.13 | 6.68 ± 0.76 |
| Ba/F3 KIF5B-RET V804L | 21.5 ± 4.4 | 4050 ± 485 | 7.18 ± 3.15 | 7.13 ± 1.50 |
| Ba/F3 KIF5B-RET V804M | 39.7 ± 4.6 | 3380 ± 246 | 8.60 ± 0.58 | 33.3 ± 2.9 |
| Ba/F3 KIF5B-RET G810R | 43.8 ± 18.3 | 3760 ± 460 | 1140 ± 300 | 1020 ± 200 |
| Ba/F3 KIF5B-RET G810S | 11.9 ± 3.6 | 2880 ± 420 | 50.6 ± 31.4 | 166 ± 20 |

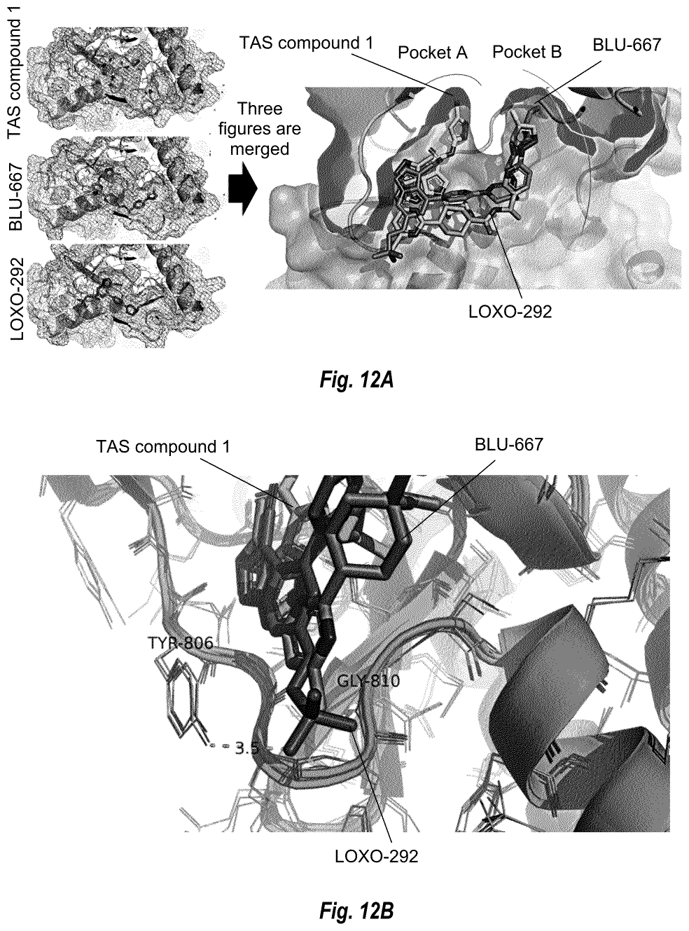

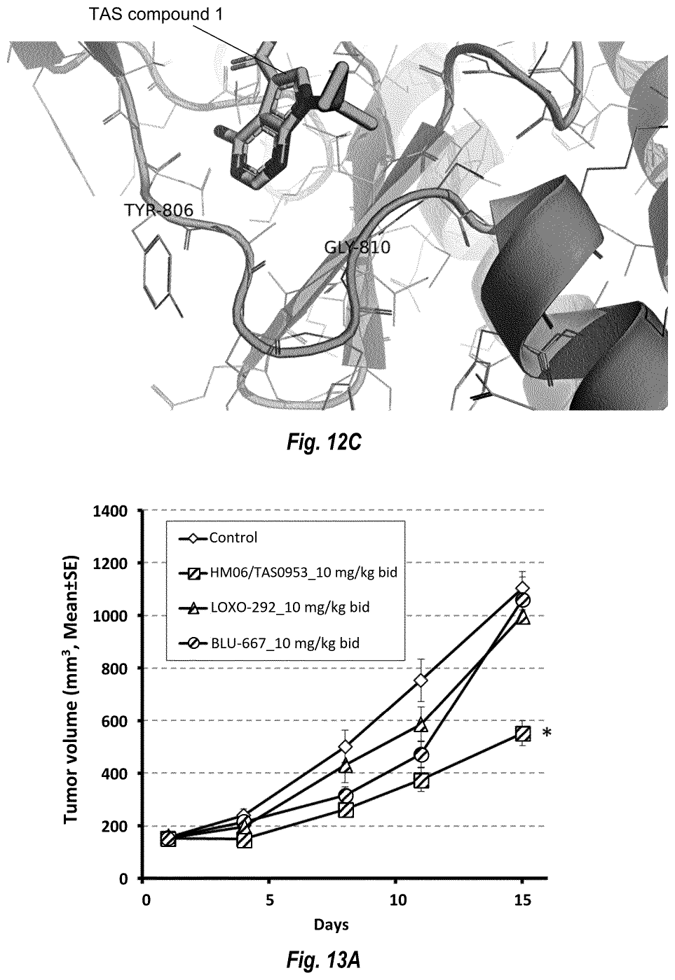

To help elucidate the mechanism by which RET^{G810R/S} mutants remain sensitive to HM06/TAS0953, crystal structures of the RET kinase domain complexed with TAS compound 1 (having a structure similar to HM06/TAS0953), BLU-667, and LOXO-292 were examined. X-ray crystal structures of the complexes revealed that TAS compound 1 has unique binding mode to RET compared to BLU-667 and LOXO-292. Based on co-crystal structural data, as shown in FIG. 12A, BLU-667 and LOXO-292 bind to the same pocket in RET (Pocket B) whereas TAS compound 1 binds to a different pocket in RET (Pocket A) with a different binding mode. TAS compound 1 does not fill the space in the direction of the side chain of G810, suggesting that HM06/TAS0953 may effectively circumvent steric hindrance from solvent front substitutions. This feature likely contributes to the ability of HM06/TAS0953 to maintain its biological potency in G810 mutations.

The structural formula of TAS compound 1 is:

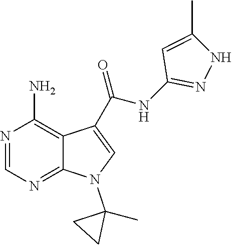 [SMILES: Cc1cc(NC(=O)c2cn(C3(C)CC3)c3ncnc(N)c23)n[nH]1]

FIGS. 12B and 12C show enlarged views of the structural analysis of RET co-crystal complexes in the region of RET amino acid residues 806-810. FIG. 12B shows co-crystal complexes of RET and TAS compound 1, BLU-667, and LOXO-292 and FIG. 12C shows the co-crystal complex of RET and TAS compound 1. Glycine 810 is close to both BLU-667 and LOXO-292 and a substitution at that position may affect the steric hindrance against those inhibitors. In contrast, TAS compound 1 does not insert into the pocket consisting of amino acid residues 806-810.

The data is consistent with the biological data that the inhibition pattern against RET mutation (e.g., G810R/S) is different between HM06/TAS0953 and BLU-667/LOXO-292.

### Example 15. Pharmacologic Efficacy of HM06/TAS0953 Against RET Mutants

The sensitivity of various RET mutations, including solvent front KIF5B-RET^{G810R/A/C/D/S} mutants, to 1 HM06/TAS0953, BLU-667, and LOXO-292 was examined in transiently transfected HEK293 cells by In-Cell Western™ (ICW) analysis of KIF5B-RET phosphorylation. IC50 values were calculated based on three separate experiments and the data summarized in Table 6, below.

The potency of HM06/TAS0953 against wild-type KIF5B-RET was equivalent to that of BLU-667 and LOXO-292. The pattern of the RET mutations inhibited by HM06/TAS0953 was different from those inhibited by LOXO-292 and BLU-667. HM06/TAS0953 inhibited G810X mutations, with the IC50 values of HM06/TAS0953 for suppressing the phosphorylation of RET^{G10X} ranging from 35.4 to 282 nmol/L. The potencies of HM06/TAS0953 against G810X mutations were higher than those of BLU-667 and LOXO-292. Additionally, the potencies of HM06/TAS0953 against L730X, G736A, L760Q, L772M, Y806C, and A883V were also higher than those of BLU-667 and LOXO-292, whereas the potencies of HM06/TAS0953 against I788N and L865V were lower than those of BLU-667 and LOXO-292. The data suggest that HM06/TAS0953 may be effective against point mutations of KIF5B-RET that show resistance to other RET inhibitors, such as LOXO-292 and/or BLU-667.

Generation of Expression Vector

The expression vector was generated with Gateway Technology. First, the entry vector (pENTR/KIF5B-RET) was constructed using KIF5B-RET PCR products, pDONR 221 vector (Invitrogen Corporation), and Gateway BP Clonase Enzyme Mix (Invitrogen Corporation). Then, the KIF5B-RET expression vector was constructed using the entry vector, pJTI FAST K02-PuroR expression vector which was modified in Taiho Pharmaceutical Co., Ltd. using pJTI-FAST DEST expression vector (Thermo Fisher Scientific) and Gateway LR Clonase Enzyme Mix (Invitrogen Corporation).

In-Cell Western

Jump-In GripTite HEK293 cells were transiently transfected with the KIF5B-RET expression vector using TransIT-X2 (Mirus Bio LLC.). After treatment with each test compound at various concentrations for 1 hour, cells were fixed with 20% Formalin Neutral Buffer solution. Then the microplates were blocked with Intercept© (PBS) Blocking Buffer (LI-COR Inc.) for 1 hour at room temperature, and incubated overnight at 4° C. with the primary antibodies (anti-phospho-RET (Tyr905) antibody (Catalog No. 3221, Cell Signaling Technology, Inc.) and anti-RET antibody (Catalog No. sc-101422, Santa Cruz Biotechnology, Inc.)) diluted with Intercept© (PBS) Blocking Buffer. Microplates were subsequently washed and incubated with the secondary antibodies (goat anti-rabbit IRDye 800CW and goat anti-mouse IRDye 680RD) (LI-COR Inc.).

After washing the microplates, an Odyssey CLx Imaging System (LI-COR Inc.) was used to quantify the fluorescence of each well. T/C (%) was determined as follows:

(Mean signal of test compound)/(Mean signal of Control)×100Mean signal:(Fluorescence of phospho-RET−background)/(Fluorescence of RET−background)

IC50 value was calculated by fitting from concentration vs. T/C (%) curve using XLFit. IC50 value was determined by three separate experiments.

TABLE 6
Summary of IC₅₀ values

| | IC₅₀ values (nmol/L) | | |
|---|---|---|---|
| Gene | HM06/TAS0953 | BLU-667 | LOXO-292 |
| KIF5B-RET | 80.2 ± 14.7 | 74.5 ± 14.0 | 76.9 ± 18.0 |
| KIF5B-RET_L730Q | 60.2 ± 14.3 | 180 ± 28 | 546 ± 113 |
| KIF5B-RET_L730R | 53.8 ± 14.6 | 1570 ± 330 | >3000 |
| KIF5B-RET_E732K | 70.1 ± 24.5 | 113 ± 53 | 65.6 ± 18.5 |
| KIF5B-RET_G736A | 20.7 ± 5.8 | 1060 ± 200 | 147 ± 40 |
| KIF5B-RET_M759I | 12.3 ± 3.5 | 11.6 ± 6.6 | 23.9 ± 4.4 |
| KIF5B-RET_L760Q | 27.1 ± 7.0 | 259 ± 53 | 228 ± 81 |
| KIF5B-RET_E768Q | 17.1 ± 5.3 | 24.2 ± 18.4 | 23.1 ± 10.1 |
| KIF5B-RET_L772M | 18.7 ± 3.2 | 43.6 ± 8.7 | 24.2 ± 3.9 |
| KIF5B-RET_I788N | 1640 ± 250 | 37.0 ± 6.0 | 28.0 ± 7.2 |
| KIF5B-RET_Y806C | 153 ± 25 | 457 ± 68 | 536 ± 75 |
| KIF5B-RET_L865V | 433 ± 171 | 60.6 ± 21.4 | 73.7 ± 29.8 |
| KIF5B-RET_A883V | 55.4 ± 9.5 | 371 ± 115 | 46.2 ± 16.7 |
| KIF5B-RET_G810A | 35.4 ± 5.6 | 125 ± 13 | 1330 ± 10 |
| KIF5B-RET_G810C | 282 ± 51 | 1520 ± 300 | >3000 |
| KIF5B-RET_G810D | 179 ± 108 | 640 ± 312 | >2670 |
| KIF5B-RET_G810S | 42.6 ± 4.7 | 232 ± 66 | >2220 |
| KIF5B-RET_G810R | 149 ± 39 | >3000 | >3000 |

### Example 16. Evaluation of Antitumor Effects of HM06/TAS0953 in Nude Mice Subcutaneously Implanted with Ba/F3 KIF5B-RET^{G810R} Cells

HM06/TAS0953 showed a significant antitumor effect and marked target inhibition in a KIF5B-RET^{G810R} animal tumor model suggesting that HM06/TAS0953 may be effective in treating tumors harboring the KIFSB-RET gene with the G810R mutation. At doses as low as 10 mg/kg twice a day, HM06/TAS0953 significantly inhibited tumor growth and phospho-RET. In addition to its selective high potency against wildtype RET, HM06/TAS0953 shows significant potency against G810R solvent front mutation, which is highly resistant to selpercatinib and pralsetinib, in vitro and in vivo.

In a first study, Ba/F3 KIF5B-RET^{G810R} cells (5×10⁶ cells/mouse) were suspended in 50% matrigel (Corning Incorporated)/PBS and were implanted subcutaneously into male athymic nude mice (BALB/cAJcl-nu/nu, CLEA Japan, Inc.). HM06/TAS0953, LOXO-292, and BLU-667 were formulated in 0.5 w/v % IPMC (Shin-Etsu Chemical Co., Ltd.) containing 0.1 mol/L HCl. One week after implantation, the mice were randomized to different treatment groups to equalize the mean tumor volume in each group, and orally administered vehicle (bid), HM06/TAS0953 (10, 30 mg/kg, bid), LOXO-292 (10, 30 mg/kg, bid) or BLU-667 (10, 30 mg/kg, bid) for 14 days. The group administered the vehicle of 0.5 w/v % IPMC containing 0.1 mol/L HCl was the control group. The length and width of tumors were measured using digital caliper (Mitutoyo) and tumor volume was calculated as follows: [length× (width)²]/2. The tumor volume and the body weights of mice were measured twice a week until the end of the study.

Statistical significance was determined by using Dunnett's test to compare the tumor volume in the treated groups with that of the control group. The statistical analysis was performed using SAS version 9.4 (SAS Institute Japan) via EXSUS Version 10.0 (CAC Exicare Corporation). P-values less than 0.05 were considered to indicate statistical significance.

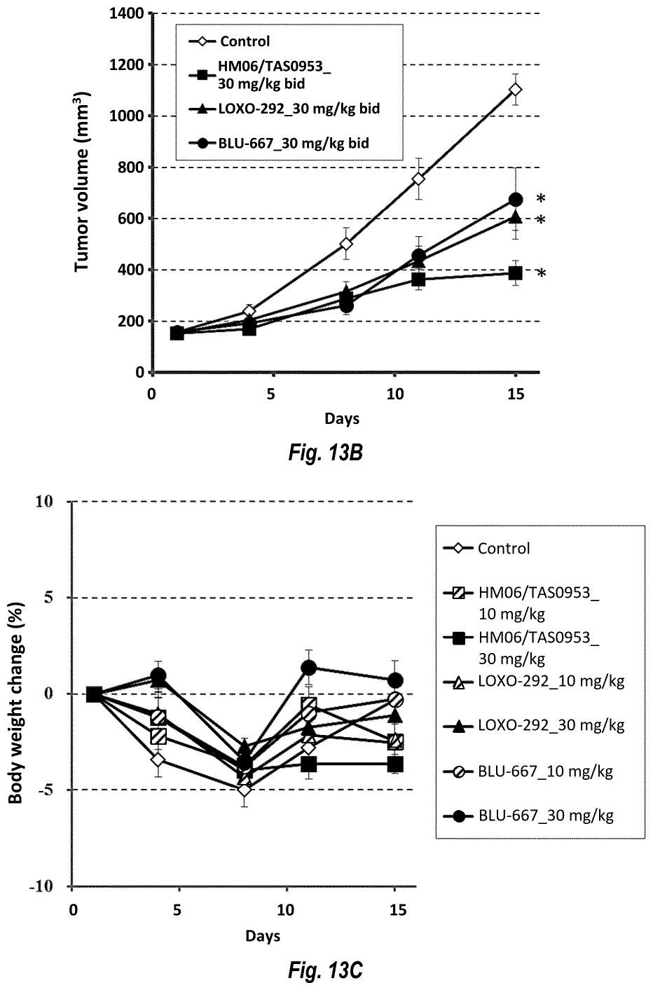

FIGS. 13A and 13B show the effects of HM06/TAS0953, LOXO-292, and BLU-667, each administered twice-daily at a low dose of 10 mg/kg (FIG. 13A) and a high dose of 30 mg/kg (FIG. 13B), on tumor volume. Data were presented as mean±SE (each group, n=5). As reflected in FIG. 13A, among the low-dose treatment groups, only the group treated with 10 mg/kg HM06/TAS0953 showed significantly decreased tumor volume at the end of the study compared to the control group (* reflects p<0.05; Dunnett test). As shown in FIG. 13B, at the end of the study, tumor volume in each of the high-dose treatment groups was reduced compared to the control group (* reflects p<0.05; Dunnett test). Moreover, as shown in FIG. 13B, at the end of the study, only the HM06/TAS0953 group treated at 30 mg/kg did not show tumor regrowth, while the LOXO-292 and the BLU-667 groups exhibited a trend to tumor regrowth in parallel with the control group. FIG. 13C shows the effects of the dosages above on body weight during the treatment period. There were no significant changes in terms of body weight change among groups. Data were presented as mean±SE (each group, n=5). One mouse in the BLU-667 30 mg/kg group died due to accident.

In a second study, Ba/F3 KIF5B-RET^{G810R} cells (5×10⁶ cells/mouse) were suspended in 50% matrigel (Corning Incorporated)/PBS and were implanted subcutaneously into 6-week-old male athymic nude mice (BALB/cAJcl-nu/nu, CLEA Japan, Inc.). HM06/TAS0953, LOXO-292 and BLU-667 were formulated in 0.5 w/v % HPMC (Shin-Etsu Chemical Co., Ltd.) containing 0.1 mol/L HCl. Five days after implantation, the mice were randomized to different treatment groups to equalize the mean tumor volume in each group, and orally administered vehicle (bid), HM06/TAS0953 (50 mg/kg, bid), LOXO-292 (30 mg/kg, bid) or BLU-667 (30 mg/kg, bid) for 14 days. The group administered the vehicle of 0.5 w/v % HPMC containing 0.1 mol/L HCl was the control group. The length and width of tumors were measured using digital caliper (Mitutoyo) and tumor volume was calculated as follows: [length× (width)2]/2. The tumor volume and the body weights of mice were measured twice a week until the end of the study.

Statistical significance was determined by using Dunnett's test to compare the TV in the treated groups with that of the control group. The statistical analysis was performed using SAS version 9.4 (SAS Institute Japan) via EXSUS Version 10.0 (CAC Exicare Corporation). P-values less than 0.05 were considered to indicate statistical significance.

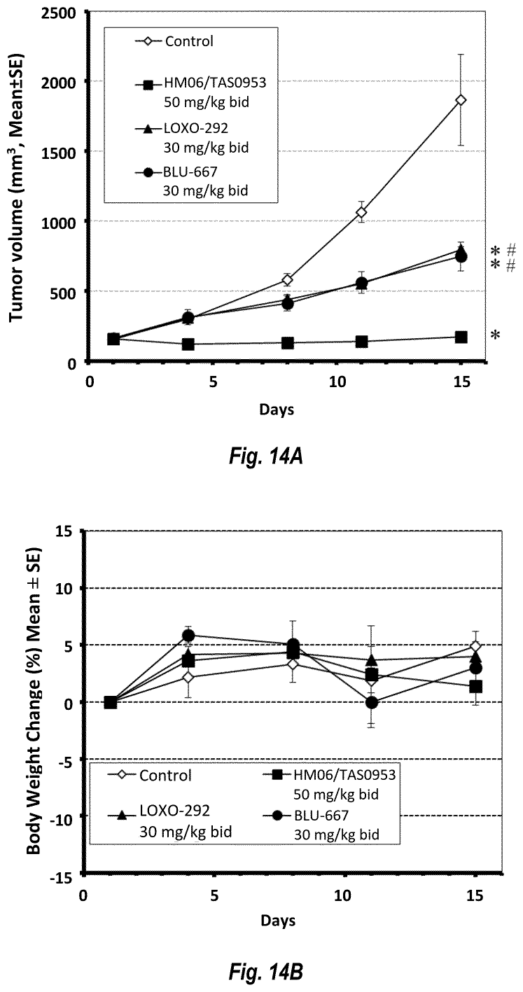

FIG. 14A shows the effects of HM06/TAS0953, LOXO-292, and BLU-667, where HM06/TAS0953 is administered at a twice-daily dose of 50 mg/kg, and LOXO-292 and BLU-667 are each administered at a twice-daily dose of 30 mg/kg on tumor volume. Data were presented as mean±SE (each group, n=5). The mean tumor volume on day 15 was significantly lower in the agent-treated groups than in the control group (p<0.05, Dunnet test). In addition, the mean tumor volume on day 15 was significantly lower in the HM06/TAS0953 group than in the LOXO-292 and BLU-667 groups (each p<0.05, Turkey test). While LOXO-292 and BLU-667 showed a trend to tumor regrowth at day 15, HM06/TAS0953 exhibited consistent tumor regression. FIG. 14B shows the effects of the dosages on body weight during the treatment period. There were no significant changes in terms of body weight change among groups. Data were presented as mean±SE (each group, n=5).

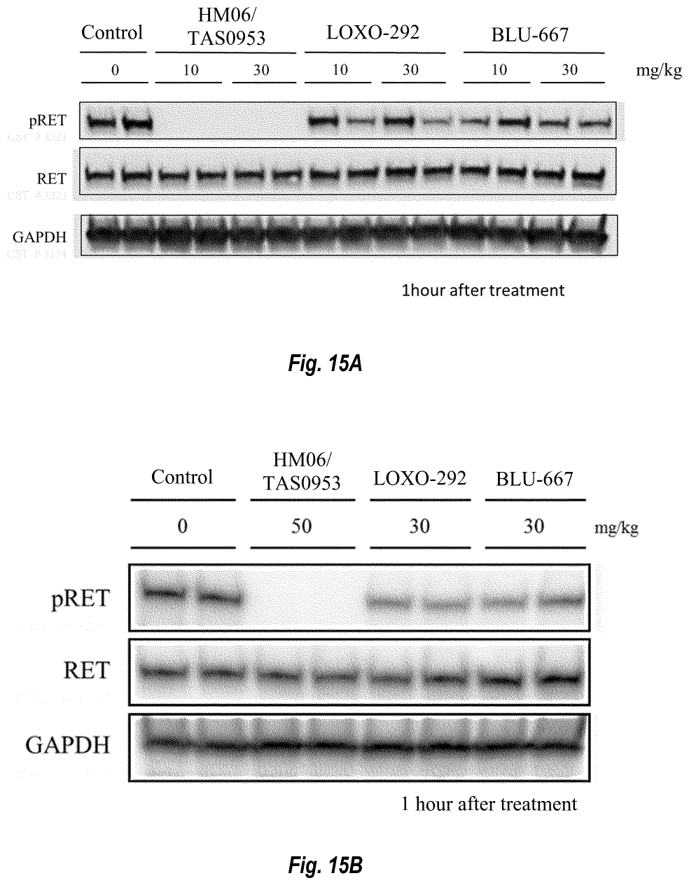

Inhibitory effect of HM06/TAS0953, LOXO-292, and BLU-667 on RET phosphorylation in Ba/F3 KIF5B-RET^{G810R} tumors one hour after administration was evaluated by Western blot. Mice bearing Ba/F3 KIF5B-RET^{G810R} were orally administered HM06 at 10, 30, or 50 mg/kg or LOX0292 and BLU667 at 10 or 30 mg/kg, respectively once. The tumors were collected and lysed at 1 hour post dosing. The cell lysates were immunoblotted to detect phosphorylated RET (pRET), RET, and GAPDH (control). As shown in the Western blots of FIGS. 15A (first study) and 15B (second study), compared with the control group, a marked decrease in RET phosphorylation was observed in the HM06/TAS0953 group whereas a slight decrease was observed in the LOXO-292 and BLU-667 groups.

Western blotting was performed in each of the two studies described above. Tumors were collected 1 hour after administration. Tumor tissues were lysed with Sample Diluent Concentrate 2 (R&D Systems) supplemented with cOmplete™, mini, Protease Inhibitor Cocktail (Roche Applied Science) and PhosSTOP™ Phosphatase Inhibitor Cocktail (Roche Applied Science). The lysate was subjected to SDS-PAGE and transferred to a PVDF membrane (Trans-Blot turbo Blotting System; Bio-rad). Then the membrane was blocked with Blocking One-P (Nacalai Tesque Inc.), and incubated overnight at 4° C. with the primary antibodies. Phospho-Ret (Tyr905) antibody (#3221, Cell Signaling Technology), Ret (C31B4) Rabbit mAb (#3223, Cell Signaling Technology) and GAPDH (D16H11) XP® Rabbit mAb (#5174, Cell Signaling Technology) were used as the primary antibodies. Subsequently, the membrane was washed, incubated with the secondary antibody (#7074, Cell Signaling Technology) for 1 hour at room temperature, and washed again. The chemiluminescence images were obtained by the luminescent image analyzer (Amersham™ Imager 600 QC, GE Healthcare Japan Corporation). GAPDH was used as an internal control.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

1.-82. (canceled)

83. A method of treating a human patient with non-small cell lung cancer (NSCLC) having a RET gene abnormality comprising administering to the human patient a composition comprising 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide, wherein the human patient is administered a dosage equivalent to about 40 mg to about 3000 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day.

84. A method of treating a human patient with locally advanced or metastatic non-small cell lung cancer (NSCLC) having a RET gene abnormality comprising administering to the human patient a composition comprising 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide, wherein the human patient is administered a dosage equivalent to about 40 mg to about 1000 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day.

85. A method of treating a human patient with metastatic non-small cell lung cancer (NSCLC) having a RET gene abnormality with brain and/or leptomeningeal metastases comprising administering to the human patient a composition comprising 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide, wherein the human patient is administered an effective amount of 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide.

86. The method of claim 85, wherein the effective amount is a dosage equivalent to about 40 mg to about 3000 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day.

87. The method of claim 85 or claim 86, wherein the brain and/or leptomeningeal metastases is asymptomatic.

88. A method of treating a human patient with a solid tumor having a RET gene abnormality comprising administering to the human patient a composition comprising 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide, wherein the human patient is administered a dosage equivalent to about 40 mg to about 3000 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day.

89. A method of treating a human patient with a solid tumor having a RET gene abnormality comprising administering to the human patient a composition comprising 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide, wherein the RET gene abnormality comprises a solvent front mutation of a RET protein.

90. A method of treating a human patient with a solid tumor having a RET gene abnormality with brain and/or leptomeningeal metastases comprising administering to the human patient a composition comprising 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide, wherein the human patient is administered an effective amount of 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide.

91. The method of claim 89 or claim 90, wherein the effective amount is a dosage equivalent to about 40 mg to about 3000 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day.

92. The method of claim 90 or claim 91, wherein the brain and/or leptomeningeal metastases is asymptomatic.

93. The method of any one of the preceding claims, wherein the human patient is administered a dosage of 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base per day equivalent to one value selected from: about 150 mg to about 640 mg; 160 mg to about 640 mg; 320 mg to about 640 mg; 480 mg to about 640 mg; about 640 mg; about 480 mg to about 3000 mg; about 480 mg to about 2000 mg; about 480 mg to about 1500 mg; about 480 mg to about 1280 mg; about 480 mg to about 1000 mg; about 640 mg to about 3000 mg; about 640 mg to about 2000 mg; about 640 mg to about 1500 mg; about 640 mg to about 1280 mg; about 640 mg to about 1000 mg; 3000 mg; 2000 mg; 1500 mg; 1280 mg; 1000 mg; about 150 mg to about 500 mg; 160 mg to about 500 mg; about 150 mg; or about 160 mg.

94. The method of any one of the preceding claims, wherein the composition is administered orally.

95. The method of any one of the preceding claims, wherein the composition is administered orally as a single tablet or multiple tablets.

96. The method of claim 95 wherein each tablet comprises a dose equivalent to about 10 or about 50 mg 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base.

97. The method of any one of the preceding claims, wherein the composition comprises the di-hydrochloride salt of 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide.

98. The method of any one of the preceding claims wherein the composition further comprises citric acid, microcrystalline cellulose, lactose, polyvinyl N-pyrrolidone, sodium lauryl sulfate, and/or glyceryl behenate.

99. The method of any one of the preceding claims, wherein the composition is administered once per day (QD) or twice per day (BID).

100. The method of any one of the preceding claims, wherein the composition is administered twice per day (BID).

101. The method of any one of claims 83 to 92 and 94 to 100, wherein the human patient is administered a dosage of 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide free base twice per day (BID), equivalent to one value selected from: about 160 to about 1500 mg; about 160 to about 1000 mg; about 160 to about 750 mg; about 160 to about 640 mg; about 160 to about 500 mg; about 160 to about 320 mg; about 320 mg; about 500 mg; about 640 mg; about 750 mg; about 1000 mg; or about 1500 mg.

102. The method of any one of the preceding claims, wherein the dosage is the same for a patient weighing greater than 50 kg and for a patient weighing less than 50 kg.

103. The method of any one of the preceding claims, wherein the composition is administered in at least one 21-day treatment cycle.

104. The method of any one of the preceding claims, wherein the RET gene abnormality comprises at least one of a RET gene fusion, a point mutation, a deletion mutation, an increased copy number of a RET gene, overexpression of any one or more thereof, and overexpression of a RET gene.

105. The method of any one of the preceding claims, wherein the RET gene abnormality comprises a RET gene fusion.

106. The method of any one of the preceding claims, wherein the RET gene abnormality: comprises a RET gene fusion with a gene encoding CCDC6, KIF5B, or TRIM33; or comprises a resistance mutation of a RET protein; or comprises a solvent front mutation of a RET protein and/or a mutation in the hinge region of a RET protein; or comprises a mutation of a RET protein at amino acid residue 730, 736, 760, 772, 804, 806, 807, 808, 809, 810, and/or 883; or comprises a mutation of a RET protein at amino acid residue 804, 806, 807, 808, 809, and/or 810; or comprises a mutation of a RET protein at amino acid residue 810.

107. The method of any one of the preceding claims, wherein the RET gene abnormality comprises a mutation of a RET protein comprising:
a) a V804X mutation, wherein X is any amino acid other than valine or glutamic acid;
b) a Y806X mutation, wherein X is any amino acid other than tyrosine;
c) a A807X mutation, wherein X is any amino acid other than alanine;
d) a K808X mutation, wherein X is any amino acid other than lysine;
e) a Y809X mutation, wherein X is any amino acid other than tyrosine; and/or
f) a G810X mutation, wherein X is any amino acid other than glycine.

108. The method of any one of the preceding claims, wherein the RET gene abnormality comprises a mutation of a RET protein comprising:
a) a L730Q or L730R mutation;
b) a G736A mutation;
c) a L760Q mutation;
d) a L772M mutation;
e) a V804L or V804M mutation;
f) a Y806C, Y806S, Y806H, or Y806N mutation;
g) a G810R, G810S, G810C, G810V, G810D, or G810A mutation; and/or
h) a A883V mutation.

109. The method of any one of the preceding claims, wherein the RET gene abnormality comprises a mutation of a RET protein comprising:
a) a V804L or V804M mutation;
b) a Y806C, Y806S, Y806H, or Y806N mutation; and/or
c) a G810R, G810S, G810C, G810V, G810D, or G810A mutation.

110. The method of any one of the preceding claims, wherein the RET gene abnormality comprises a G810R, G810S, G810C, G810V, G810D, or G810A mutation of a RET protein.

111. The method of any one of the preceding claims, wherein the RET gene abnormality comprises a G810R mutation of a RET protein.

112. The method of any one of the preceding claims, wherein the cancer or the tumor is resistant to at least one multi-kinase inhibitor, or to at least one RET selective inhibitor, or selpercatinib and/or pralsetinib.

113. The method of any one of the preceding claims, wherein the cancer or the tumor comprises cells resistant to selpercatinib and/or pralsetinib.

114. The method of any one of the preceding claims, wherein the cancer or the tumor is not resistant to 4-amino-N-[4-(methoxymethyl)phenyl]-7-(1-methylcyclopropyl)-6-(3-morpholinoprop-1-yn-1-yl)-7H-pyrrolo[2,3-d]pyrimidine-5-carboxamide and is resistant to at least one other RET selective inhibitor.

115. The method of any one of the preceding claims, wherein the human patient previously received prior treatment for the cancer or the tumor.

116. The method of any one of the preceding claims, wherein the cancer or tumor being treated progressed following a prior treatment for the cancer or tumor.

117. The method of any one of the preceding claims, wherein the human patient is in one or more of the following conditions: has developed intolerance to a prior treatment for the cancer or tumor; previously received a multi-kinase inhibitor; has previously received cabozantinib, vandetanib, lenvatinib, and/or RXDX-105; has previously received a RET selective inhibitor; previously received selpercatinib and/or pralsetinib; has not previously received a RET selective inhibitor; has at least one of salivary gland cancer, lung cancer, colorectal cancer, thyroid cancer, breast cancer, pancreatic cancer, ovarian cancer, skin cancer, and brain cancer; has at least one of medullary or anaplastic thyroid cancer, metastatic breast cancer, and metastatic pancreatic adenocarcinoma.
